(19) United States
(12) Patent Application Publication
(10) Pub. No.: US 2015/0031561 A1
(43) Pub. Date: Jan. 29, 2015

(54) BIOMARKER PANELS, DIAGNOSTIC METHODS AND TEST KITS FOR OVARIAN CANCER

(71) Applicant: Vermillion, Inc., Austin, TX (US)

(72) Inventors: Greg P. Bertenshaw, Gaithesburg, MA (US); Ping F. Yip, Winchester, MA (US); Partha Seshaiah, Kirkland, WA (US)

(73) Assignee: Vermillion, Inc., Austin, TX (US)

(21) Appl. No.: 14/099,522

(22) Filed: Dec. 6, 2013

Related U.S. Application Data
(63) Continuation of application No. 14/000,597, now abandoned.
(60) Provisional application No. 61/463,870, filed on Feb. 24, 2011.

(51) Int. Cl. G01N 33/574; H01J 49/00

(52) U.S. Cl. CPC G01N 33/57449; G01N 33/57488; H01J 49/0027; G01N 2333/8107; G01N 2333/705; G06F 19/3431
USPC 506/9; 506/18; 702/19

(57) ABSTRACT

Methods are provided for predicting the presence, subtype and stage of ovarian cancer, as well as for assessing the therapeutic efficacy of a cancer treatment and determining whether a subject potentially is developing cancer. Associated test kits, computer and analytical systems as well as software and diagnostic models are also provided.

### RELATED APPLICATIONS

This application is a Continuation of U.S. application Ser. No. 14/000,597 filed Aug. 20, 2013, which is the U.S. national phase of PCT International Application No. PCT/US2012/024997, filed Feb. 14, 2012, which published as International Publication No. WO 2012/115820 A2, which claims the benefit of and priority to U.S. Provisional Application No. 61/463,870, filed Feb. 24, 2011, the entire contents of which are incorporated herein by reference.

### STATEMENT OF GOVERNMENT SUPPORT

The subject matter of the present application includes work supported by SBIR Award No. HHSN261200800045C entitled “Improvement of a Promising MAP for Ovarian Cancer” from the National Cancer Institute.

### FIELD OF THE INVENTION

This invention provides methods for predicting and diagnosing ovarian cancer, particularly epithelial ovarian cancer, and it further provides associated analytical reagents, diagnostic models, test kits and clinical reports.

### BACKGROUND

The American Cancer Society estimates that ovarian cancer will strike 22,430 women and take the lives of 15,280 women in 2007 in the United States. Ovarian cancer is not a single disease, however, and there are actually more than 30 types and subtypes of ovarian malignancies, each with its own pathology and clinical behavior. Most experts therefore group ovarian cancers within three major categories, according to the kind of cells from which they were formed: epithelial tumors arise from cells that line or cover the ovaries; germ cell tumors originate from cells that are destined to form eggs within the ovaries; and sex cord-stromal cell tumors begin in the connective cells that hold the ovaries together and produce female hormones.

Common epithelial tumors begin in the surface epithelium of the ovaries and account for about 90 percent of all ovarian cancers in the U.S. (and the following percentages reflect U.S. prevalence of these cancers). They are further divided into a number of subtypes—including serous, endometrioid, mucinous, and clear cell tumors—that can be further subclassified as benign or malignant tumors. Serous tumors are the most widespread forms of ovarian cancer. They account for 40 percent of common epithelial tumors. About 50 percent of these serous tumors are malignant, 33 percent are benign, and 17 percent are of borderline malignancy. Serous tumors occur most often in women who are between 40 and 60 years of age.

Endometrioid tumors represent approximately 20 percent of common epithelial tumors. In about 20 percent of individuals, these cancers are associated with endometrial carcinoma (cancer of the womb lining) In 5 percent of cases, they also are linked with endometriosis, an abnormal occurrence of endometrium (womb lining tissue) within the pelvic cavity. The majority (about 80 percent) of these tumors are malignant, and the remainder (roughly 20 percent) usually is borderline malignancies. Endometrioid tumors occur primarily in women who are between 50 and 70 years of age.

Clear cell tumors account for about 6 percent of common epithelial tumors. Nearly all of these tumors are malignant. Approximately one-half of all clear cell tumors are associated with endometriosis. Most patients with clear cell tumors are between 40 and 80 years of age.

Mucinous tumors make up about 1 percent of all common epithelial tumors. Most (approximately 80 percent) of these tumors are benign, 15 percent are of borderline malignancy, and only 5 percent are malignant. Mucinous tumors appear most often in women between 30 to 50 years of age.

Ovarian cancer is by far the most deadly of gynecologic cancers, accounting for more than 55 percent of all gynecologic cancer deaths. But ovarian cancer is also among the most treatable—if it is caught early. When ovarian cancer is caught early and appropriately treated, the 5-year survival rate is 93 percent. See, for example, Luce et al, “Early Diagnosis Key to Epithelial Ovarian Cancer Detection,” The Nurse Practitioner, December 2003 at p. 41. Extensive background information about ovarian cancer is readily available on the internet, for example, from the “Overview: Ovarian Cancer” of the Cancer Reference Information provided by the American Cancer Society and the NCCN Clinical Practice Guidelines in Oncology™ Ovarian Cancer V.1.2007.

The current reality for the diagnosis of ovarian cancer is that most cases—81 percent of all cases of ovarian cancer—are not caught in earliest stage. This is because early stage ovarian cancer is very difficult to diagnose. Its symptoms may not appear or be noticed at this point. Or, symptoms—such as bloating, indigestion, diarrhea, constipation and others—may be vague and associated with many common and less serious conditions. Most importantly, there has been no effective test for early detection. An effective tool for early and accurate detection of ovarian cancer is a critical unmet medical need.

What has been urgently needed in the field of gynecologic oncology is a minimally invasive (preferably serum-based) clinical test for assessing and predicting the presence of ovarian cancer that is based on a robust set of biomarkers and sample features identified from a large and diverse set of samples, together with methods and associated computer systems and software tools to predict, diagnose and monitor ovarian cancer with high accuracy at its various stages.

### SUMMARY OF THE INVENTION

The present invention generally relates to cancer biomarkers and particularly to biomarkers associated with ovarian cancer. It provides methods to predict, evaluate diagnose, and monitor cancer, particularly ovarian cancer, by measuring certain biomarkers, and further provides a set or array of reagents to evaluate the expression levels of biomarkers that are associated with ovarian cancer. A preferred set of biomarkers provides a detectable molecular signature of ovarian cancer in a subject. The invention provides a predictive or diagnostic test for ovarian cancer, particularly for epithelial ovarian cancer and more particularly for early-stage ovarian cancer (that is Stage I, Stage II or Stage I and II together).

In one aspect, the present disclosure generally features a method of predicting the ovarian cancer status of a subject, involving the steps of measuring the level of CA-125 and HE4 and measuring the level of one or more biomarkers selected from the group consisting of IL-2 receptor alpha (IL-2Rα), Alpha-1-Antitrypsin (AAT), C-Reactive Protein (CRP), YKL-40, Cellular Fibronectin (cFib), prostasin, Tissue Inhibitor of Metalloproteinases 1 (TIMP-1), IL-8, IL-6, Vascular Endothelial Growth Factor B (VEGF-B), Matrix Metalloproteinase-7 (MMP-7), calprotectin, Insulin-like Growth Factor-Binding Protein 2 (IGFBP-2), Lectin-Like Oxidized LDL Receptor 1 (LOX-1), neuropilin-1, TNFR2, and MPIF-1 in a sample of a biological fluid obtained from the subject; and correlating the measurements with ovarian cancer status.

In another aspect, the present disclosure features a kit containing a panel of affinity reagents that each selectively binds to CA-125 and HE4 and one or more biomarkers selected from the group consisting of Interleukin-2 receptor alpha (IL-2 receptor alpha), Alpha-1-Antitrypsin (AAT), C-Reactive Protein (CRP), YKL-40, Cellular Fibronectin (cFib), Cancer Antigen 72-4 (CA-72-4), prostasin, Tissue Inhibitor of Metalloproteinases 1 (TIMP-1), IL-8, Matrix Metalloproteinase-7 (MMP-7), IL-6, Vascular Endothelial Growth Factor B (VEGF-B), calprotectin, Insulin-like Growth Factor-Binding Protein 2 (IGFBP-2), Lectin-Like Oxidized LDL Receptor 1 (LOX-1), neuropilin-1, TNFR2, and MPIF-1; and a panel of containers each comprising CA-125 and HE4 and a one or more biomarkers selected from the group consisting of Interleukin-2 receptor alpha (IL-2 receptor alpha), Alpha-1-Antitrypsin (AAT), C-Reactive Protein (CRP), YKL-40, Cellular Fibronectin (cFib), Cancer Antigen 72-4 (CA-72-4), prostasin, Tissue Inhibitor of Metalloproteinases 1 (TIMP-1), IL-8, Matrix Metalloproteinase-7 (MMP-7), IL-6, Vascular Endothelial Growth Factor B (VEGF-B), calprotectin, Insulin-like Growth Factor-Binding Protein 2 (IGFBP-2), Lectin-Like Oxidized LDL Receptor 1 (LOX-1), neuropilin-1, TNFR2, and MPIF-1.

In a further aspect, the present disclosure features a panel of purified peptides containing CA-125 and HE4 and one or more biomarkers selected from the group consisting of Interleukin-2 receptor alpha (IL-2 receptor alpha), Alpha-1-Antitrypsin (AAT), C-Reactive Protein (CRP), YKL-40, Cellular Fibronectin (cFib), Cancer Antigen 72-4 (CA-72-4), prostasin, Tissue Inhibitor of Metalloproteinases 1 (TIMP-1), IL-8, Matrix Metalloproteinase-7 (MMP-7), IL-6, Vascular Endothelial Growth Factor B (VEGF-B), calprotectin, Insulin-like Growth Factor-Binding Protein 2 (IGFBP-2), Lectin-Like Oxidized LDL Receptor 1 (LOX-1), neuropilin-1, TNFR2, and MPIF-1.

In various embodiments of any of the above aspects or any other aspect of the disclosure delineated herein, the methods involve measuring the level of Cancer Antigen 72-4 (CA-72-4). In another embodiment the ovarian cancer status is presence of ovarian cancer. In additional embodiments the ovarian cancer is stage I ovarian cancer. In yet another embodiment the ovarian cancer is stage II ovarian cancer. In other embodiments the ovarian cancer is stage III ovarian cancer. In yet another embodiment the ovarian cancer is stage IV ovarian cancer. In further embodiments the ovarian cancer is stage I, II, III, or IV ovarian cancer. In other embodiments the method further involves managing subject treatment based on the status. In yet another embodiment managing subject treatment is selected from the group consisting of ordering more tests, performing surgery, and taking no further action. In yet another embodiment the method includes measuring the level of CA-125 and HE4 and measuring the level of one or more biomarkers selected from the group consisting of Interleukin-2 receptor alpha (IL-2 receptor alpha), Alpha-1-Antitrypsin (AAT), C-Reactive Protein (CRP), YKL-40, Cellular Fibronectin (cFib), Cancer Antigen 72-4 (CA-72-4), prostasin, Tissue Inhibitor of Metalloproteinases 1 (TIMP-1), IL-8, Matrix Metalloproteinase-7 (MMP-7), IL-6, Vascular Endothelial Growth Factor B (VEGF-B), calprotectin, Insulin-like Growth Factor-Binding Protein 2 (IGFBP-2), Lectin-Like Oxidized LDL Receptor 1 (LOX-1), neuropilin-1, TNFR2, and MPIF-1 in a sample of a biological fluid obtained from the subject after subject management; correlating the measurements with ovarian cancer status; and determining if subject management resulted in a change in ovarian cancer status. In other embodiments measuring is selected from detecting the presence or absence of the biomarkers, quantifying the amount of biomarkers, and qualifying the type of biomarker. In certain embodiments the biomarkers are measured by an immunoassay. In further embodiments the correlating is performed by a software classification algorithm. In yet another embodiment the sample is selected from blood, serum, and plasma. In some embodiments the affinity reagent is an antibody. In yet another embodiment the kits further include written instructions for using the affinity reagent to measure the levels of the biomarkers in a sample from a subject. In yet another embodiment the kits include written instructions for use of the kit for determining a subjects ovarian cancer status. In certain embodiments one or more of the peptides have a detectable label.

In a preferred embodiment of the present invention, a method of predicting the ovarian cancer status of a subject is provided, which comprises the steps of: determining the concentration of CA-125 and HE4 in a sample of a biological fluid from the subject and the age of the subject (collectively, the “biomarkers”); and evaluating the biomarkers, wherein a change in the level or evaluation of the biomarkers, as compared with a control group of patients who do not have ovarian cancer, predicts that the subject has ovarian cancer. In a more preferred embodiment, the foregoing method further comprises the evaluation of a subject's menopausal status of the subject as being either post-menopausal or not post-menopausal, and the concentrations of CA15-3 and CA72-4 in a sample of a biological fluid from the subject.

A variety of additional biomarkers also are evaluated with the foregoing biomarkers in additional embodiments of the present invention. These include: Vascular Endothelial Growth Factor (VEGF), Interleukin-2 receptor alpha (IL-2 receptor alpha), Insulin-like Growth Factor-Binding Protein 2 (IGFBP-2), Haptoglobin, Ferritin (FRTN), Prostasin, Interleukin-8 (IL-8), Maspin, Osteopontin, Serum Amyloid P-Component (SAP), Platelet-Derived Growth Factor BB (PDGF-BB) and B cell-activating factor (BAFF). Optionally, certain additional biomarkers are also evaluated: Calprotectin, von Willebrand Factor (vWF), Alpha-1-Antitrypsin (AAT), C-Reactive Protein (CRP), Interleukin-6 (IL-6), Leptin, Transthyretin (TTR), Carcinoembryonic Antigen (CEA), Insulin-like Growth Factor-Binding Protein 1 (IGFBP-1) and Thyroxine-Binding Globulin (TBG).

In preferred embodiments, the evaluation is made by a method selected from the group consisting of: logistic regression, look-up tables, decision tree, support vector machine, cluster analysis, neighbor analysis, genetic algorithm, Bayesian and non-Bayesian approaches, and the like. Additionally, the sample preferably is selected from the group of fluids and tissues drawn from a patient that include blood, serum, plasma, lymph, cerebrospinal fluid, ascites, urine and tissue biopsy.

In other preferred embodiments, the methods of the present invention also include the step of providing a written or electronic report of the prediction of ovarian cancer and, optionally, the report includes a prediction as to the presence or absence or likelihood of ovarian cancer in the subject or the stratified risk of ovarian cancer for the subject, optionally by stage of cancer.

Other preferred embodiments provide a method in which the a) the sum of sensitivity and specificity for the method is greater than about 150%, when the sensitivity is above about 95%; or b) the sum of sensitivity and specificity for the method is greater than about 170%, when the specificity is above 95%; and c) the foregoing sum of sensitivity and specificity is supported by analysis of a set of samples comprising at least about 50 cancer samples and 150 benign samples.

Sets of reagents, test kits and multianalyte and ELISA panels and kits are provided to accomplish the foregoing methods.

Other biomarkers useful in the methods of the present invention include the following, which may be determined as one or more additional markers in the methods of claims 1 through 4 appended below: Prostatic Acid Phosphatase (PAP), Epidermal Growth Factor Receptor (EGFR), Cathepsin D, YKL-40, Matrix Metalloproteinase-7 (MMP-7), Vascular Endothelial Growth Factor D (VEGF-D), Tissue Inhibitor of Metalloproteinases 1 (TIMP-1), Mesothelin (MSLN), Sortilin, Cellular Fibronectin (cFib), Osteoprotegerin (OPG), EN-RAGE, CD 40 antigen (CD40), Lectin-Like Oxidized LDL Receptor 1 (LOX-1), Neuropilin-1, Fetuin-A, Resistin, Matrix Metalloproteinase-2 (MMP-2), Peroxiredoxin 4 (Prx-IV), Phosphoserine Aminotransferase (PSAT), Alpha-1-Microglobulin (AlMicro), Heparin-Binding EGF-Like Growth Factor (HB-EGF), Hepatocyte Growth Factor (HGF), Trefoil Factor 3 (TFF3), Complement Factor 11, Clusterin (CLU), Aldose Reductase, Macrophage Migration Inhibitory Factor (MW), Amphiregulin (AR), Macrophage Inflammatory Protein-1 alpha (MIP-1 alpha), FASLG Receptor (FAS), Vascular Endothelial Growth Factor Receptor 1 (VEGFR-1), Matrix Metalloproteinase-1 (MMP-1), Monocyte Chemotactic Protein 2 (MCP-2) and Vascular Endothelial Growth Factor B (VEGF-B).

In yet other embodiments of the present invention, any three or more of the following biomarkers are determined and evaluated: HE4, CA-125, IL-2 receptor alpha, AAT, CRP, YKL-40, fibronectin and CA-72-4.

In another embodiment of the invention, the levels of the following biomarkers, optionally including HE4, are determined and evaluated, in some cases with a determination of age: CA-125, CA72-4, VEGF-B, Maspin, VEGF-D and YKL-40; CA-125, CA72-4, VEGF-B, Maspin, VEGF-D, YKL-40, OSP, Age and CRP; CA-125, CA72-4, VEGF-B, Maspin, and OSP; CA-125, CA72-4, VEGF-B, Maspin, YKL-40 and Age; CA-125, Maspin and Age; CA-125, CA72-4, VEGF-B, Maspin, OSP and Age; CA-125, VEGF-B and Age.

In an additional embodiment of the invention, the levels of the following biomarkers, optionally including HE4, are determined and evaluated: HE4, Cancer Antigen 125 (CA-125), Cancer Antigen 72-4 (CA-72-4), Cancer Antigen 15-3 (CA-15-3), Age, Insulin-like Growth Factor-Binding Protein 2 (IGFBP-2), Interleukin-2 receptor alpha (IL-2 receptor alpha); HE4, Cancer Antigen 125 (CA-125) and YKL-40, optionally also including Age; HE4, Cancer Antigen 72-4(CA-72-4), Cancer Antigen 15-3 (CA-15-3), Age, Insulin-like Growth Factor-Binding Protein 2 (IGFBP-2), and Interleukin-2 receptor alpha (IL-2 receptor alpha); CA-125, CA-72-4, Prostasin, CA-15-3, Age, IL-2 receptor alpha, IL-8, optionally also including HE4; and CA-125, CA-72-4, Prostasin, CA-15-3, Age, IL-2 receptor alpha, IL-8, FRTN. VEGF, Osteopontin, Maspin, and Haptoglobin, optionally also including Age.

More specifically, predictive tests and associated methods and products also provide useful clinical information regarding the stage of ovarian cancer progression, that is: Stage I, Stage II, Stage III and Stage IV and an advanced stage which reflects relatively advanced tumors that cannot readily be classified as either Stage III or Stage IV. Overall, the invention also relates to newly discovered correlations between the relative levels of expression of certain groups of markers in bodily fluids, preferably blood serum and plasma, and a subject's ovarian cancer status.

In one embodiment, the invention provides a set of reagents to measure the expression levels of a panel or set of biomarkers in a fluid sample drawn from a patient, such as blood, serum, plasma, lymph, cerebrospinal fluid, ascites or urine. The reagents in a further embodiment are a multianalyte panel assay comprising reagents to evaluate the expression levels of these biomarker panels.

In embodiments of the invention, a subject's sample is prepared from tissue samples such a tissue biopsy or from primary cell cultures or culture fluid. In a further embodiment, the expression of the biomarkers is determined at the polypeptide level. Related embodiments utilize immunoassays, enzyme-linked immunosorbent assays and multiplexed immunoassays for this purpose.

Preferred panels of biomarkers are selected from the group consisting of the following sets of molecules and their measurable fragments: (a) myoglobin, CRP (C reactive protein), FGF basic protein and CA 19-9; (b) Hepatitis C NS4, Ribosomal P Antibody and CRP; (c) CA 19-9, TGF alpha, EN-RAGE, EGF and HSP 90 alpha antibody, (d) EN-RAGE, EGF, CA 125, Fibrinogen, Apolipoprotein CIII, EGF, Cholera Toxin and CA 19-9; (e) Proteinase 3 (cANCA) antibody, Fibrinogen, CA 125, EGF, CD40, TSH, Leptin, CA 19-9 and lymphotactin; (f) CA125, EGFR, CRP, IL-18, Apolipoprotein CIII, Tenascin C and Apolipoprotein A1; (g) CA125, Beta-2 Microglobulin, CRP, Ferritin, TIMP-1, Creatine Kinase-MB and IL-8; (h) CA125, EGFR, IL-10, Haptoglobin, CRP, Insulin, TIMP-1, Ferritin, Alpha-2 Macroglobulin, Leptin, IL-8, CTGF, EN-RAGE, Lymphotactin, TNF-alpha, IGF-1, TNF RII, von Willebrand Factor and MDC; (i) CA-125, CRP, EGF-R, CA-19-9, Apo-AI, Apo-CIII, IL-6, IL-18, MIP-1a, Tenascin C and Myoglobin; (j) CA-125, CRP, EGF-R, CA-19-9, Apo-AI, Apo-CIII, IL-6, MIP-1a, Tenascin C and Myoglobin; and (k) any of the biomarker panels presented in Table II and Table III.

In another embodiment, the reagents that measure such biomarkers may measure other molecular species that are found upstream or downstream in a biochemical pathway or measure fragments of such biomarkers and molecular species. In some instances, the same reagent may accurately measure a biomarker and its fragments.

Another embodiment of the present invention relates to binding molecules (or binding reagents) to measure the biomarkers and related molecules and fragments. Contemplated binding molecules includes antibodies, both monoclonal and polyclonal, aptamers and the like.

Other embodiments include such binding reagents provided in the form of a test kit, optionally together with written instructions for performing an evaluation of biomarkers to predict the likelihood of ovarian cancer in a subject.

In other of its embodiments, the present invention provides methods of predicting the likelihood of ovarian cancer in a subject based on detecting or measuring the levels in a specimen or biological sample from the subject of the foregoing biomarkers. As described in this specification, a change in the expression levels of these biomarkers, particularly their relative expression levels, as compared with a control group of patients who do not have ovarian cancer, is predictive of ovarian cancer in that subject.

In other of its aspects, the type of ovarian cancer that is predicted is serous, endometrioid, mucinous, and clear cell tumors. And prediction of ovarian cancer includes the prediction of a specific stage of the disease such as Stage I (IA, IB or IC), II, III and IV tumors.

In yet another embodiment, the invention relates to creating a report for a physician of the relative levels of the biomarkers and to transmitting such a report by mail, fax, email or otherwise. In an embodiment, a data stream is transmitted via the internet that contains the reports of the biomarker evaluations. In a further embodiment, the report includes the prediction as to the presence or absence of ovarian cancer in the subject or the stratified risk of ovarian cancer for the subject, optionally by subtype or stage of cancer.

According to another aspect of the invention, the foregoing evaluation of biomarker expression levels is combined for diagnostic purposes with other diagnostic procedures such as gastrointestinal tract evaluation, chest x-ray, HE4 test, CA-125 test, complete blood count, ultrasound or abdominal/pelvic computerized tomography, blood chemistry profile and liver function tests.

Yet other embodiments of the invention relate to the evaluation of samples drawn from a subject who is symptomatic for ovarian cancer or is at high risk for ovarian cancer. Other embodiments relate to subjects who are asymptomatic of ovarian cancer. Symptomatic subjects have one or more of the following: pelvic mass; ascites; abdominal distention; general abdominal discomfort and/or pain (gas, indigestion, pressure, swelling, bloating, cramps); nausea, diarrhea, constipation, or frequent urination; loss of appetite; feeling of fullness even after a light meal; weight gain or loss with no known reason; and abnormal bleeding from the vagina. The levels of biomarkers may be combined with the findings of such symptoms for a diagnosis of ovarian cancer.

Embodiments of the invention are highly accurate for determining the presence of ovarian cancer. By “highly accurate” is meant a sensitivity and a specificity each at least about 85 percent or higher, more preferably at least about 90 percent or 92 percent and most preferably at least about 95 percent or 97 percent accurate Embodiments of the invention further include methods having a sensitivity of at least about 85 percent, 90 percent or 95 percent and a specificity of at least about 55 percent, 65 percent, 75 percent, 85 percent or 90 percent or higher. Other embodiments include methods having a specificity of at least about 85 percent, 90 percent or 95 percent, and a sensitivity of at least about 55 percent, 65 percent, 75 percent, 85 percent or 90 percent or higher.

Embodiments of the invention relating sensitivity and specificity are determined for a population of subjects who are symptomatic for ovarian cancer and have ovarian cancer as compared with a control group of subjects who are symptomatic for ovarian cancer but who do not have ovarian cancer. In another embodiment, sensitivity and specificity are determined for a population of subjects who are at increased risk for ovarian cancer and have ovarian cancer as compared with a control group of subjects who are at increased risk for ovarian cancer but who do not have ovarian cancer. And in another embodiment, sensitivity and specificity are determined for a population of subjects who are symptomatic for ovarian cancer and have ovarian cancer as compared with a control group of subjects who are not symptomatic for ovarian cancer but who do not have ovarian cancer.

In other aspects, the levels of the biomarkers are evaluated by applying a statistical method such as knowledge discovery engine (KDE™), regression analysis, discriminant analysis, classification tree analysis, random forests, ProteomeQuest®, support vector machine, One R, kNN and heuristic naive Bayes analysis, neural nets and variants thereof.

In another embodiment, a predictive or diagnostic model based on the expression levels of the biomarkers is provided. The model may be in the form of software code, computer readable format or in the form of written instructions for evaluating the relative expression of the biomarkers.

A patient's physician can utilize a report of the biomarker evaluation, in a broader diagnostic context, in order to develop a relatively more complete assessment of the risk that a given patient has ovarian cancer. In making this assessment, a physician will consider the clinical presentation of a patient, which includes symptoms such as a suspicious pelvic mass and/or ascites, abdominal distention and other symptoms without another obvious source of malignancy. The general lab workup for symptomatic patients currently includes a GI evaluation if clinically indicated, chest x-ray, CA-125 test, CBC, ultrasound or abdominal/pelvic CT if clinically indicated, chemistry profile with LFTs and may include a family history evaluation along with genetic marker tests such as BRCA-1 and BRCA-2. (See, generally, the NCCN Clinical Practice Guidelines in Oncology™ for Ovarian Cancer, V.I.2007.)

The present invention provides a novel and important additional source of information to assist a physician in stratifying a patient's risk of having ovarian cancer and in planning the next diagnostic steps to take. The present invention is also similarly useful in assessing the risk of ovarian cancer in non-symptomatic, high-risk subjects as well as for the general population as a screening tool. It is contemplated that the methods of the present invention may be used by clinicians as part of an overall assessment of other predictive and diagnostic indicators.

The present invention also provides methods to assess the therapeutic efficacy of existing and candidate chemotherapeutic agents and other types of cancer treatments. As will be appreciated by persons skilled in the art, the relative expression levels of the biomarker panels—or biomarker profiles—are determined as described above, in specimens taken from a subject prior to and again after treatment or, optionally, at progressive stages during treatment. A change in the relative expression of these biomarkers to a non-cancer profile of expression levels (or to a more nearly non-cancer expression profile) or to a stable, non-changing profile of relative biomarker expression levels is interpreted as therapeutic efficacy. Persons skilled in the art will readily understand that a profile of such expressions levels may become diagnostic for cancer or a pre-cancer, pre-malignant condition or simply move toward such a diagnostic profile as the relative ratios of the biomarkers become more like a cancer-related profile than previously.

In another embodiment, the invention provides a method for determining whether a subject potentially is developing cancer. The relative levels of expression of the biomarkers are determined in specimens taken from a subject over time, whereby a change in the biomarker expression profile toward a cancer profile is interpreted as a progression toward developing cancer.

The expression levels of the biomarkers of a specimen may be stored electronically once a subject's analysis is completed and recalled for such comparison purposes at a future time.

The present invention further provides methods, software products, computer systems and networks, and associated instruments that provide a highly accurate test for ovarian cancer.

The combinations of markers described in this specification provide sensitive, specific and accurate methods for predicting the presence of or detecting ovarian cancer at various stages of its progression. The evaluation of samples as described may also correlate with the presence of a pre-malignant or a pre-clinical condition in a patient. Thus, it is contemplated that the disclosed methods are useful for predicting or detecting the presence of ovarian cancer in a sample, the absence of ovarian cancer in a sample drawn from a subject, the stage of an ovarian cancer, the grade of an ovarian cancer, the benign or malignant nature of an ovarian cancer, the metastatic potential of an ovarian cancer, the histological type of neoplasm associated with the ovarian cancer, the indolence or aggressiveness of the cancer, and other characteristics of ovarian cancer that are relevant to prevention, diagnosis, characterization, and therapy of ovarian cancer in a patient.

It is further contemplated that the methods disclosed are also useful for assessing the efficacy of one or more test agents for inhibiting ovarian cancer, assessing the efficacy of a therapy for ovarian cancer, monitoring the progression of ovarian cancer, selecting an agent or therapy for inhibiting ovarian cancer, monitoring the treatment of a patient afflicted with ovarian cancer, monitoring the inhibition of ovarian cancer in a patient, and assessing the carcinogenic potential of a test compound by evaluating biomarkers of test animals following exposure.

### BRIEF DESCRIPTION OF THE DRAWINGS

FIG. 1 is a table showing the demographics of the study subjects.

FIG. 2, comprising FIGS. 2A-2C, is a table showing the biomarkers assayed in the study.

FIG. 3 is a table showing the Area Underneath the Curve (AUC) values from Receiver Operating Characteristic (ROC) curve analysis of the top 20 markers.

FIGS. 4, comprising FIGS. 4A-4E, is a table listing the informative biomarkers identified with Area Underneath the Curve (AUC) values statistically greater than 0.5.

FIG. 5 is a set of graphs showing the Receiver Operating Characteristic curves for the nine most informative biomarkers with area under the curve values greater than 0.800.

FIG. 6, comprising FIGS. 6A and 6B, is a set of graphs showing the serum level distributions broken out by International Federation of Gynecology and Obstetrics (FIGO) ovarian cancer stage for the nine most informative biomarkers with area underneath the curve values greater than 0.800.

FIG. 7, comprising FIGS. 7A and 7B, is a set of graphs showing the serum level distributions broken out by subtype of ovarian cancer stage for the nine most informative biomarkers with area underneath the curve values greater than 0.800.

FIG. 8 is a correlation matrix for biomarkers with area underneath the curve values greater than 0.600.

FIG. 9 is a table listing the identities of markers in clusters A through D.

FIG. 10 is a table showing the correlation data of the markers in cluster A.

FIG. 11 is a table showing the correlation data of the markers in cluster B.

FIG. 12 is a table showing the correlation data of the markers in cluster C.

FIG. 13, comprising FIGS. 13A-13D, is a table showing the correlation data of the markers in cluster D.

FIG. 14 is a table showing the sensitivity at landmark threshold specificity values of logistic regression models using the nine most informative markers and the OVA1 biomarkers.

FIG. 15 is a table showing the specificity at landmark threshold sensitivity values of logistic regression models using the nine most informative markers and the OVA1 biomarkers.

FIG. 16 is a table showing the Area Underneath the Curve (AUC) values from Receiver Operating Characteristic (ROC) curve analysis of the top 20 markers broken out by menopausal status.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Biomarker panel” refers to one of the biomarker panels set forth herein. A preferred biomarker panel comprises CA-125 and HE4 and one or more biomarkers selected from the group consisting of Interleukin-2 receptor alpha (IL-2 receptor alpha), Alpha-1-Antitrypsin (AAT), C-Reactive Protein (CRP), YKL-40, Cellular Fibronectin (cFib), Cancer Antigen 72-4 (CA-72-4), prostasin, Tissue Inhibitor of Metalloproteinases 1 (TIMP-1), IL-8, Matrix Metalloproteinase-7 (MMP-7), IL-6, Vascular Endothelial Growth Factor B (VEGF-B), calprotectin, Insulin-like Growth Factor-Binding Protein 2 (IGFBP-2), Lectin-Like Oxidized LDL Receptor 1 (LOX-1), neuropilin-1, TNFR2, and MPIF-1.

“Eluant” or “wash solution” refers to an agent, typically a solution, which is used to affect or modify adsorption of an analyte to an affinity reagent and/or remove unbound materials from the reagent. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Marker” in the context of the present invention refers to a polypeptide (of a particular apparent molecular weight), which is differentially present in a sample taken from patients having human cancer as compared to a comparable sample taken from control subjects (e.g., a person with a negative diagnosis or undetectable cancer, normal or healthy subject). The term “biomarker” is used interchangeably with the term “marker.”

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to SELDI and immunoassay. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

The phrase “differentially present” refers to differences in the quantity and/or the frequency of a marker present in a sample taken from patients having human cancer as compared to a control subject. Furthermore, a marker can be a polypeptide, which is detected at a higher frequency or at a lower frequency in samples of human cancer patients compared to samples of control subjects. A marker can be differentially present in terms of quantity, frequency or both.

A polypeptide is differentially present between two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between two sets of samples if the frequency of detecting the polypeptide in the ovarian cancer patients' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition, i.e., ovarian cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/m) or a relative amount (e.g., relative intensity of signals).

A “diagnostic amount” of a marker refers to an amount of a marker in a subject's sample that is consistent with a diagnosis of ovarian cancer. A diagnostic amount can be either in absolute amount (e.g., μg/m) or a relative amount (e.g., relative intensity of signals).

A “control amount” of a marker can be any amount or a range of amount, which is to be compared against a test amount of a marker. For example, a control amount of a marker can be the amount of a marker in a person without ovarian cancer. A control amount can be either in absolute amount (e.g., μg/m) or a relative amount (e.g., relative intensity of signals).

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

As used herein by the term a “sample” is meant material which can be specifically related to a patient and from which specific information about the patient can be determined, calculated or inferred. A sample can be composed in whole or in part of biological material from of the patient. A sample can also be material that has contacted the patient in a way that allows tests to be conducted on the sample which provides information about the patient. A sample may also be material that has contacted other material that is not of the patient but allows the first material to then be tested to determine information about the patient. A sample can contact sources of biologic material other than the patient provided that one skilled in the art can nevertheless determine information about the patient from the sample. It is also understood that extraneous material or information that is not the sample could be utilized to conclusively link the patient to the sample. For a non-limiting example, a double blind test requires a chart or database to match a sample with a patient.

As used herein the term “body fluid” it is meant a material obtained from a patient that is substantially fluid in consistency, but may have solid or particulate matter associated with it. A body fluid can also contain material and portions that are not from the patient. For instance a body fluid can be diluted with water, or can contain preservative, such as EDTA. Non-limiting examples of body fluids blood, serum, serosal fluids, plasma, lymph, urine, cerebrospinal fluid, saliva, mucosal secretions of the secretory tissues and organs, vaginal secretions, breast milk, tears, and ascites fluids such as those associated with non-solid tumors. Additional examples include fluids of the pleural, pericardial, peritoneal, abdominal and other body cavities, and the like. Biological fluids may further include liquid solutions contacted with a subject or biological source, for example, cell and organ culture medium including cell or organ conditioned medium, lavage fluids and the like.

“Managing subject treatment” refers to the behavior of the clinician or physician subsequent to the determination of ovarian cancer status. For example, if the result of the methods of the present invention is inconclusive or there is reason that confirmation of status is necessary, the physician may order more tests. Alternatively, if the status indicates that surgery is appropriate, the physician may schedule the patient for surgery. Likewise, if the status is negative, e.g., late stage ovarian cancer or if the status is acute, no further action may be warranted. Furthermore, if the results show that treatment has been successful, no further management may be necessary.

The term “stage” or “cancer stage” is intended to mean a classification of ovarian cancer that is based on the size, invasiveness, progression, migration, etc. of cancer in a subject. The stages of ovarian cancer are well defined. Stage I refers to ovarian cancer wherein the cancer is still contained within the ovary (or ovaries). Specifically, stage IA cancer has developed in one ovary, and the tumor is confined to the inside of the ovary. There is no cancer on the outer surface of the ovary. Laboratory examination of washings from the abdomen and pelvis did not find any cancer cells. Stage IB cancer has developed within both ovaries without any tumor on their outer surfaces. Laboratory examination of washings from the abdomen and pelvis did not find any cancer cells. Stage IC cancer is present in one or both ovaries and 1 or more of the following are present: cancer on the outer surface of at least one of the ovaries; in the case of cystic tumors (fluid-filled tumors), the capsule (outer wall of the tumor) has ruptured (burst); or laboratory examination found cancer cells in fluid or washings from the abdomen.

Stage II cancer is in one or both ovaries and has involved other organs (such as the uterus, fallopian tubes, bladder, the sigmoid colon, or the rectum) within the pelvis. Specifically, stage IIA cancer has spread to or has actually invaded the uterus or the fallopian tubes, or both. Laboratory examination of washings from the abdomen did not find any cancer cells. Stage IIB cancer has spread to other nearby pelvic organs such as the bladder, the sigmoid colon, or the rectum. Laboratory examination of fluid from the abdomen did not find any cancer cells. Stage IIC cancer has spread to pelvic organs as in stages IIA or IIB and laboratory examination of the washings from the abdomen found evidence of cancer cells.

Stage III cancer involves 1 or both ovaries, and 1 or both of the following are present: (1) cancer has spread beyond the pelvis to the lining of the abdomen; (2) cancer has spread to lymph nodes. The cancer is Stage IIIA if, during the staging operation, the surgeon can see cancer involving the ovary or ovaries, but no cancer is grossly visible (can be seen without using a microscope) in the abdomen and the cancer has not spread to lymph nodes. However, when biopsies are checked under a microscope, tiny deposits of cancer are found in the lining of the upper abdomen. Stage BIB cancer is in one or both ovaries, and deposits of cancer large enough for the surgeon to see, but smaller than 2 cm (about ¾ inch) across, are present in the abdomen. Cancer has not spread to the lymph nodes. For a cancer to be stage IIIC the cancer is in one or both ovaries, and one or both of the following are present: cancer has spread to lymph nodes and/or deposits of cancer larger than 2 cm (about ¾ inch) across are seen in the abdomen.

Stage IV cancer is the most advanced stage of ovarian cancer. The cancer is in one or both ovaries. Distant metastasis (spread of the cancer to the inside of the liver, the lungs, or other organs located outside of the peritoneal cavity) has occurred. Finding ovarian cancer cells in pleural fluid (from the cavity that surrounds the lungs) is also evidence of stage IV disease.

As used herein, the term “recurrent ovarian cancer” is intended to mean that the disease has come back (recurred) after completion of treatment.

### Biomarkers

By “CA-125” is meant a polypeptide biomarker having at least 85% sequence identity to NCBI accession numbers NP_{—}078966.2 or AAL65133 or a fragment thereof. An exemplary sequence of CA-125 is:

| | |
|---|---|
| 1 | mlkpsglpgs ssptrslmtg srstkatpem dsgltgatls pktstgaivv |
| | tehtlpftsp |
| 61 | dktlasptss vvgrttqslg vmssalpest srgmthseqr tspslspqvn |
| | gtpsrnypat |
| 121 | smvsglsspr trtsstegnf tkeastytlt vettsgpvte kytvptetst |
| | tegdstetpw |
| 181 | dtryipvkit spmktfadst askenapvsm tpaettvtds htpgrtnpsf |
| | gtlyssfldl |
| 241 | spkgtpnsrg etslelilst tgypfsspep gsaghsrist saplsssasv |
| | ldnkisetsi |
| 301 | fsgqsltspl spgvpearas tmpnsaipfs mtlsnaetsa ervrstissl |
| | gtpsistkqt |
| 361 | aetiltfhaf aetmdipsth iaktlasewl gspgtlggts tsaltttsps |
| | ttlvseetnt |
| 421 | hhstsgkete gtlntsmtpl etsapgeese mtatlvptlg fttldskirs |
| | psqvssshpt |
| 481 | relrttgsts grqssstaah gssdilratt sstskasswt sestaqqfse |
| | pqhtqwvets |
| 541 | psmkterppa stsvaapitt svpsvvsgft tlktsstkgi wleetsadtl |
| | igestagptt |
| 601 | hqfavptgis mtggsstrgs qgtthlltra tassetsadl tlatngvpvs |
| | vspavsktaa |
| 661 | gssppggtkp sytmvssvip etsslqssaf regtslgltp lntrhpfssp |
| | epdsaghtki |
| 721 | stsipllssa svledkvsat stfshhkats sittgtpeis tktkpssavl |
| | ssmtlsnaat |
| 781 | spervrnats plthpspsge etagsvltls tsaettdspn ihptgtltse |
| | ssespstlsl |
| 841 | psvsgvkttf ssstpsthlf tsgeeteets npsvsqpets vsrvrttlas |
| | tsvptpvfpt |
| 901 | mdtwptrsaq fssshlvsel ratsstsvtn stgsalpkis hltgtatmsq |
| | tnrdtfndsa |
| 961 | apqsttwpet sprfktglps atttvstsat slsatvmvsk ftspatssme |
| | atsirepstt |
| 1021 | ilttettngp gsmavastni pigkgyiteg rldtshlpig ttassetsmd |
| | ftmakesvsm |
| 1081 | svspsqsmda agsstpgrts qfvdtfsddv yhltsreiti prdgtssalt |
| | pqmtathpps |
| 1141 | pdpgsarstw lgilssspss ptpkvtmsst fstqrvttsm imdtvetsrw |
| | nmpnlpstts |
| 1201 | ltpsniptsg aigkstlvpl dtpspatsle asegglptls typestntps |
| | ihlgahasse |
| 1261 | spstikltma svvkpgsytp ltfpsiethi hvstarmays sgsspemtap |
| | getntgstwd |
| 1321 | pttyitttdp kdtssaqvst phsvrtlrtt enhpktesat paaysgspki |
| | ssspnltspa |
| 1381 | tkawtitdtt ehstqlhytk laekssgfet qsapgpvsvv iptsptigss |
| | tleltsdvpg |
| 1441 | eplvlapseq ttitlpmatw lstslteema stdldissps spmstfaifp |
| | pmstpshels |
| 1501 | kseadtsair ntdsttldqh lgirslgrtg dlttvpitpl tttwtsvieh |
| | stqaqdtlsa |
| 1561 | tmspthvtqs lkdqtsipas aspshltevy pelgtqgrss seattfwkps |
| | tdtlsreiet |
| 1621 | gptniqstpp mdntttgsss sgvtlgiahl pigtsspaet stnmalerrs |
| | statvsmagt |
| 1681 | mgllvtsapg rsisqslgrv ssvlsestte gvtdsskgss prlntqgnta |
| | lssslepsya |
| 1741 | egsqmstsip ltsspttpdv efiggstfwt kevttvmtsd iskssartes |
| | ssatlmstal |
| 1801 | gstentgkek lrtasmdlps ptpsmevtpw isltlsnapn ttdsldlshg |
| | vhtssagtla |
| 1861 | tdrslntgvt rasrlengsd tsskslsmgn sthtsmtyte ksevsssihp |
| | rpetsapgae |
| 1921 | ttltstpgnr aisltlpfss ipveevistg itsgpdinsa pmthspitpp |
| | tivwtstgti |
| 1981 | eqstqplhav ssekvsvqtq stpyvnsvav saspthensv ssgsstsspy |
| | ssasleslds |
| 2041 | tisrrnaits wlwdlttslp tttwpstsls ealssghsgv snpsstttef |
| | plfsaastsa |
| 2101 | akqrnpetet hgpqntaast lntdassvtg lsetpvgasi ssevplpmai |
| | tsrsdvsglt |
| 2161 | sestanpslg tassagtklt rtislptses lvsfrmnkdp wtvsiplgsh |
| | pttntetsip |
| 2221 | vnsagppgls tvasdvidtp sdgaesiptv sfspspdtev ttishfpekt |
| | thsfrtissl |
| 2281 | theltsrvtp ipgdwmssam stkptgasps itlgerrtit saapttspiv |
| | ltasftetst |
| 2341 | vsldnettvk tsdildarkt nelpsdssss sdlintsias stmdvtktas |
| | isptsisgmt |
| 2401 | assspslfss drpqvptstt etntatspsv ssntysldgg snvggtpstl |
| | ppftithpve |
| 2461 | tssallawsr pvrtfstmvs tdtasgenpt ssnsvvtsvp apgtwtsvgs |
| | ttdlpamgfl |
| 2521 | ktspageahs llastiepat aftphlsaav vtgssatsea sllttseska |
| | ihsspqtptt |
| 2581 | ptsganwets atpesllvvt etsdttltsk ilvtdtilfs tvstppskfp |
| | stgtlsgasf |
| 2641 | ptllpdtpai pltateptss latsfdstpl vtiasdslgt vpettltmse |
| | tsngdalvlk |
| 2701 | tvsnpdrsip gitiqgvtes plhpsstsps kivaprntty egsitvalst |
| | lpagttgslv |
| 2761 | fsqssenset talvdssagl erasvmpltt gsqgmassgg irsgsthstg |
| | tktfsslplt |
| 2821 | mnpgevtams eittnrltat qstapkgipv kptsaesgll tpvsasssps |
| | kafaslttap |
| 2881 | ptwgipqstl tfefsevpsl dtksaslptp gqslntipds dastasssls |
| | kspeknprar |
| 2941 | mmtstkaisa ssfqstgfte tpegsaspsm agheprvpts gtgdpryase |
| | smsypdpska |
| 3001 | ssamtstsla sklttlfstg qaarsgssss pislsteket sflsptasts |
| | rktslflgps |
| 3061 | marqpnilvh lqtsaltlsp tstlnmsqee ppeltssqti aeeegttaet |
| | qtltftpset |
| 3121 | ptsllpvssp teptarrkss petwassisv paktslvett dgtlvttikm |
| | ssqaaqgnst |
| 3181 | wpapaeetgs spagtspgsp emsttlkims skepsispei rstvrnspwk |
| | tpettvpmet |
| 3241 | tvepvtlqst algsgstsis hlptgttspt ksptenmlat ervslspspp |
| | eawtnlysgt |
| 3301 | pggtrqslat mssvslespt arsitgtgqq sspelvsktt gmefsmwhgs |
| | tggttgdthv |
| 3361 | slstssnile dpvtspnsvs sltdkskhkt etwvsttaip stvlnnkima |
| | aeqqtsrsvd |
| 3421 | eaysstssws dqtsgsditl gaspdvtntl yitstaqtts lvslpsgdqg |
| | itsltnpsgg |
| 3481 | ktssassvts psigletlra nvsavksdia ptaghlsqts spaevsildv |
| | ttaptpgist |
| 3541 | tittmgtnsi stttpnpevg mstmdstpat errttstehp stwsstaasd |
| | swtvtdmtsn |
| 3601 | lkvarspgti stmhttsfla ssteldsmst phgritvigt slvtpssdas |
| | avktetstse |
| 3661 | rtlspsdtta stpistfsrv qrmsisvpdi lstswtpsst eaedvpvsmv |
| | stdhastktd |
| 3721 | pntplstflf dslstldwdt grslssatat tsapqgattp qeltletmis |
| | patsqlpfsi |
| 3781 | ghitsavtpa amarssgvtf srpdptskka eqtstqlptt tsahpgqvpr |
| | saattldvip |
| 3841 | htaktpdatf qrqgqtaltt earatsdswn ekekstpsap witemmnsvs |
| | edtikevtss |
| 3901 | ssvlrtlntl dinlesgtts spswksspye riapsesttd keaihpstnt |
| | vettgwvtss |
| 3961 | ehashstipa hsasskltsp vvttstreqa ivsmstttwp estrartepn |
| | sfltielrdv |
| 4021 | spymdtsstt qtsiisspgs taitkgprte itsskrisss flaqsmrssd |
| | spseaitrls |
| 4081 | nfpamtesgg milamqtspp gatslsaptl dtsataswtg tplattqrft |
| | ysekttlfsk |
| 4141 | gpedtsqpsp psveetssss slvpihatts psnilltsqg hspsstppvt |
| | svflsetsgl |
| 4201 | gkttdmsris lepgtslppn lsstageals tyeasrdtka ihhsadtavt |
| | nmeatsseys |
| 4261 | pipghtkpsk atsplvtshi mgditsstsv fgssetteie tvssvnqglq |
| | erstsqvass |
| 4321 | atetstvith vssgdatthv tktqatfssg tsissphqfi tstntftdvs |
| | tnpstslimt |
| 4381 | essgvtittq tgptgaatqg pylldtstmp yltetplavt pdfmqsektt |
| | liskgpkdvs |
| 4441 | wtsppsvaet sypssltpfl vttippatst lqgqhtsspv satsvltsgl |
| | vkttdmlnts |
| 4501 | mepvtnspqn lnnpsneila tlaattdiet ihpsinkavt nmgtassahv |
| | lhstlpvsse |
| 4561 | pstatspmvp assmgdalas isipgsettd iegeptsslt agrkenstlq |
| | emnsttesni |
| 4621 | ilsnvsvgai teatkmevps fdatfiptpa qstkfpdifs vassrlsnsp |
| | pmtisthmtt |
| 4681 | tqtgssgats kiplaldtst letsagtpsv vtegfahski ttamnndvkd |
| | vsqtnppfqd |
| 4741 | easspssqap vlvttlpssv aftpqwhsts spvsmssvlt sslvktagkv |
| | dtsletvtss |
| 4801 | pqsmsntldd isvtsaattd ietthpsint vvtnvgttgs afeshstvsa |
| | ypepskvtsp |
| 4861 | nvttstmedt tisrsipkss kttrtetett ssltpklret sisqeitsst |
| | etstvpykel |
| 4921 | tgattevsrt dvtsssstsf pgpdqstvsl distetntrl stspimtesa |
| | eitittqtgp |
| 4981 | hgatsqdtft mdpsnttpqa gihsamthgf sqldvttlms ripqdvswts |
| | ppsvdktssp |
| 5041 | ssflsspamt tpslisstlp edklsspmts lltsglvkit dilrtrlepv |
| | tsslpnfsst |
| 5101 | sdkilatskd skdtkeifps inteetnvka nnsgheshsp aladsetpka |
| | ttqmvitttv |
| 5161 | gdpapstsmp vhgssettni kreptyfltp rlretstsqe ssfptdtsfl |
| | lskvptgtit |
| 5221 | evsstgvnss skistpdhdk stvppdtftg eiprvftssi ktksaemtit |
| | tqasppesas |
| 5281 | hstlpldtst tlsqggthst vtqgfpysev ttlmgmgpgn vswmttppve |
| | etssvsslms |
| 5341 | spamtspspv sstspqsips splpvtalpt svlvtttdvl gttspesvts |
| | sppnlssith |
| 5401 | erpatykdta hteaamhhst ntavtnvgts gsghksqssv ladsetskat |
| | plmsttstlg |
| 5461 | dtsvststpn isqtnqiqte ptaslsprlr esstsektss ttetntafsy |
| | vptgaitqas |
| 5521 | rteisssrts isdldrptia pdistgmitr lftspimtks aemtvttqtt |
| | tpgatsqgil |
| 5581 | pwdtsttlfq ggthstvsqg fphseittlr srtpgdvswm ttppveetss |
| | gfslmspsmt |
| 5641 | spspvsstsp esipssplpv talltsvlvt ttnvlgttsp epvtssppnl |
| | ssptqerltt |
| 5701 | ykdtahteam hasmhtntav anvgtsisgh esqssvpads htskatspmg |
| | itfamgdtsv |
| 5761 | ststpaffet riqtestssl ipglrdtrts eeintvtets tvlsevpttt |
| | ttevsrtevi |
| 5821 | tssrttisgp dhskmspyis tetitrlstf pfvtgstema itnqtgpigt |
| | isqatltldt |
| 5881 | sstaswegth spvtqrfphs eetttmsrst kgvswqspps veetsspssp |
| | vplpaitshs |
| 5941 | slysavsgss ptsalpvtsl ltsgrrktid mldthselvt sslpsassfs |
| | geiltseast |
| 6001 | ntetihfsen taetnmgttn smhklhssvs ihsqpsghtp pkvtgsmmed |
| | aivststpgs |
| 6061 | petknvdrds tspltpelke dstalvmnst tesntvfssv sldaatevsr |
| | aevtyydptf |
| 6121 | mpasaqstks pdispeasss hsnsppltis thktiatqtg psgvtslgql |
| | tldtstiats |
| 6181 | agtpsartqd fvdsettsvm nndlndvlkt spfsaeeans lssqapllvt |
| | tspspvtstl |
| 6241 | qehstsslvs vtsvptptla kitdmdtnle pvtrspqnlr ntlatseatt |
| | dthtmhpsin |
| 6301 | tavanvgtts spnefyftvs pdsdpykats avvitstsgd sivstsmprs |
| | samkkieset |
| 6361 | tfslifrlre tstsqkigss sdtstvfdka ftaattevsr teltsssrts |
| | iqgtekptms |
| 6421 | pdtstrsvtm lstfagltks eertiatqtg phratsqgtl twdtsittsq |
| | agthsamthg |
| 6481 | fsqldlstlt srvpeyisgt sppsvektss sssllslpai tspspvpttl |
| | pesrpsspvh |
| 6541 | ltslptsglv kttdmlasva slppnlgsts hkipttsedi kdtekmypst |
| | niavtnvgtt |
| 6601 | tsekesyssv payseppkvt spmvtsfnir dtivstsmpg sseitrieme |
| | stfslahglk |
| 6661 | gtstsqdpiv steksavlhk lttgatetsr tevassrrts ipgpdhstes |
| | pdistevips |
| 6721 | lpislgites snmtiitrtg pplgstsqgt ftldtpttss ragthsmatq |
| | efphsemttv |
| 6781 | mnkdpeilsw tippsiekts fssslmpspa mtsppvsstl pktihttpsp |
| | mtslltpslv |
| 6841 | mttdtlgtsp epttssppnl sstsheiltt dedttaieam hpststaatn |
| | vettssghgs |
| 6901 | qssvladsek tkatapmdtt stmghttvst smsvssettk ikrestyslt |
| | pglretsisq |
| 6961 | nasfstdtsi vlsevptgtt aevsrtevts sgrtsipgps qstvlpeist |
| | rtmtrlfasp |
| 7021 | tmtesaemti ptqtgpsgst sqdtltldts ttksqakths tltqrfphse |
| | mttlmsrgpg |
| 7081 | dmswqsspsl enpsslpsll slpattsppp isstlpvtis ssplpvtsll |
| | tsspvtttdm |
| 7141 | lhtspelvts sppklshtsd erlttgkdtt nteavhpstn taasnveips |
| | sghespssal |
| 7201 | adsetskats pmfitstqed ttvaistphf letsriqkes isslspklre |
| | tgssvetssa |
| 7261 | ietsavlsev sigatteisr tevtsssrts isgsaestml peisttrkii |
| | kfptspilae |
| 7321 | ssemtiktqt sppgstsest ftldtsttps lvithstmtq rlphseittl |
| | vsrgagdvpr |
| 7381 | psslpveets ppssqlslsa mispspvsst lpasshsssa svtslltpgq |
| | vkttevldas |
| 7441 | aepetsspps lsstsveila tsevttdtek ihpfsntavt kvgtsssghe |
| | spssvlpdse |
| 7501 | ttkatsamgt isimgdtsvs tltpalsntr kiqsepassl ttrlretsts |
| | eetslatean |
| 7561 | tvlskvstga ttevsrteai sfsrtsmsgp eqstmsqdis igtiprisas |
| | svltesakmt |
| 7621 | ittqtgpses tlestlnlnt attpswveth siviqgfphp emttsmgrgp |
| | ggvswpsppf |
| 7681 | vketsppssp lslpavtsph pvsttflahi ppsplpvtsl ltsgpatttd |
| | ilgtstepgt |
| 7741 | ssssslstts herlttykdt ahteavhpst ntggtnvatt ssgyksqssv |
| | ladsspmctt |
| 7801 | stmgdtsvlt stpafletrr iqtelasslt pglressgse gtssgtkmst |
| | vlskvptgat |
| 7861 | teiskedvts ipgpaqstis pdistrtvsw fstspvmtes aeitmnthts |
| | plgattqgts |
| 7921 | tldtssttsl tmthstisqg fshsqmstlm rrgpedvswm sppllektrp |
| | sfslmsspat |
| 7981 | tspspvsstl pesisssplp vtslltsgla kttdmlhkss epvtnspanl |
| | sstsveilat |
| 8041 | sevttdtekt hpssnrtvtd vgtsssghes tsfvladsqt skvtspmvit |
| | stmedtsvst |
| 8101 | stpgffetsr iqteptsslt lglrktssse gtslatemst vlsgvptgat |
| | aevsrtevts |
| 8161 | ssrtsisgfa qltvspetst etitrlptss imtesaemmi ktqtdppgst |
| | pesthtvdis |
| 8221 | ttpnwveths tvtqrfshse mttlvsrspg dmlwpsqssv eetssassll |
| | slpattspsp |
| 8281 | vsstlvedfp saslpvtsll npglvittdr mgisrepgts stsnlsstsh |
| | erlttledtv |
| 8341 | dtedmqpsth tavtnvrtsi sghesqssvl sdsetpkats pmgttytmge |
| | tsvsistsdf |
| 8401 | fetsriqiep tssltsglre tssserissa tegstvlsev psgattevsr |
| | tevissrgts |
| 8461 | msgpdqftis pdisteaitr lstspimtes aesaitietg spgatsegtl |
| | tldtstttfw |
| 8521 | sgthstaspg fshsemttlm srtpgdvpwp slpsveeass vssslsspam |
| | tstsffstlp |
| 8581 | esisssphpv talltlgpvk ttdmlrtsse petssppnls stsaeilats |
| | evtkdrekih |
| 8641 | pssntpvvnv gtviykhlsp ssvladlvtt kptspmatts tlgntsvsts |
| | tpafpetmmt |
| 8701 | qptssltsgl reistsqets satersasls gmptgattkv srtealslgr |
| | tstpgpaqst |
| 8761 | ispeisteti tristplttt gsaemtitpk tghsgassqg tftldtssra |
| | swpgthsaat |
| 8821 | hrsphsgmtt pmsrgpedvs wpsrpsvekt sppsslvsls avtspsplys |
| | tpsesshssp |
| 8881 | lrvtslftpv mmkttdmldt slepvttspp smnitsdesl atskatmete |
| | aiqlsentav |
| 8941 | tqmgtisarq efyssypglp epskvtspvv tsstikdivs ttipasseit |
| | riemeststl |
| 9001 | tptpretsts qeihsatkps tvpykaltsa tiedsmtqvm sssrgpspdq |
| | stmsqdiste |
| 9061 | vitrlstspi ktestemtit tqtgspgats rgtltldtst tfmsgthsta |
| | sqgfshsqmt |
| 9121 | almsrtpgdv pwlshpsvee assasfslss pvmtssspvs stlpdsihss |
| | slpvtsllts |
| 9181 | glvkttellg tssepetssp pnlsstsaei laitevttdt eklemtnvvt |
| | sgythespss |
| 9241 | vladsvttka tssmgitypt gdtnvltstp afsdtsriqt ksklsltpgl |
| | metsiseets |
| 9301 | satekstvls svptgattev srteaisssr tsipgpaqst mssdtsmeti |
| | tristpltrk |
| 9361 | estdmaitpk tgpsgatsqg tftldsssta swpgthsatt qrfpqsvvtt |
| | pmsrgpedvs |
| 9421 | wpsplsvekn sppsslvsss svtspsplys tpsgsshssp vpvtslftsi |
| | mmkatdmlda |
| 9481 | slepettsap nmnitsdesl aaskattete aihvfentaa shvettsate |
| | elyssspgfs |
| 9541 | eptkvispvv tsssirdnmv sttmpgssgi trieiesmss ltpglretrt |
| | sqditsstet |
| 9601 | stvlykmpsg atpevsrtev mpssrtsipg paqstmsldi sdevvtrlst |
| | spimtesaei |
| 9661 | tittqtgysl atsqvtlplg tsmtflsgth stmsqglshs emtnlmsrgp |
| | eslswtsprf |
| 9721 | vettrssssl tslplttsls pvsstlldss pssplpvtsl ilpglvktte |
| | vldtssepkt |
| 9781 | ssspnlssts veipatseim tdtekihpss ntavakvrts ssvheshssv |
| | ladsettiti |
| 9841 | psmgitsavd dttvftsnpa fsetrripte ptfsltpgfr etstseetts |
| | itetsavlyg |
| 9901 | vptsattevs mteimssnri hipdsdqstm spdiitevit rlssssmmse |
| | stqmtittqk |
| 9961 | sspgataqst ltlatttapl arthstvppr flhsemttlm srspenpswk |
| | sslfvektss |
| 10021 | sssllslpvt tspsvsstlp qsipsssfsv tslltpgmvk ttdtstepgt |
| | slspnlsgts |
| 10081 | veilaasevt tdtekihpss smavtnvgtt ssghelyssv sihsepskat |
| | ypvgtpssma |
| 10141 | etsistsmpa nfettgfeae pfshltsgfr ktnmsldtss vtptntpssp |
| | gsthllqssk |
| 10201 | tdftssakts spdwppasqy teipvdiitp fnaspsites tgitsfpesr |
| | ftmsvtesth |
| 10261 | hlstdllpsa etistgtvmp slseamtsfa ttgvpraisg sgspfsrtes |
| | gpgdatlsti |
| 10321 | aeslpsstpv pfssstfttt dsstipalhe itsssatpyr vdtslgtess |
| | ttegrlvmvs |
| 10381 | tldtssqpgr tssspildtr mtesvelgtv tsayqvpsls trltrtdgim |
| | ehitkipnea |
| 10441 | ahrgtirpvk gpqtstspas pkglhtggtk rmettttalk ttttalktts |
| | ratlttsvyt |
| 10501 | ptlgtltpln asmqmastip temmittpyv fpdvpettss latslgaets |
| | talprttpsv |
| 10561 | fnresettas lvsrsgaers pviqtldvss sepdttaswv ihpaetiptv |
| | skttpnffhs |
| 10621 | eldtvsstat shgadvssai ptnispseld altplvtisg tdtsttfptl |
| | tksphetetr |
| 10681 | ttwlthpaet sstiprtipn fshhesdatp siatspgaet ssaipimtvs |
| | pgaedlvtsq |
| 10741 | vtssgtdrnm tiptltlspg epktiaslvt hpeaqtssai ptstispavs |
| | rlvtsmvtsl |
| 10801 | aaktsttnra ltnspgepat tvslvthpaq tsptvpwtts iffhsksdtt |
| | psmttshgae |
| 10861 | sssavptptv stevpgvvtp lvtssravis ttipiltlsp gepettpsma |
| | tshgeeassa |
| 10921 | iptptvspgv pgvvtslvts sravtsttip iltfslgepe ttpsmatshg |
| | teagsavptv |
| 10981 | lpevpgmvts lvassravts ttlptltlsp gepettpsma tshgaeasst |
| | vptvspevpg |
| 11041 | vvtslvtsss gvnstsiptl ilspgelett psmatshgae assavptptv |
| | spgvsgvvtp |
| 11101 | lvtssravts ttipiltlss sepettpsma tshgveassa vltvspevpg |
| | mvtslvtssr |
| 11161 | avtsttiptl tissdepett tslvthseak misaiptlav sptvqglvts |
| | lvtssgsets |
| 11221 | afsnltvass qpetidswva hpgteassvv ptltvstgep ftnislvthp |
| | aessstlprt |
| 11281 | tsrfshseld tmpstvtspe aesssaistt ispgipgvlt slvtssgrdi |
| | satfptvpes |
| 11341 | pheseatasw vthpavtstt vprttpnysh sepdttpsia tspgaeatsd |
| | fptitvspdv |
| 11401 | pdmvtsqvts sgtdtsitip tltlssgepe tttsfityse thtssaiptl |
| | pvspgaskml |
| 11461 | tslvissgtd stttfptlte tpyepettai qlihpaetnt mvprttpkfs |
| | hsksdttlpv |
| 11521 | aitspgpeas savstttisp dmsdlvtslv pssgtdtstt fptlsetpye |
| | pettatwlth |
| 11581 | paetsttvsg tipnfshrgs dtapsmvtsp gvdtrsgvpt ttippsipgv |
| | vtsqvtssat |
| 11641 | dtstaiptlt pspgepetta ssathpgtqt gftvpirtvp ssepdtmasw |
| | vthppqtstp |
| 11701 | vsrttssfsh sspdatpvma tsprteassa vlttispgap emvtsqitss |
| | gaatsttvpt |
| 11761 | lthspgmpet tallsthprt etsktfpast vfpqvsetta sltirpgaet |
| | stalptqtts |
| 11821 | slftllvtgt srvdlsptas pgvsaktapl sthpgtetst miptstlslg |
| | llettgllat |
| 11881 | sssaetstst ltltvspavs glssasittd kpqtvtswnt etspsvtsvg |
| | ppefsrtvtg |
| 11941 | ttmtlipsem ptppktshge gvspttilrt tmveatnlat tgssptvakt |
| | tttfntlags |
| 12001 | lftplttpgm stlasesvts rtsynhrswi sttssynrry wtpatstpvt |
| | stfspgists |
| 12061 | sipsstaatv pfmvpftlnf titnlqyeed mrhpgsrkfn aterelqgll |
| | kplfrnssle |
| 12121 | ylysgcrlas lrpekdssat avdaicthrp dpedlgldre rlywelsnlt |
| | ngiqelgpyt |
| 12181 | ldrnslyvng fthrssmptt stpgtstvdv gtsgtpsssp spttagpllm |
| | pftlnftitn |
| 12241 | lqyeedmrrt gsrkfntmes vlqgllkplf kntsvgplys gcrltllrpe |
| | kdgaatgvda |
| 12301 | icthrldpks pglnreqlyw elskltndie elgpytldrn slyvngfthq |
| | ssvsttstpg |
| 12361 | tstvdlrtsg tpsslsspti maagpllvpf tlnftitnlq ygedmghpgs |
| | rkfnttervl |
| 12421 | qgllgpifkn tsvgplysgc rltslrsekd gaatgvdaic ihhldpkspg |
| | lnrerlywel |
| 12481 | sqltngikel gpytldrnsl yvngfthrts vptsstpgts tvdlgtsgtp |
| | fslpspatag |
| 12541 | pllvlftlnf titnlkyeed mhrpgsrkfn ttervlqtll gpmfkntsvg |
| | llysgcrltl |
| 12601 | lrsekdgaat gvdaicthrl dpkspgvdre qlywelsqlt ngikelgpyt |
| | ldrnslyvng |
| 12661 | fthwipvpts stpgtstvdl gsgtpsslps pttagpllvp ftlnftitnl |
| | kyeedmhcpg |
| 12721 | srkfntterv lqsllgpmfk ntsvgplysg crltllrsek dgaatgvdai |
| | cthrldpksp |
| 12781 | gvdreqlywe lsqltngike lgpytldrns lyvngfthqt sapntstpgt |
| | stvdlgtsgt |
| 12841 | psslpsptsa gpllvpftln ftitnlqyee dmhhpgsrkf nttervlqgl |
| | lgpmfkntsv |
| 12901 | gllysgcrlt llrpekngaa tgmdaicshr ldpkspglnr eqlywelsql |
| | thgikelgpy |
| 12961 | tldrnslyvn gfthrssvap tstpgtstvd lgtsgtpssl pspttavpll |
| | vpftlnftit |
| 13021 | nlqygedmrh pgsrkfntte rvlqgllgpl fknssvgply sgcrlislrs |
| | ekdgaatgvd |
| 13081 | aicthhlnpq spgldreqly wqlsqmtngi kelgpytldr nslyvngfth |
| | rssglttstp |
| 13141 | wtstvdlgts gtpspvpspt ttgpllvpft lnftitnlqy eenmghpgsr |
| | kfnitesvlq |
| 13201 | gllkplfkst svgplysgcr ltllrpekdg vatrvdaict hrpdpkipgl |
| | drqqlywels |
| 13261 | qlthsitelg pytldrdsly vngftqrssv pttstpgtft vqpetsetps |
| | slpgptatgp |
| 13321 | vllpftlnft itnlqyeedm rrpgsrkfnt tervlqgllm plfkntsvss |
| | lysgcrltll |
| 13381 | rpekdgaatr vdavcthrpd pkspgldrer lywklsqlth gitelgpytl |
| | drhslyvngf |
| 13441 | thqssmtttr tpdtstmhla tsrtpaslsg pmtaspllvl ftinftitnl |
| | ryeenmhhpg |
| 13501 | srkfntterv lqgllrpvfk ntsvgplysg crltllrpkk dgaatkvdai |
| | ctyrpdpksp |
| 13561 | gldreqlywe lsqlthsite lgpytldrds lyvngftqrs svpttsipgt |
| | ptvdlgtsgt |
| 13621 | pvskpgpsaa spllvlftln ftitnlryee nmqhpgsrkf nttervlqgl |
| | lrslfkstsv |
| 13681 | gplysgcrlt llrpekdgta tgvdaicthh pdpksprldr eqlywelsql |
| | thnitelgpy |
| 13741 | aldndslfvn gfthrssvst tstpgtptvy lgasktpasi fgpsaashll |
| | ilftlnftit |
| 13801 | nlryeenmwp gsrkfntter vlqgllrplf kntsvgplys gcrltllrpe |
| | kdgeatgvda |
| 13861 | icthrpdptg pgldreqlyl elsqlthsit elgpytldrd slyvngfthr |
| | ssvpttstgv |
| 13921 | vseepftlnf tinnlrymad mgqpgslkfn itdnvmqhll splfqrsslg |
| | arytgcrvia |
| 13981 | lrsvkngaet rvdllctylq plsgpglpik qvfhelsqqt hgitrlgpys |
| | ldkdslylng |
| 14041 | ynepgpdepp ttpkpattfl pplseattam gyhlktltln ftisnlqysp |
| | dmgkgsatfn |
| 14101 | stegvlqhll rplfqkssmg pfylgcqlis lrpekdgaat gvdttctyhp |
| | dpvgpgldiq |
| 14161 | qlywelsqlt hgvtqlgfyv ldrdslfing yapqnlsirg eyqinfhivn |
| | wnlsnpdpts |
| 14221 | seyitllrdi qdkvttlykg sqlhdtfrfc lvtnltmdsv lvtvkalfss |
| | nldpslveqv |
| 14281 | fldktlnasf hwlgstyqlv dihvtemess vyqptsssst qhfylnftit |
| | nlpysqdkaq |
| 14341 | pgttnyqrnk rniedalnql frnssiksyf sdcqvstfrs vpnrhhtgvd |
| | slcnfsplar |
| 14401 | rvdrvaiyee flrmtrngtq lqnftldrss vlvdgyspnr nepltgnsdl |
| | pfwaviligl |
| 14461 | agllgvitcl icgvlvttrr rkkegeynvq qqcpgyyqsh ldledlq |

By “HE4” is meant a polypeptide biomarker having at least 85% sequence identity to NCBI accession numbers AA052683 or CAA44869 or a fragment thereof. An exemplary sequence of HE4 is:

| | |
|---|---|
| 1 | mpacrlgpla aalllslllf gftlvsgtga ektgvcpelq |
| | adqnctqecv sdsecadnlk |
| 61 | ccsagcatfc slpndkegsc pqvninfpql |
| | glcrdqcqvd sqcpgqmkcc rngcgkvscv |
| 121 | tpnf |

By “IL-2 receptor alpha (IL-2Rα)” is meant a polypeptide biomarker having at least 85% sequence identity to NCBI accession numbers CAK26553 or NP_{—}000408 or a fragment thereof. An exemplary sequence of IL-2Rα is:

| | |
|---|---|
| 1 | mdsyllmwgl ltfimvpgcq aelcdddppe iphatfkama |
| | ykegtmlnce ckrgfrriks |
| 61 | gslymlctgn sshsswdnqc qctssatrnt tkqvtpqpee |
| | qkerkttemq spmqpvdqas |
| 121 | lpghcreppp weneateriy hfvvgqmvyy qcvqgyralh |
| | rgpaesvckm thgktrwtqp |
| 181 | qlictgemet sqfpgeekpq aspegrpese tsclvtttdf |
| | qiqtemaatm etsiftteyq |
| 241 | vavagcvfll isvlllsglt wqrrqrksrr ti |

By “Alpha-1-Antitrypsin (AAT)” is meant a polypeptide biomarker having at least 85% sequence identity to NCBI accession numbers AAB59495 or CAJ15161 or a fragment thereof. An exemplary sequence of ATT is:

| | |
|---|---|
| 1 | mpssvswgil llaglcclvp vslaedpqgd aaqktdtshh |
| | dqdhptfnki tpnlaefafs |
| 61 | lyrqlahqsn stniffspvs iatafamlsl gtkadthdei |
| | leglnfnlte ipeaqihegf |
| 121 | qellrtlnqp dsqlqlttgn glflseglkl vdkfledvkk |
| | lyhseaftvn fgdteeakkq |
| 181 | indyvekgtq gkivdlvkel drdtvfalvn yiffkgkwer |
| | pfevkdteee dfhvdqvttv |
| 241 | kvpmmkrlgm fniqhckkls swvllmkylg nataifflpd |
| | egklqhlvne lthdiitkfl |
| 301 | enedrrsasl hlpklsitgt ydlksvlgql gitkvfsnga |
| | dlsgvteeap lklskavhka |
| 361 | vltidekgte aagamfleai pmsippevkf nkpfvflmie |
| | qntksplfmg kvvnptqk |

By “C-Reactive Protein (CRP)” is meant a polypeptide biomarker having at least 85% sequence identity to NCBI accession numbers CAA39671 or P02741 or a fragment thereof. An exemplary sequence of CRP is:

| | |
|---|---|
| 1 | mekllcflvl tslshafgqt dmsrkafvfp kesdtsyvsl |
| | kapltkplka ftvclhfyte |
| 61 | lsstrgtvfs rmpprdktmr ffifwskdig ysftvggsei |
| | lfevpevtva pvhictswes |
| 121 | asgivefwvd gkprvrkslk kgytvgaeas iilgqeqdsf |
| | ggnfegsqsl vgdignvnmw |
| 181 | dfvlspdein tiylggpfsp nvlnwralky evqgevftkp |
| | qlwp |

By “YKL-40” also know as “chitinase-3-like protein” is meant a polypeptide biomarker having at least 85% sequence identity to NCBI accession numbers P36222 or NP_{—}001267 or a fragment thereof. An exemplary sequence of YKL-40 is:

| | |
|---|---|
| 1 | mgvkasqtgf vvlvllqccs ayklvcyyts wsqyregdgs |
| | cfpdaldrfl cthiiysfan |
| 61 | isndhidtwe wndvtlygml ntlknrnpnl ktllsvggwn |
| | fgsqrfskia sntqsrrtfi |
| 121 | ksvppflrth gfdgldlawl ypgrrdkqhf ttlikemkae |
| | fikeaqpgkk qlllsaalsa |
| 181 | gkvtidssyd iakisqhldf isimtydfhg awrgttghhs |
| | plfrgqedas pdrfsntdya |
| 241 | vgymlrlgap asklvmgipt fgrsftlass etgvgapisg |
| | pgipgrftke agtlayyeic |
| 301 | dflrgatvhr ilgqqvpyat kgnqwvgydd qesvkskvqy |
| | lkdrqlagam vwaldlddfq |
| 361 | gsfcgqdlrf pltnaikdal aat |

By “Cellular Fibronectin (cFib)” is meant a polypeptide biomarker having at least 85% sequence identity to NCBI accession numbers P02751 or a fragment thereof. An exemplary sequence of cFib is:

| | |
|---|---|
| 1 | mlrgpgpgll llavqclgta vpstgasksk rqaqqmvqpq |
| | spvavsqskp gcydngkhyq |
| 61 | inqqwertyl gnalvctcyg gsrgfncesk peaeetcfdk |
| | ytgntyrvgd tyerpkdsmi |
| 121 | wdctcigagr grisctianr cheggqsyki gdtwrrphet |
| | ggymlecvcl gngkgewtck |
| 181 | piaekcfdha agtsyvvget wekpyqgwmm vdctclgegs |
| | gritctsrnr cndqdtrtsy |
| 241 | rigdtwskkd nrgnllqcic tgngrgewkc erhtsvqtts |
| | sgsgpftdvr aavyqpqphp |
| 301 | qpppyghcvt dsgvvysvgm qwlktqgnkq mlctclgngv |
| | scqetavtqt yggnsngepc |
| 361 | vlpftyngrt fyscttegrq dghlwcstts nyeqdqkysf |
| | ctdhtvlvqt rggnsngalc |
| 421 | hfpflynnhn ytdctsegrr dnmkwcgttq nydadqkfgf |
| | cpmaaheeic ttnegvmyri |
| 481 | gdqwdkqhdm ghmmrctcvg ngrgewtcia ysqlrdqciv |
| | dditynvndt fhkrheeghm |
| 541 | lnctcfgqgr grwkcdpvdq cqdsetgtfy qigdswekyv |
| | hgvryqcycy grgigewhcq |
| 601 | plqtypsssg pvevfitetp sqpnshpiqw napqpshisk |
| | yilrwrpkns vgrwkeatip |
| 661 | ghlnsytikg lkpgvvyegq lisiqqyghq evtrfdfttt |
| | ststpvtsnt vtgettpfsp |
| 721 | lvatsesvte itassfvvsw vsasdtvsgf rveyelseeg |
| | depqyldlps tatsvnipdl |
| 781 | lpgrkyivnv yqisedgeqs lilstsqtta pdappdttvd |
| | qvddtsivvr wsrpqapitg |
| 841 | yrivyspsve gsstelnlpe tansvtlsdl qpgvqyniti |
| | yaveenqest pvviqqettg |
| 901 | tprsdtvpsp rdlqfvevtd vkvtimwtpp esavtgyrvd |
| | vipvnlpgeh gqrlpisrnt |
| 961 | faevtglspg vtyyfkvfav shgreskplt aqqttkldap |
| | tnlqfvnetd stvlvrwtpp |
| 1021 | raqitgyrlt vgltrrgqpr qynvgpsvsk yplrnlqpas |
| | eytvslvaik gnqespkatg |
| 1081 | vfttlqpgss ippyntevte ttivitwtpa prigfklgvr |
| | psqggeapre vtsdsgsivv |
| 1141 | sgltpgveyv ytiqvlrdgq erdapivnkv vtplspptnl |
| | hleanpdtgv ltvswerstt |
| 1201 | pditgyritt tptngqqgns leevvhadqs sctfdnlspg |
| | leynvsvytv kddkesvpis |
| 1261 | dtiipavppp tdlrftnigp dtmrvtwapp psidltnflv |
| | ryspvkneed vaelsispsd |
| 1321 | navvltnllp gteyvvsvss vyeqhestpl rgrqktglds |
| | ptgidfsdit ansftvhwia |
| 1381 | pratitgyri rhhpehfsgr predrvphsr nsitltnltp |
| | gteyvvsiva lngreespll |
| 1441 | igqqstvsdv prdlevvaat ptslliswda pavtvryyri |
| | tygetggnsp vqeftvpgsk |
| 1501 | statisglkp gvdytitvya vtgrgdspas skpisinyrt |
| | eidkpsqmqv tdvqdnsisv |
| 1561 | kwlpssspvt gyrvtttpkn gpgptktkta gpdqtemtie |
| | glqptveyvv svyaqnpsge |
| 1621 | sqplvqtavt nidrpkglaf tdvdvdsiki awespqgqvs |
| | ryrvtysspe dgihelfpap |
| 1681 | dgeedtaelq glrpgseytv svvalhddme sqpligtqst |
| | aipaptdlkf tqvtptslsa |
| 1741 | qwtppnvqlt gyrvrvtpke ktgpmkeinl apdsssvvvs |
| | glmvatkyev svyalkdtlt |
| 1801 | srpaqgvvtt lenvspprra rvtdatetti tiswrtktet |
| | itgfqvdavp angqtpiqrt |
| 1861 | ikpdvrsyti tglqpgtdyk iylytlndna rsspvvidas |
| | taidapsnlr flattpnsll |
| 1921 | vswqpprari tgyiikyekp gspprevvpr prpgvteati |
| | tglepgteyt iyvialknnq |
| 1981 | ksepligrkk tdelpqlvtl phpnlhgpei ldvpstvqkt |
| | pfvthpgydt gngiqlpgts |
| 2041 | gqqpsvgqqm ifeehgfrrt tppttatpir hrprpyppnv |
| | geeiqighip redvdyhlyp |
| 2101 | hgpglnpnas tgqealsqtt iswapfqdts eyiischpvg |
| | tdeeplqfrv pgtstsatlt |
| 2161 | gltrgatynv ivealkdqqr hkvreevvtv gnsvneglnq |
| | ptddscfdpy tvshyavgde |
| 2221 | wermsesgfk llcqclgfgs ghfrcdssrw chdngvnyki |
| | gekwdrqgen gqmmsctclg |
| 2281 | ngkgefkcdp heatcyddgk tyhvgeqwqk eylgaicsct |
| | cfggqrgwrc dncrrpggep |
| 2341 | spegttgqsy nqysqryhqr tntnvncpie cfmpldvqad |
| | redsre |

By “Cancer Antigen 72-4 (CA-72-4)” also referred to as “TAG-72” is meant a glycoprotein biomarker which is recognized by monoclonal antibody B72.3.

By “prostasin” is meant a polypeptide biomarker having at least 85% sequence identity to NCBI accession numbers AAB19071 or AAC41759 or a fragment thereof. An exemplary sequence of prostasin is:

| | |
|---|---|
| 1 | maqkgvlgpg qlgavailly lgllrsgtga egaeapcgva |
| | pqaritggss avagqwpwqv |
| 61 | sityegvhvc ggslvseqwv lsaahcfpse hhkeayevkl |
| | gahqldsyse dakvstlkdi |
| 121 | iphpsylqeg sqgdiallql srpitfsryi rpiclpaana |
| | sfpnglhctv tgwghvapsv |
| 181 | slltpkplqq levplisret cnclynidak peephfvqed |
| | mvcagyvegg kdacqgdsgg |
| 241 | plscpveglw yltgivswgd acgarnrpgv ytlassyasw |
| | iqskvtelqp rvvpqtqesq |
| 301 | pdsnlcgshl afssapaqgl lrpilflplg lalgllspwl |
| | seh |

By “Tissue Inhibitor of Metalloproteinases 1 (TIMP-1)” is meant a polypeptide biomarker having at least 85% sequence identity to NCBI accession numbers NP_{—}003245 or P01033 or a fragment thereof. An exemplary sequence of TIMP-1 is:

| | |
|---|---|
| 1 | mapfeplasg illllwliap sractcvpph pqtafcnsdl |
| | virakfvgtp evnqttlyqr |
| 61 | yeikmtkmyk gfqalgdaad irfvytpame svcgyfhrsh |
| | nrseefliag klqdgllhit |
| 121 | tcsfvapwns lslaqrrgft ktytvgceec tvfpclsipc |
| | klqsgthclw tdqllqgsek |
| 181 | gfqsrhlacl prepglctwq slrsqia |

By “Interleukin 8 (IL-8)” is meant a polypeptide biomarker having at least 85% sequence identity to NCBI accession numbers P10145 or AAH13615 or a fragment thereof. An exemplary sequence of IL-8 is:

| | |
|---|---|
| 1 | mtsklavall aaflisaalc egavlprsak elrcqcikty |
| | skpfhpkfik elrviesgph |
| 61 | canteiivkl sdgrelcldp kenwvqrvve kflkraens |

By “Matrix Metalloproteinase-7 (MMP-7)” is meant a polypeptide biomarker having at least 85% sequence identity to NCBI accession numbers P09237 or NP_{—}002414 or a fragment thereof. An exemplary sequence of MMP-7 is:

| | |
|---|---|
| 1 | mrltvlcavc llpgslalpl pqeaggmsel qweqaqdylk |
| | rfylydsetk nansleaklk |
| 61 | emqkffglpi tgmlnsrvie imqkprcgvp dvaeyslfpn |
| | spkwtskvvt yrivsytrdl |
| 121 | phitvdrlvs kalnmwgkei plhfrkvvwg tadimigfar |
| | gahgdsypfd gpgntlahaf |
| 181 | apgtglggda hfdederwtd gsslginfly aathelghsl |
| | gmghssdpna vmyptygngd |
| 241 | pqnfklsqdd ikgiqklygk rsnsrkk |

By “Interleukin 6 (IL-6)” is meant a polypeptide biomarker having at least 85% sequence identity to NCBI accession numbers P05231, NP_{—}000591, or AAH15511 or a fragment thereof. An exemplary sequence of IL-6 is:

| | |
|---|---|
| 1 | mnsfstsafg pvafslglll vlpaafpapv ppgedskdva |
| | aphrqpltss eridkqiryi |
| 61 | ldgisalrke tcnksnmces skealaennl nlpkmaekdg |
| | cfqsgfneet clvkiitgll |
| 121 | efevyleylq nrfesseeqa ravqmstkvl iqflqkkakn |
| | ldaittpdpt tnaslltklq |
| 181 | aqnqwlqdmt thlilrsfke flqsslralr qm |

By “Vascular Endothelial Growth Factor B (VEGF-B)” is meant a polypeptide biomarker having at least 85% sequence identity to NCBI accession numbers P49765, AAC50721, or AAB06274 or a fragment thereof. An exemplary sequence of VEGF-B is:

| | |
|---|---|
| 1 | mspllrrlll aallqlapaq apvsqpdapg hqrkvvswid |
| | vytratcqpr evvvpltvel |
| 61 | mgtvakqlvp scvtvqrcgg ccpddglecv ptgqhqvrmq |
| | ilmirypssq lgemsleehs |
| 121 | qcecrpkkkd savkpdraat phhrpqprsv pgwdsapgap |
| | spadithptp apgpsahaap |
| 181 | sttsaltpgp aaaaadaaas svakgga |

By “calprotectin” is meant a polypeptide biomarker having at least 85% sequence identity to NCBI accession numbers AAB33355, AAB25118, or P06702 or a fragment thereof. An exemplary sequence of calprotectin is:

| | |
|---|---|
| 1 | mtckmsqler nietiintfh qysvklghpd tlnqgefkel |
| | vrkdlqnflk kenknekvie |
| 61 | himedldtna dkqlsfeefi mlmarltwas hekmhegdeg |
| | pghhhkpglg egtp |

By “Insulin-like Growth Factor-Binding Protein 2 (IGFBP-2)” is meant a polypeptide biomarker having at least 85% sequence identity to NCBI accession numbers AAA03246 or AAA36048 or a fragment thereof. An exemplary sequence of IGFBP-2 is:

| | |
|---|---|
| 1 | mlprvgcpal plppppllpl lpllllllga sgggggarae |
| | vlfrcppctp erlaacgppp |
| 61 | vappaavaav aggarmpcae lvrepgcgcc svcarlegea |
| | cgvytprcgq glrcyphpgs |
| 121 | elplqalvmg egtcekrrda eygaspeqva dngddhsegg |
| | lvenhvdstm nmlggggsag |
| 181 | rkplksgmke lavfrekvte qhrqmgkggk hhlgleepkk |
| | lrpppartpc qqeldqvler |
| 241 | istmrlpder gplehlyslh ipncdkhgly nlkqckmsln |
| | gqrgecwcvn pntgkliqga |
| 301 | ptirgdpech lfyneqqear gvhtqrmq |

By “Lectin-Like Oxidized LDL Receptor 1 (LOX-1)” is meant a polypeptide biomarker having at least 85% sequence identity to NCBI accession numbers P78380 or NP_{—}002534 or a fragment thereof. An exemplary sequence of LOX-1 is:

| | |
|---|---|
| 1 | mtfddlkiqt vkdqpdeksn gkkakglqfl yspwwclaaa |
| | tlgvlclglv vtimvlgmql |
| 61 | sqvsdlltqe qanlthqkkk legqisarqq aeeasqesen |
| | elkemietla rklnekskeq |
| 121 | melhhqnlnl qetlkrvanc sapcpqdwiw hgencylfss |
| | gsfnweksqe kclsldakll |
| 181 | kinstadldf iqqaisyssf pfwmglsrrn psypwlwedg |
| | splmphlfrv rgavsqtyps |
| 241 | gtcayiqrga vyaencilaa fsicqkkanl raq |

By “neuropilin-1” is meant a polypeptide biomarker having at least 85% sequence identity to NCBI accession numbers AAP80144, AAP78927, AAG41895, or ABY87548 or a fragment thereof. An exemplary sequence of neuropilin-1 is:

| | |
|---|---|
| 1 | merglpllca vlalvlapag afrndkcgdt ikiespgylt |
| | spgyphsyhp sekcewliqa |
| 61 | pdpyqrimin fnphfdledr dckydyvevf dgenenghfr |
| | gkfcgkiapp pvvssgpflf |
| 121 | ikfvsdyeth gagfsiryei fkrgpecsqn yttpsgviks |
| | pgfpekypns lectyivfap |
| 181 | kmseiilefe sfdlepdsnp pggmfcrydr leiwdgfpdv |
| | gphigrycgq ktpgrirsss |
| 241 | gilsmvfytd saiakegfsa nysvlqssvs edfkcmealg |
| | mesgeihsdq itassqystn |
| 301 | wsaersrlny pengwtpged syrewiqvdl gllrfvtavg |
| | tqgaisketk kkyyvktyki |
| 361 | dvssngedwi tikegnkpvl fqgntnptdv vvavfpkpli |
| | trfvrikpat wetgismrfe |
| 421 | vygckitdyp csgmlgmvsg lisdsqitss nqgdrnwmpe |
| | nirlvtsrsg walppaphsy |
| 481 | inewlqidlg eekivrgiii qggkhrenkv fmrkfkigys |
| | nngsdwkmim ddskrkaksf |
| 541 | egnnnydtpe lrtfpalstr firiyperat hgglglrmel |
| | lgceveapta gpttpngnlv |
| 601 | decdddqanc hsgtgddfql tggttvlate kptvidstiq |
| | sefptygfnc efgwgshktf |
| 661 | chwehdnhvq lkwsvltskt gpiqdhtgdg nfiysqaden |
| | qkgkvarlvs pvvysqnsah |
| 721 | cmtfwyhmsg shvgtlrvkl ryqkpeeydq lvwmaighqg |
| | dhwkegrvll hkslklyqvi |
| 781 | fegeigkgnl ggiavddisi nnhisqedca kpadldkknp |
| | eikidetgst pgyegegegd |
| 841 | knisrkpgnv lktldpilit iiamsalgvl lgavcgvvly |
| | cacwhngmse rnlsalenyn |
| 901 | felvdgvklk kdklntqsty sea |

By “TNFR2” is meant a polypeptide biomarker having at least 85% sequence identity to NCBI accession numbers P20333 or NP_{—}001057 or a fragment thereof. An exemplary sequence of TNFR2 is:

| | |
|---|---|
| 1 | mapvavwaal avglelwaaa halpaqvaft pyapepgstc |
| | rlreyydqta qmccskcspg |
| 61 | qhakvfctkt sdtvcdsced stytqlwnwv peclscgsrc |
| | ssdqvetqac treqnrictc |
| 121 | rpgwycalsk qegcrlcapl rkcrpgfgva rpgtetsdvv |
| | ckpcapgtfs nttsstdicr |
| 181 | phqicnvvai pgnasmdavc tstsptrsma pgavhlpqpv |
| | strsqhtqpt pepstapsts |
| 241 | fllpmgpspp aegstgdfal pvglivgvta lglliigvvn |
| | cvimtqvkkk plclqreakv |
| 301 | phlpadkarg tqgpeqqhll itapssssss lessasaldr |
| | raptrnqpqa pgveasgage |
| 361 | arastgssds spgghgtqvn vtcivnvcss sdhssqcssq |
| | asstmgdtds spsespkdeq |
| 421 | vpfskeecaf rsqletpetl lgsteekplp lgvpdagmkp s |

By “MPIF-1” is meant a polypeptide biomarker having at least 85% sequence identity to NCBI accession numbers AAB51134 or P55773 or a fragment thereof. An exemplary sequence of MPIF-1 is:

| | |
|---|---|
| 1 | mkvsvaalsc lmlvtalgsq arvtkdaete fmmsklplen |
| | pvlldrfhat sadccisytp |
| 61 | rsipcslles yfetnsecsk pgvifltkkg rrfcanpsdk |
| | qvqvcmrmlk ldtriktrkn |

### DETAILED DESCRIPTION

The biomarker panels and associated methods and products were identified through the analysis of analyte levels of various molecular species in human blood serum drawn from subjects having ovarian cancer of various stages and subtypes, subjects having non-cancer gynecological disorders and normal subjects. The immunoassays described below were courteously performed by our colleagues at Rules-Based Medicine of Austin, Tex. using their Multi-Analyte Profile (MAP) Luminex® platform.

While a preferred sample is blood serum, it is contemplated that an appropriate sample can be derived from any biological source or sample, such as tissues, extracts, cell cultures, including cells (for example, tumor cells), cell lysates, and physiological fluids, such as, for example, whole blood, plasma, serum, saliva, ductal lavage, ocular lens fluid, cerebral spinal fluid, sweat, urine, milk, ascites fluid, synovial fluid, peritoneal fluid and the like. The sample can be obtained from animals, preferably mammals, more preferably primates, and most preferably humans using species specific binding agents that are equivalent to those discussed below in the context of human sample analysis. It is further contemplated that these techniques and marker panels may be used to evaluate drug therapy in rodents and other animals, including transgenic animals, relevant to the development of human and veterinary therapeutics.

The sample can be treated prior to use by conventional techniques, such as preparing plasma from blood, diluting viscous fluids, and the like. Methods of sample treatment can involve filtration, distillation, extraction, concentration, inactivation of interfering components, addition of chaotropes, the addition of reagents, and the like. Nucleic acids (including silencer, regulatory and interfering RNA) may be isolated and their levels of expression for the analytes described below also used in the methods of the invention.

### Samples and Analytical Platform.

The set of blood serum samples that was analyzed to generate most of the data discussed below contained 150 ovarian cancer samples and 150 non-ovarian cancer samples. The ovarian cancer sample samples further comprised the following epithelial ovarian cancer subtypes: serous (64), clear cell (22), endometrioid (35), mucinous (15), mixed, that is, consisting of more than one subtype (14). The stage distribution of the ovarian cancer samples was: Stage I (41), Stage II (23), Stage III (68), Stage IV (12) and unknown stage (6).

The non-ovarian cancer sample set includes the following ovarian conditions: benign (104), normal ovary (29) and “low malignant potential/borderline (3). The sample set also includes serum from patients with other cancers: cervical cancer (7), endometrial cancer (6) and uterine cancer (1).

Antibodies that are specific for a biomarker antigen polypeptide of the invention are readily generated as monoclonal antibodies or as polyclonal antisera, or may be produced as genetically engineered immunoglobulins (Ig) that are designed to have desirable properties using methods well known in the art. For example, by way of illustration and not limitation, antibodies may include recombinant IgGs, chimeric fusion proteins having immunoglobulin derived sequences or “humanized” antibodies (see, e.g., U.S. Pat. Nos. 5,693,762; 5,585,089; 4,816,567; 5,225,539; 5,530,101; and references cited therein) that may all be used for detection of a human biomarker polypeptide according to the methods described herein. Such antibodies may be prepared as provided herein, including by immunization with biomarker polypeptides as described below. For example, as provided herein, nucleic acid sequences encoding biomarker polypeptides are disclosed, such that those skilled in the art may routinely prepare these polypeptides for use as immunogens.

The term “antibodies” includes polyclonal antibodies, monoclonal antibodies, fragments thereof such as F(ab′).sub.2, and Fab fragments, as well as any naturally occurring or recombinantly produced binding partners, which are molecules that specifically bind a biomarker polypeptide. Antibodies are defined to be “immunospecific” or specifically binding if they bind HE4a polypeptide with a K.sub.a of greater than or equal to about 10⁻⁴ M, preferably of greater than or equal to about 10⁻⁵M, more preferably of greater than or equal to about 10 and still more preferably of greater than or equal to about 10.sup . . . sup.7 M.sup.-1. Affinities of binding partners ⁻⁶ M or antibodies can be readily determined using conventional techniques, for example those described by Scatchard et al., Ann. N.Y. Acad. Sci. 51:660 (1949). Determination of other proteins as binding partners of a biomarker polypeptide can be performed using any of a number of known methods for identifying and obtaining proteins that specifically interact with other proteins or polypeptides, for example, a yeast two-hybrid screening system such as that described in U.S. Pat. No. 5,283,173 and U.S. Pat. No. 5,468,614, or the equivalent. The methods described herein also includes the use of a biomarker polypeptide, and peptides based on the amino acid sequence of a biomarker polypeptide, to prepare binding partners and antibodies that specifically bind to a biomarker polypeptide.

Antibodies may generally be prepared by any of a variety of techniques known to those of ordinary skill in the art (see, e.g., Harlow and Lane, Antibodies: A Laboratory Manual, Cold Spring Harbor Laboratory, 1988). In one such technique, an immunogen comprising a biomarker polypeptide, for example a cell having a biomarker polypeptide on its surface or an isolated biomarker polypeptide is initially injected into a suitable animal (e.g., mice, rats, rabbits, sheep and goats), preferably according to a predetermined schedule incorporating one or more booster immunizations, and the animals are bled periodically. Polyclonal antibodies specific for the biomarker polypeptide may then be purified from such antisera by, for example, affinity chromatography using the polypeptide coupled to a suitable solid support.

Monoclonal antibodies specific for biomarker polypeptides or variants thereof may be prepared, for example, using the technique of Kohler and Milstein (1976 Eur. J. Immunol 6.511-519), and improvements thereto. Briefly, these methods involve the preparation of immortal cell lines capable of producing antibodies having the desired specificity (i.e., reactivity with the mesothelin polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. For example, the spleen cells and myeloma cells may be combined with a membrane fusion promoting agent such as polyethylene glycol or a nonionic detergent for a few minutes, and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide. Hybridomas having high reactivity and specificity are preferred. Hybridomas that generate monoclonal antibodies that specifically bind to biomarker polypeptides are contemplated by the methods described herein.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse or other suitable host. Monoclonal antibodies may then be harvested from the ascites fluid or the blood. Contaminants may be removed from the antibodies by conventional techniques, such as chromatography, gel filtration, precipitation, and extraction. For example, antibodies may be purified by chromatography on immobilized Protein G or Protein A using standard techniques.

Within certain embodiments, the use of antigen-binding fragments of antibodies may be preferred. Such fragments include Fab fragments, which may be prepared using standard techniques (e.g., by digestion with papain to yield Fab and Fc fragments). The Fab and Fc fragments may be separated by affinity chromatography (e.g., on immobilized protein A columns), using standard techniques. Such techniques are well known in the art, see, e.g., Weir, D. M., Handbook of Experimental Immunology, 1986, Blackwell Scientific, Boston.

Multifunctional fusion proteins having specific binding affinities for pre-selected antigens by virtue of immunoglobulin V-region domains encoded by DNA sequences linked in-frame to sequences encoding various effector proteins are known in the art, for example, as disclosed in EP-B1-0318554, U.S. Pat. No. 5,132,405, U.S. Pat. No. 5,091,513 and U.S. Pat. No. 5,476,786. Such effector proteins include polypeptide domains that may be used to detect binding of the fusion protein by any of a variety of techniques with which those skilled in the art will be familiar, including but not limited to a biotin mimetic sequence (see, e.g., Luo et al., 1998 J. Biotechnol. 65:225 and references cited therein), direct covalent modification with a detectable labeling moiety, non-covalent binding to a specific labeled reporter molecule, enzymatic modification of a detectable substrate or immobilization (covalent or non-covalent) on a solid-phase support.

Single chain antibodies for use in the methods described herein may also be generated and selected by a method such as phage display (see, e.g., U.S. Pat. No. 5,223,409; Schlebusch et al., 1997 Hybridoma 16:47; and references cited therein). Briefly, in this method, DNA sequences are inserted into the gene III or gene VIII gene of a filamentous phage, such as M13. Several vectors with multicloning sites have been developed for insertion (McLafferty et al., Gene 128:29-36, 1993; Scott and Smith, Science 249:386-390, 1990; Smith and Scott, Methods Enzymol. 217:228-257, 1993). The inserted DNA sequences may be randomly generated or may be variants of a known binding domain for binding to a biomarker polypeptide. Single chain antibodies may readily be generated using this method. Generally, the inserts encode from 6 to 20 amino acids. The peptide encoded by the inserted sequence is displayed on the surface of the bacteriophage. Bacteriophage expressing a binding domain for a biomarker polypeptide are selected by binding to an immobilized biomarker polypeptide, for example a recombinant polypeptide prepared using methods well known in the art and nucleic acid coding sequences as disclosed herein. Unbound phage are removed by a wash, typically containing 10 mM Tris, 1 mM EDTA, and without salt or with a low salt concentration. Bound phage are eluted with a salt containing buffer, for example. The NaCl concentration is increased in a step-wise fashion until all the phage are eluted. Typically, phage binding with higher affinity will be released by higher salt concentrations. Eluted phage are propagated in the bacteria host. Further rounds of selection may be performed to select for a few phage binding with high affinity. The DNA sequence of the insert in the binding phage is then determined. Once the predicted amino acid sequence of the binding peptide is known, sufficient peptide for use herein as an antibody specific for a biomarker polypeptide may be made either by recombinant means or synthetically. Recombinant means are used when the antibody is produced as a fusion protein. The peptide may also be generated as a tandem array of two or more similar or dissimilar peptides, in order to maximize affinity or binding.

To detect an antigenic determinant reactive with an antibody specific for a biomarker polypeptide, the detection reagent is typically an antibody, which may be prepared as described herein. There are a variety of assay formats known to those of ordinary skill in the art for using an antibody to detect a polypeptide in a sample, including but not limited to enzyme linked immunosorbent assay (ELISA), radioimmunoassay (RIA), immunofluorimetry, immunoprecipitation, equilibrium dialysis, immunodiffusion and other techniques. See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston. For example, the assay may be performed in a Western blot format, wherein a protein preparation from the biological sample is submitted to gel electrophoresis, transferred to a suitable membrane and allowed to react with the antibody. The presence of the antibody on the membrane may then be detected using a suitable detection reagent, as is well known in the art and described below.

In another embodiment, the assay involves the use of an antibody immobilized on a solid support to bind to the target biomarker polypeptide and remove it from the remainder of the sample. The bound biomarker polypeptide may then be detected using a second antibody reactive with a distinct biomarker polypeptide antigenic determinant, for example, a reagent that contains a detectable reporter moiety. Alternatively, a competitive assay may be utilized, in which a biomarker polypeptide is labeled with a detectable reporter moiety and allowed to bind to the immobilized biomarker polypeptide specific antibody after incubation of the immobilized antibody with the sample. The extent to which components of the sample inhibit the binding of the labeled polypeptide to the antibody is indicative of the reactivity of the sample with the immobilized antibody, and as a result, indicative of the level of biomarker polypeptides in the sample.

The solid support may be any material known to those of ordinary skill in the art to which the antibody may be attached, such as a test well in a microtiter plate, a nitrocellulose filter or another suitable membrane. Alternatively, the support may be a bead or disc, such as glass, fiberglass, latex or a plastic such as polystyrene or polyvinylchloride. The antibody may be immobilized on the solid support using a variety of techniques known to those in the art, which are amply described in the patent and scientific literature.

In certain preferred embodiments, the assay for detection of biomarker antigen polypeptide in a sample is a two-antibody sandwich assay. This assay may be performed by first contacting a biomarker polypeptide-specific antibody that has been immobilized on a solid support, commonly the well of a microtiter plate, with the biological sample, such that a soluble molecule naturally occurring in the sample and having an antigenic determinant that is reactive with the antibody is allowed to bind to the immobilized antibody (e.g., a 30 minute incubation time at room temperature is generally sufficient) to form an antigen-antibody complex or an immune complex. Unbound constituents of the sample are then removed from the immobilized immune complexes. Next, a second antibody specific for a biomarker antigen polypeptide is added, wherein the antigen combining site of the second antibody does not competitively inhibit binding of the antigen combining site of the immobilized first antibody to a biomarker polypeptide. The second antibody may be detectably labeled as provided herein, such that it may be directly detected. Alternatively, the second antibody may be indirectly detected through the use of a detectably labeled secondary (or “second stage”) anti-antibody, or by using a specific detection reagent as provided herein. The methods described herein are not limited to any particular detection procedure, as those having familiarity with immunoassays will appreciate that there are numerous reagents and configurations for immunologically detecting a particular antigen (e.g., a mesothelin polypeptide) in a two-antibody sandwich immunoassay.

In certain preferred embodiments of the methods described herein using the two-antibody sandwich assay described above, the first, immobilized antibody specific for a bioma antigen polypeptide is a polyclonal antibody and the second antibody specific for a biomarker antigen polypeptide is a polyclonal antibody. Any combination of non-competitive biomarker antibodies could be used with the methods described herein. Including monoclonal antibodies, polyclonal antibodies and combinations thereof. In certain other embodiments of the methods described herein the first, immobilized antibody specific for a biomarker antigen polypeptide is a monoclonal antibody and the second antibody specific for a biomarker antigen polypeptide is a polyclonal antibody. In certain other embodiments of the methods described herein the first, immobilized antibody specific for a biomarker antigen polypeptide is a polyclonal antibody and the second antibody specific for a biomarker antigen polypeptide is a monoclonal antibody. In certain other highly preferred embodiments of the methods described herein the first, immobilized antibody specific for a biomarker antigen polypeptide is a monoclonal antibody and the second antibody specific for a biomarker antigen polypeptide is a monoclonal antibody. In other preferred embodiments of the methods described herein the first, immobilized antibody specific for a biomarker antigen polypeptide and/or the second antibody specific for a biomarker antigen polypeptide may be any of the kinds of antibodies known in the art and referred to herein, for example by way of illustration and not limitation, Fab fragments, F(ab′).sub.2 fragments, immunoglobulin V-region fusion proteins or single chain antibodies. Those familiar with the art will appreciate that the methods described herein encompass the use of other antibody forms, fragments, derivatives and the like in the methods disclosed and claimed herein.

In certain particularly preferred embodiments, the second antibody may contain a detectable reporter moiety or label such as an enzyme, dye, radionuclide, luminescent group, fluorescent group or biotin, or the like. Any reporter moiety or label could be used with the methods described herein, so long as the signal of such is directly related or proportional to the quantity of antibody remaining on the support after wash. The amount of the second antibody that remains bound to the solid support is then determined using a method appropriate for the specific detectable reporter moiety or label. For radioactive groups, scintillation counting or autoradiographic methods are generally appropriate. Antibody-enzyme conjugates may be prepared using a variety of coupling techniques (for review see, e.g., Scouten, W. H., Methods in Enzymology 135:30-65, 1987). Spectroscopic methods may be used to detect dyes (including, for example, colorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of antigen in a sample, using well known techniques.

In another embodiment, the methods described herein involve use of a biomarker antigen polypeptide as provided herein to screen for the presence of a malignant condition by detection of immunospecifically reactive antibodies in a biological sample from a biological source or subject. According to this embodiment, a biomarker antigen polypeptide (or a fragment or variant thereof including a truncated biomarker antigen polypeptide as provided herein) is detectably labeled and contacted with a biological sample to detect binding to the biomarker antigen polypeptide of an antibody naturally occurring in soluble form in the sample. For example, the biomarker antigen polypeptide may be labeled biosynthetically by using the sequences disclosed herein in concert with well known methods such as incorporation during in vitro translation of a readily detectable (e.g. radioactively labeled) amino acid, or by using other detectable reporter moieties such as those described above. Without wishing to be bound by theory, this embodiment of the methods described herein contemplates that certain biomarker polypeptides such as the biomarker fusion polypeptides disclosed herein, may provide peptides that are particularly immunogenic and so give rise to specific and detectable antibodies. For example, according to this theory certain biomarker fusion polypeptides may represent “non-self” antigens that provoke an avid immune response, while biomarker polypeptides that lack fusion domains may be viewed by the immune system as more resembling “self” antigens that do not readily elicit humoral or cell-mediated immunity.

Analyte levels in the samples discussed in this specification were measured using a high-throughput, multi-analyte immunoassay platform. A preferred platform is the Luminex® MAP system as developed by Rules-Based Medicine, Inc. in Austin, Tex. It is described on the company's website and also, for example, in publications such as Chandler et al., “Methods and kits for the diagnosis of acute coronary syndrome, U.S. Patent Application 2007/0003981, published Jan. 4, 2007, and a related application of Spain et al., “Universal Shotgun Assay,” U.S. Patent Application 2005/0221363, published Oct. 6, 2005. This platform has previously been described in Lokshin (2007) and generated data used in other analyses of ovarian cancer biomarkers. However, any immunoassay platform or system may be used.

In brief, to describe a preferred analyte measurement system, the MAP platform incorporates polystyrene microspheres that are dyed internally with two spectrally distinct fluorochromes. By using accurate ratios of the fluorochromes, an array is created consisting of 100 different microsphere sets with specific spectral addresses. Each microsphere set can display a different surface reactant. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex® analyzer. High-speed digital signal processing classifies the microsphere based on its spectral address and quantifies the reaction on the surface in a few seconds per sample.

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

An immunoassay is an assay that uses an antibody to specifically bind an antigen (e.g., a marker). The immunoassay is characterized by the use of specific binding properties of a particular antibody to isolate, target, and/or quantify the antigen. The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a marker from specific species such as rat, mouse, or human can be selected to obtain only those polyclonal antibodies that are specifically immunoreactive with that marker and not with other proteins, except for polymorphic variants and alleles of the marker. This selection may be achieved by subtracting out antibodies that cross-react with the marker molecules from other species.

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Typically a specific or selective reaction will be at least twice background signal or noise and more typically more than 10 to 100 times background.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of, or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asci, ed. 1993); Basic and Clinical Immunology (Stites & Terr, eds., 7th ed. 1991); and Harlow & Lane, supra.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

Another method of measuring the biomarkers includes the use of a combinatorial ligand library synthesized on beads as described in Ser. No. 11/495,842, filed Jul. 28, 2006 and entitled “Methods for Reducing the range in Concentrations of Analyte Species in a Sample”; hereby incorporated by reference in its

Skilled artisans will recognize that a wide variety of analytical techniques may be used to determine the levels of biomarkers in a sample as is described and claimed in this specification. Other types of binding reagents available to persons skilled in the art may be utilized to measure the levels of the indicated analytes in a sample. For example, a variety of binding agents or binding reagents appropriate to evaluate the levels of a given analyte may readily be identified in the scientific literature. Generally, an appropriate binding agent will bind specifically to an analyte, in other words, it reacts at a detectable level with the analyte but does not react detectably (or reacts with limited cross-reactivity) with other or unrelated analytes. It is contemplated that appropriate binding agents include polyclonal and monoclonal antibodies, aptamers, RNA molecules and the like. Spectrometric methods also may be used to measure the levels of analytes, including immunofluorescence, mass spectrometry, nuclear magnetic resonance and optical spectrometric methods. Depending on the binding agent to be utilized, the samples may be processed, for example, by dilution, purification, denaturation, digestion, fragmentation and the like before analysis as would be known to persons skilled in the art. Also, gene expression, for example, in a tumor cell or lymphocyte also may be determined.

It is also contemplated that the identified biomarkers may have multiple epitopes for immunassays and/or binding sites for other types of binding agents. Thus, it is contemplated that peptide fragments or other epitopes of the identified biomarkers, isoforms of specific proteins and even compounds upstream or downstream in a biological pathway or that have been post-translationally modified may be substituted for the identified analytes or biomarkers so long as the relevant and relative stoichiometries are taken into account appropriately. Skilled artisans will recognize that alternative antibodies and binding agents can be used to determine the levels of any particular analyte, so long as their various specificities and binding affinities are factored into the analysis.

A variety of algorithms may be used to measure or determine the levels of expression of the analytes or biomarkers used in the methods and test kits of the present invention. It is generally contemplated that such algorithms will be capable of measuring analyte levels beyond the measurement of simple cut-off values. Thus, it is contemplated that the results of such algorithms will generically be classified as multivariate index analyses by the U.S. Food and Drug Administration. Specific types of algorithms include: knowledge discovery engine (KDE™), regression analysis, discriminant analysis, classification tree analysis, random forests, ProteomeQuest®, support vector machine, One R, kNN and heuristic naive Bayes analysis, neural nets and variants thereof.

While there are many very sophisticated algorithms that calculate the probability of an unknown sample being a cancer, a simple logistic regression model typically works quite well for building a diagnostic model based on the measurement of a few markers (preferably less than about five). The theory behind that is well known to persons skilled in the art. There are also many options regarding which software can be use—both commercial and free (and open source) packages.

The training of a logistic model consists of separating the samples into cases and controls and then use the software chosen to optimize the regression coefficients, one for each marker, plus one bias parameter, so as to maximize the likelihood of the logistic model applied to the training data.

Once trained, the set of regression coefficients defines the logistic model. A person skilled in the art can easily use this type of diagnostic model to predict the probability of any new samples being identified as a case or control, by plugging the levels of the biomarkers into the logistic equation. Furthermore, an ROC can also be constructed by computing the sensitivities and specificities as the cutoff value of the computed probability varies from 0 to 1. The use of Logistic Regression to calculate a probability comprises the following steps: 1) Measure the levels of the biomarkers:

For each biomarker, its level is measured and recorded. As guidance to practitioners, the following discussion assumes the use of N biomarkers, and their designated measured levels are x₁, x₂, . . . xₙ—

2) Compute the ‘z’ value:

A central quantity to compute in a logistic regression model is the ‘z’ parameter. It is defined as follows,

z=β₀+β₁x₁+β₂x₂+ . . . +βₙxₙ eq (1)

The parameter β0 is called the bias or intercept, while β₁, β₂, . . . βₙ are called weights. Specifying the βs would define a logistic regression model. Typically, a training process determines the βs where samples with a known state of either “disease” or “benign” are used to optimize a likelihood function by varying the βs. Once the training process is completed, the values of the βs will be chosen to yield the optimal likelihood of a correct determination.

For an unknown sample with measured biomarker levels: x₁, x₂, . . . xₙ and a predetermined set of βs, a person skilled in the art can compute the value of z according eq (1).

3) Compute the value of the Logistic Function:

The Logistic Function, f(z), is defined as

f(z)=e^{z}/(e^{z}+1) eq (2)

Given the value of z computed in 2), one can evaluate f(z) according to eq (2). In some applications, the natural logarithm of z is used instead of just z in eq (2).

4) Make A Diagnostic Call:

The Logistic Function yields a value between (0.0 and 1.0), for any value of z. In a typical application, a cutoff of 0.5 is used to differentiate between the controls and the cases. Thus a sample with a score that is >0.5 would be called a case, while a sample with a score that is <=0.5 would be called a control. In some applications of the diagnostic process, other cutoff values (for example, 0.65) are used.

### EXAMPLES

### Example 1

The following discussion and examples are provided to describe and illustrate the present invention. As such, they should not be construed to limit the scope of the invention. Those skilled in the art will well appreciate that many other embodiments also fall within the scope of the invention, as it is described in this specification and the claims.

### Analysis of Data Using the Knowledge Discovery Engine.

Correlogic has described the use of evolutionary and pattern recognition algorithms in evaluating complex data sets, including the Knowledge Discovery Engine (KDE™) and ProteomeQuest®. See, for example, Hitt et al., U.S. Pat. No. 6,925,389, “Process for Discriminating Between Biological States Based on Hidden Patterns From Biological Data” (issued Aug. 2, 2005); Hitt, U.S. Pat. No. 7,096,206, “Heuristic Method of Classification,” (issued Aug. 22, 2006) and Hitt, U.S. Pat. No. 7,240,038, “Heuristic Method of Classification,” (to be issued Jul. 3, 2007). The use of this technology to evaluate mass spectral data derived from ovarian cancer samples is further elucidated in Hitt et al., “Multiple high-resolution serum proteomic features for ovarian cancer detection,” U.S. Published Patent Application 2006/0064253, published Mar. 23, 2006.

When analyzing the data set by Correlogic's Knowledge Discovery Engine, the following five-biomarker panels were found to provide sensitivities and specificities for various stages of ovarian cancer as set forth in Table I. Specifically, KDE Model 1 [2_{—}0008_{—}20] returned a relatively high accuracy for Stage I ovarian cancer and included these markers: Cancer Antigen 19-9 (CA19-9, Swiss-Prot Accession Number: Q9BXJ9), C Reactive Protein (CRP, Swiss-Prot Accession Number: P02741), Fibroblast Growth Factor-basic Protein (FGF-basic, Swiss-Prot Accession Number: P09038) and Myoglobin (Swiss-Prot Accession Number: P02144). KDE Model 2 [4_{—}0002-10] returned a relatively high accuracy for Stage III, IV and “advanced” ovarian cancer and included these markers: Hepatitis C NS4 Antibody (Hep C NS4 Ab), Ribosomal P Antibody and CRP. KDE Model 3 [4_{—}0009_{—}140] returned a relatively high accuracy for Stage I and included these markers: CA 19-9, TGF alpha, EN-RAGE (Swiss-Prot Accession Number: P80511), Epidermal Growth Factor (EGF, Swiss-Prot Accession Number: P01133) and HSP 90 alpha antibody. KDE Model 4 [4_{—}0026_{—}100] returned a relatively high accuracy for Stage II and Stages III, W and “advanced” ovarian cancers and included these markers: EN-RAGE, EGF, Cancer Antigen 125 (CA125, Swiss-Prot Accession Number: Q14596), Fibrinogen (Swiss-Prot Accession Number: Alpha chain P02671; Beta chain P02675; Gamma chain P02679), Apolipoprotein CHI (ApoCIII, Swiss-Prot Accession Number: P02656), Cholera Toxin and CA 19-9. KDE Model 5 [4_{—}0027_{—}20] also returned a relatively high accuracy for Stage II and Stages III, IV and “advanced” ovarian cancers and included these markers: Proteinase 3 (cANCA) antibody, Fibrinogen, CA 125, EGF, CD40 (Swiss-Prot Accession Number: Q6P2H9), Thyroid Stimulating Hormone (TSH, Swiss-Prot Accession Number: Alpha P01215; Beta P01222 P02679, Leptin (Swiss-Prot Accession Number: P41159), CA 19-9 and Lymphotactin (Swiss-Prot Accession Number: P47992). It is contemplated that skilled artisans could use the KDE analytical tools to identify other, potentially useful sets of biomarkers for predictive or diagnostic value based on the levels of selected analytes. Note that the KDE algorithm may select and utilize various markers based on their relative abundances; and that a given marker, for example the level of cholera toxin in Model W may be zero but is relevant in combination with the other markers selected in a particular grouping.

Skilled artisans will recognize that a limited size data set as was used in this specification may lead to different results, for example, different panels of markers and varying accuracies when comparing the relative performance of KDE with other analytical techniques. These particular KDE models were built on a relatively small data set using 40 stage I ovarian cancers and 40 normal/benigns and were tested blindly on the balance of the stage II, III/IV described above. Thus, the specificity is of the stage I samples reflects sample set size and potential overfitting. The drop in specificity for the balance of the non-ovarian cancer samples also is expected given the relatively larger size of the testing set relative to the training set. Overall, the biomarker panel developed for the stage I samples also provides potentially useful predictive and diagnostic assays for later stages of ovarian cancer given the high sensitivity values.

However, these examples of biomarker panels illustrate that there are a number of parameters that can be adjusted to impact model performance. For instance in these cases a variety of different numbers of features are combined together, a variety of match values are used, a variety of different lengths of evolution of the genetic algorithm are used and models differing in the number of nodes are generated. By routine experimentation apparent to one skilled in the art, combinations of these parameters can be used to generate other models of clinically relevant performance.

TABLE I
Results of Analysis Using Knowledge Discovery Engine to develop a stage I specific classification model.

| | | | | | Sensitivity | Specificity | Accuracy | Sensitivity | Sensitivity | |
|---|---|---|---|---|---|---|---|---|---|---|
| Model Name | Feature | Match | Generation | Node | Stage I | Stage I | Stage I | Stage II | Stage III-IV | Specificity |
| 2_0008_20 | 4 | 0.9 | 20 | 12 | 75 | 100 | 87.5 | 60.9 | 46.5 | 82.6 |
| 4_0002_10 | 3 | 0.7 | 10 | 4 | 75 | 100 | 87.5 | 69.6 | 82.6 | 56 |
| 4_0009_140 | 5 | 0.6 | 140 | 5 | 75 | 100 | 87.5 | 43.5 | 39.5 | 71.6 |
| 4_0026_100 | 9 | 0.7 | 100 | 5 | 87.5 | 100 | 93.8 | 78.3 | 84.9 | 67 |
| 4_0027_20 | 9 | 0.8 | 20 | 5 | 87.5 | 100 | 93.8 | 78.3 | 84.9 | 60.6 |

Methods and Analysis Using Random Forests.

A preferred analytical technique, known to skilled artisans, is that of Breiman, Random Forests. Machine Learning, 2001. 45:5-32; as further described by Segel, Machine Learning Benchmarks and Random Forest Regression, 2004; and Robnik-Sikonja, Improving Random Forests, in Machine Learning, ECML, 2004 Proceedings, J. F. B. e. al., Editor, 2004, Springer: Berlin. Other variants of Random Forests are also useful and contemplated for the methods of the present invention, for example, Regression Forests, Survival Forests, and weighted population Random Forests.

Since each of the analyte assays is an independent measurement of a variable, under some circumstances, known to those skilled in the art, it is appropriate to scale the data to adjust for the differing variances of each assay. In such cases, biweight, MAD or equivalent scaling would be appropriate, although in some cases, scaling would not be expected to have a significant impact. A bootstrap layer on top of the Random Forests was used in obtaining the results discussed below.

In preferred embodiments of the present invention, contemplated panels of biomarkers are:

a. Cancer Antigen 125 (CA125, Swiss-Prot Accession Number: Q14596) and Epidermal Growth Factor Receptor (EGF-R, Swiss-Prot Accession Number: P00533).

b. CA125 and C Reactive Protein (CRP, Swiss-Prot Accession Number: P02741).

c. CA125, CRP and EGF-R.

d. Any one or more of CA125, CRP and EGF-R, plus any one or more of Ferritin (Swiss-Prot Accession Number: Heavy chain P02794; Light chain P02792), Interleukin-8 (IL-8, Swiss-Prot Accession Number: P10145), and Tissue Inhibitor of Metalloproteinases 1 (TIMP-1, Swiss-Prot Accession Number: P01033),

e. Any one of the biomarker panels presented in Table H and Table III.

f. Any of the foregoing panels of biomarkers (a e) plus any one or more of the other biomarkers in the following list if not previously included in the foregoing panels (a-e): Alpha-2 Macroglobulin (A2M, Swiss-Prot Accession Number: P01023), Apolipoprotein A1-1 (ApoA1, Swiss-Prot Accession Number: P02647), Apolipoprotein C-III (ApoCIII, Swiss-Prot Accession Number: P02656), Apolipoprotein H (ApoH, Swiss-Prot Accession Number: P02749), Beta-2 Microglobulin (B2M, Swiss-Prot Accession Number: P23560), Betacellulin (Swiss-Prot Accession Number: P35070), C Reactive Protein (CRP, Swiss-Prot Accession Number: P02741). Cancer Antigen 19-9 (CA19-9, Swiss-Prot Accession Number: Q9BXJ9), Cancer Antigen 125 (CA125, Swiss-Prot Accession Number: Q14596), Collagen Type 2 Antibody, Creatine Kinase-MB (CK-MB, Swiss-Prot Accession Number: Brain P12277; Muscle P06732), C Reactive Protein (CRP, Swiss-Prot Accession Number: P02741), Connective Tissue Growth Factor (CTGF, Swiss-Prot Accession Number: P29279), Double Stranded DNA Antibody (dsDNA Ab), EN-RAGE (Swiss-Prot Accession Number: P80511), Eotaxin (C-C motif chemokine 11, small-inducible cytokine A11 and Eosinophil chemotactic protein, Swiss-Prot Accession Number: P51671), Epidermal Growth Factor Receptor (EGF-R, Swiss-Prot Accession Number: P00533), Ferritin (Swiss-Prot Accession Number: Heavy chain P02794; Light chain P02792), Follicle-stimulating hormone (MI, Follicle-stimulating hormone beta subunit, FSH-beta, FSH-B, Follitropin beta chain, Follitropin subunit beta, Swiss-Prot Accession Number: P01225), Haptoglobin (Swiss-Prot Accession Number: P00738), flE4 (Major epididymis-specific protein E4, Epididymal secretory protein E4, Putative protease inhibitor WAP5 and WAP four-disulfide core domain protein 2, Swiss-Prot Accession Number: Q14508), Insulin (Swiss-Prot Accession Number: P01308), Insulin-like Growth Factor 1 (IGF-1, Swiss-Prot Accession Number: P01343), Insulin like growth factor II (IGF-II, Somatomedin-A, Swiss-Prot Accession Number: P01344), Insulin Factor VII (Swiss-Prot Accession Number: P08709), Interleukin-6 (IL-6, Swiss-Prot Accession Number: P05231), Interleukin-8 (IL-8, Swiss-Prot Accession Number: P10145), Interleukin-10 (IL-10, Swiss-Prot Accession Number: P22301), Interleukin-18 (IL-18, Swiss-Prot Accession Number: Q14116), Leptin (Swiss-Prot Accession Number: P41159), Lymphotactin (Swiss-Prot Accession Number: P47992), Macrophage-derived Chemokine (MDC, Swiss-Prot Accession Number: 000626), Macrophage Inhibotory Factor (SWISS PROT), Macrophage Inflammatory Protein 1 alpha (MIP-lalpha, Swiss-Prot Accession Number: P10147), Macrophage migration inhibitory factor (MIF, Phenylpyruvate tautomerase, Glycosylation-inhibiting factor, GIF, Swiss-Prot Accession Number: P14174), Myoglobin (Swiss-Prot Accession Number: P02144), Ostopontin (Bone sialoprotein 1, Secreted phosphoprotein 1, SPP-1, Urinary stone protein, Nephropontin, Uropontin, Swiss-Prot Accession Number: P10451), Pancreatic Islet Cells (GAD) Antibody, Prolactin (Swiss-Prot Accession Number: P01236), Stem Cell Factor (SCF, Swiss-Prot Accession Number: P21583), Tenascin C (Swiss-Prot Accession Number: P24821), Tissue Inhibitor of Metalloproteinases 1 (TIMP-1, Swiss-Prot Accession Number: P01033), Tumor Necrosis Factor-alpha (TNF-alpha, Swiss-Prot Accession Number: P01375), Tumor Necrosis Factor RII (TNF-RII, Swiss-Prot Accession Number: Q92956), von Willebrand Factor (vWF, Swiss-Prot Accession Number: PO4275) and the other biomarkers identified as being informative for cancer in the references cited in this specification.

Using the Random Forests analytical approach, a preferred seven biomarker panel was identified that has a high predictive value for Stage I ovarian cancer. It includes: ApoA1, ApoCIII, CA125, CRP, EGF-R, IL-18 and Tenascin. In the course of building and selecting the relatively more accurate models for Stage I cancers generated by Random Forests using these biomarkers, the sensitivity for Stage I ovarian cancers ranged from about 80% to about 85%. Sensitivity was also about 95 for Stage II and about 94% sensitive for Stage III/IV. The overall specificity was about 70%.

Similarly, a preferred seven biomarker panel was identified that has a high predictive value for Stage H. It includes: B2M, CA125, CK-MB, CRP, Ferritin, IL-8 and TIMP1. A preferred model for Stage H had a sensitivity of about 82% and a specificity of about 88%.

For Stage III, Stage IV and Advanced ovarian cancer, the following 19 biomarker panel was identified: A2M, CA125, CRP, CTGF, EGF-R, EN-RAGE, Ferritin, Haptoglobin, IGF-1, IL-8, IL-10, Insulin, Leptin, Lymphotactin, MDC, TIMP-1, TNF-alpha, TNF-RII, vWF. A preferred model for Stage III/IV had a sensitivity of about 86% and a specificity of about 89%.

Other preferred biomarker or analyte panels for detecting, diagnosing and monitoring ovarian cancer are shown in Table H and in Table TIT. These panels include CA-125, CRP and EGF-R and, in most cases, CA19-9. In Table II, 20 such panels of seven analytes each selected from 20 preferred analytes are displayed in columns numbered 1 through 20. In Table III, another 20 such panels of seven analytes each selected from 23 preferred analytes are displayed in columns numbered 1 through 20.

TABLE II
Additional Biomarker Panels

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| CA125 | x | x | x | x | x | x | x | x | x | x | x | x | x | x | x | x | x | x | x | x |
| CRP | x | x | x | x | x | x | x | x | x | x | x | x | x | x | x | x | x | x | x | x |
| EGF-R | x | x | x | x | x | x | x | x | x | x | x | x | x | x | x | x | x | x | x | x |
| CA19-9 | x | x | x | x | x | x | x | x | x | | x | x | x | x | x | x | x | x | x | x |
| Haptoglobin | | | | | | | | | | | | | | | | | | | | |
| Serum Amyloid P | | | x | | | x | | | x | | | | | | | | | | | |
| Apo A1 | | | | | | | x | | x | | | | | | | | | | | |
| IL-6 | | | x | x | | | | x | | x | | | | | | | | x | | x |
| Myoglobin | | | | | x | | | | | x | x | x | | x | x | x | x | x | x | x |
| MIP-1a | x | x | x | x | | | x | x | x | x | x | x | x | | | x | | | | |
| EN-RAGE | | | | | | | | | | | | | | | | | | | | |
| CK-MB | | | | | | | | | | | | | | | | | | | | |
| vWF | | x | | x | | | x | | | | | | | | | | | | | |
| Leptin | | | | | | | | | | | | | | | | x | x | | | |
| Apo CIII | | | | | x | x | | | | | | | | | | | | | x | |
| Growth Hormone | | | | | | | | | | | x | | x | x | x | | x | x | | |
| IL-10 | | | | | | | | | | | | | | | | | | | | |
| IL-18 | x | | | | x | x | | x | | | | x | | x | | | | | x | x |
| Myeloperoxidase | | | | | | | | | | x | | | x | | | | | | | |
| VCAM-1 | x | x | | | | | | | | | | | | | x | | | | | |

TABLE III
Additional Biomarker Panels

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| CA125 | x | x | x | x | x | x | x | x | x | x | x | x | x | x | x | x | x | x | x | x |
| CRP | x | x | x | x | x | x | x | x | x | x | x | x | x | x | x | x | x | x | x | x |
| EGF-R | x | x | x | x | x | x | x | x | x | x | x | x | x | x | x | x | x | x | x | x |
| CA19-9 | x | x | x | x | x | x | x | x | x | x | x | | x | x | x | x | x | x | x | x |
| Haptoglobin | | | | | | | | | | | | | | | | | | | | |
| Serum Amyloid P | | | | | | | x | | | | | x | | | | | x | | | |
| Apo A1 | | | x | | | | | | | x | | | | | | | | | | |
| IL-6 | | | | | | | | | | | x | x | | x | x | | | x | x | |
| Myoglobin | x | x | x | x | x | x | x | x | x | x | | | | | | | | | | |
| MIP-1a | x | | x | x | x | x | x | x | x | | | x | | x | x | x | | x | | x |
| EN-RAGE | | | | | | | | | | | | | | | | | | | | |
| CK-MB | | | | | | | | | | | | | | | | | | | x | |
| vWF | | | | | | | | | | | x | | x | | | x | | | x | |
| Leptin | x | x | | | | | | | | x | | | | | | | | | | |
| Apo CIII | | | | | | | | x | | | x | | x | | | x | x | x | | |
| Growth Hormone | | | | | | | | | | | | | | | | | | | | |
| IL-10 | | | | | | | | | | | | | | | x | | | | | x |
| IL-18 | | | | | | | | | | | | | | | | | | | | |
| Myeloperoxidase | | x | | | | x | | | | | | x | | | | | | | | |
| VCAM-1 | | | | | | | | | | | | | | | | | | | | |
| Insulin | | | | x | | | | | | | | | | | | | | | | |
| Ferritin | | | | | x | | | | | | | | x | x | | | x | | | x |
| Haptoglobin | | | | | | | | | x | | | | | | | | | | | |

Other preferred biomarker panels (or models) for all stages of ovarian cancer include: (a) CA-125, CRP, EGF-R, CA-19-9, Apo-AI, Apo-CIII, IL-6, IL-18, MIP-1a, Tenascin C and Myoglobin; (b) CA125, CRP, CA19-9, EGF-R, Myoglobin, IL-18, Apo CIII; and (c) CA125, CRP, EGF-R, CA19-9, Apo CM, MIP-1a, Myoglobin, IL-18, IL-6, Apo AI, Tenascin C, vWF, Haptoglobin, IL-10. Optionally, any one or more of the following biomarkers may be added to these or to any of the other biomarker panels disclosed above in text or tables (to the extent that any such panels are not already specifically identified therein): vWF, Haptoglobin, IL-10, IGF-I, IGF-II, Prolactin, HE4, ACE, ASP and Resistin.

It is contemplated by the present inventors that additional, informative sets of analytes (or biomarkers) include any one or more, two or more, three or more and for or more of the analytes presented below in Table IV, as well as any of the biomarker sets in Tables I, II or III combined with any one or more of the analytes in Table IV, and any one or more of the markers in Table IV combined with any of the other biomarker sets discussed in Paragraphs 70-75, above, or identified elsewhere in this specification. Additional set of informative analytes for use in the test kits and methods of the present invention include any one or more of CA-125, CRP, ECG-R and HE-4 together with any one or more of the biomarkers in Table IV.

Thus, contemplated sets of biomarkers include combinations such as: CA-125, CRP and one or more (or two or more) of the biomarkers in Table IV; CA-125, EGF-R and any one or more (or two or more) of the biomarkers in Table IV; CA-125, HE-4 and any one or more (or two or more) of the biomarkers in Table IV; CRP, EGF-R and any one or more (or two or more) of the biomarkers in Table IV; CRP, HE-4 and any one or more (or two or more) of the biomarkers in Table IV; and EGF-R, HE-4 and any one or more (or two or more) of the biomarkers in Table IV. It is contemplated that markers of informative value in the foregoing biomarker sets according to the present invention include VCAM-1, IL-6R, IL-18R and sortillin.

Additionally, biomarker panels comprising any one or more (or two or more) of the biomarkers in Table IV together with any two or more, three or more and four or more of these three sets of biomarkers: (a) CA125, Transthyretin, ApoA-I, B2-microglobulin and Transferrin; (b) CA125 and leptin, prolactin, osteopontin, and insulin-like growth factor-II; and (c) OvaPlex: CA125, C-reactive protein, serum amyloid A, IL-6 and IL-8.

In general, soluble forms of these analytes are contemplated, including protein and peptide fragments and domains that are shed into the circulating blood and lymph streams. These analytes may be detected and analyzed in blood, lymph, serum, urine and other bodily fluids. Also contemplated in the compositions and methods of the present invention are autoantibodies against any of the disclosed biomarkers, as well as nucleotides that encode these biomarkers, and that may be detected and quantified as another indirect way to assess the levels of these markers. Aptamers and other compounds useful for the detection of such molecular species are well known to persons skilled in the art.

TABLE IV

| Analyte # | Informative Analytes |
|---|---|
| 1 | CA 15-3 (MUC-1) |
| 2 | Her2/Neu (erbB-2) |
| 3 | Kallikrein-5 |
| 4 | Macrophage Inhibitory Factor (MIF) |
| 5 | Osteopontin |
| 6 | TAG-72 |
| 7 | Total IGF-II |
| 8 | HE4 |
| 9 | Il6-R |
| 10 | Il6-R shedded form of full-length IL6-R |
| 11 | IL18-R |
| 12 | IL-18BP |
| 13 | VCAM-1 |
| 14 | IP-10 (interferon-gamma inducible 10 kD protein) |
| 15 | SMRP |
| 16 | TgII (tissue transglutaminase) |
| 17 | Exotaxin-1 |
| 18 | Cyfra 21-1(cytokeratin 19 fragment) |
| 19 | IGF2BP3 |
| 20 | TIMP-1 |
| 21 | Alpha-1 Antitrypsin |
| 22 | MMP7 |
| 23 | TAG-72 |
| 24 | IL-8 |
| 25 | IL-6 |
| 26 | Sortillin |
| 27 | CD40 |
| 28 | CA 15-3 |
| 29 | Alpha 1-Antichymotrypsin |
| 30 | VEGF |
| 21 | TTR (pre-albumin) |
| 22 | Haptoglobin |

Any two or more of the preferred biomarkers described above will have predictive value, however, adding one or more of the other preferred markers to any of the analytical panels described herein may increase the panel's predictive value for clinical purposes. For example, adding one or more of the different biomarkers listed above or otherwise identified in the references cited in this specification may also increase the biomarker panel's predictive value and are therefore expressly contemplated. Skilled artisans can readily assess the utility of such additional biomarkers. It is contemplated that additional biomarker appropriate for addition to the sets (or panels) of biomarkers disclosed or claimed in this specification will not result in a decrease in either sensitivity or specificity without a corresponding increase in either sensitivity or specificity or without a corresponding increase in robustness of the biomarker panel overall. A sensitivity and/or specificity of at least about 80% or higher are preferred, more preferably at least about 85% or higher, and most preferably at least about 90% or 95% or higher.

The results of the disclosed diagnostic may be output for the benefit of the user or diagnostician, or may otherwise be displayed on a medium such as, but not limited to, a computer screen, a computer readable medium, a piece of paper, or any other visible medium.

The foregoing embodiments and advantages of this invention are set forth, in part, in the preceding description and examples and, in part, will be apparent to persons skilled in the art from this description and examples and may be further realized from practicing the invention as disclosed herein. For example, the techniques of the present invention are readily applicable to monitoring the progression of ovarian cancer in an individual, by evaluating a specimen or biological sample as described above and then repeating the evaluation at one or more later points in time, such that a difference in the expression or disregulation of the relevant biomarkers over time is indicative of the progression of the ovarian cancer in that individual or the responsiveness to therapy. All references, patents, journal articles, web pages and other documents identified in this patent application are hereby incorporated by reference in their entireties.

### Example 2

Sera were from a prospective, collection undertaken specifically to develop and validate the performance of an ovarian cancer test. All samples were collected under a uniform protocol from 11 different sites, which were monitored for adherence. The Western Institutional Review Board (Olympia, Wash.) and the IRBs of the individual sites approved the studies under FDA Investigational Device Exemption (IDE) number G050132. The collection sites (and IRBs) were Cedars-Sinai Medical Center, Los Angeles, Calif. (Cedars-Sinai Institutional Review Board); Florida Gynecologic Oncology, Fort Meyers, FL (Lee Memorial Health System Institutional Review Committee); Florida Hospital Cancer institute, Orlando, Fla. (Florida Hospital Institutional Review Board); The Harry and Jeanette Weinberg Lancer Institute at Franklin Square Hospital, Baltimore, NM (MedStar Research Institute Georgetown Oncology Institutional Review Board); Holy Cross Hospital, Silver Spring, Md. (Holy Cross Institutional Review Board); North Shore—Long Island Jewish Health System, Manhasset, N.Y. (Institutional Review Board North Shore-Long Island Jewish Health System); SUNY at Stony Brook, N.Y., Stony Brook, N.Y. (Committee on Research Involving Human Subjects SUNY Stony Brook); University of Alabama at Birmingham, Birmingham, Ala. (The University of Alabama at Birmingham Institutional Review Board for Human Use); University of Southern California, Norris Cancer Center, Los Angeles, Calif. and Women's and Children's Hospital, Los Angeles, Calif. (University of Southern California Health Sciences Campus institutional. Review Board); Wake Forest University Health Sciences, Winston-Salem, N.C. (Institutional Review Board Wake Forest University School of Medicine); and Women and Infants Hospital of Rhode Island, Providence, R.I. (Institutional Review Board Women and Infants' Hospital of Rhode Island). The study inclusion criteria were women, at least 18 years of age, symptomatic of ovarian cancer according to the National Comprehensive Cancer Network (NCCN) Ovarian Cancer Treatment Guidelines for Patients, which includes women with or without a pelvic mass. Participants had to be scheduled for gynecologic surgery based on concern they had ovarian cancer, and post-surgical pathological evaluation of the ovaries and excised tissues was required to establish clinical truth of disease status. Exclusion criteria were women who did not meet the inclusion criteria, could not provide informed consent, were pregnant, or previously treated for ovarian cancer. Written informed consent was obtained for each participant in the study. All data were de-identified and no results were returned to the physicians or patients.

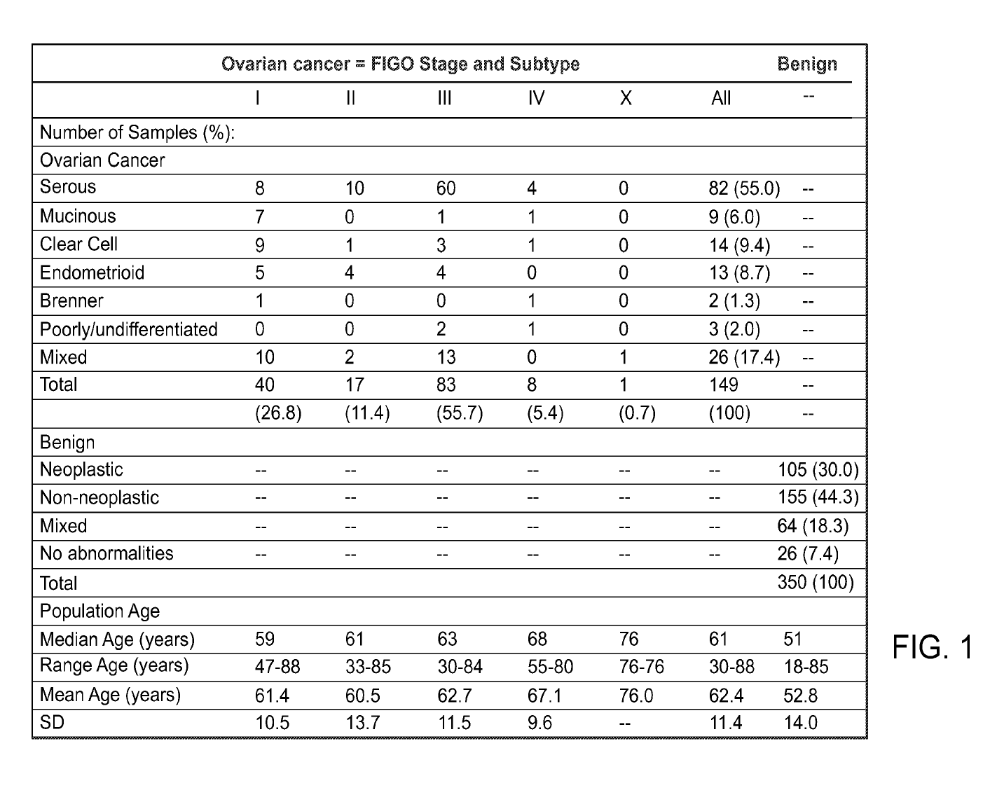

149 samples were used from the patients with pathology-confirmed ovarian cancer and 350 samples from the patients with pathology-confirmed benign conditions (FIG. 1). The ovarian cancer samples included all stages and common subtypes of the disease. The benign samples included the common types of benign conditions seen in the entire study population. Complete clinicopatbology reports, obtained following surgery, along with the patient age, race, staging, subtype and coded collection site accompanied each sample.

Prior to any intervention, blood samples (10 ml) were collected into red top glass Vacutainer tubes. The blood was clotted for at least 30 minutes at room temperature, centrifuged at 3,500 g for 10 minutes, and the resulting serum removed into pre-labeled cryotubes, and stored promptly at −80° C. Processing from blood draw to freezing was completed within 2 hours. All samples were shipped on dry ice to a single designated site for storage. To aliquot, all samples were thawed in a water and ice slurry then transferred into sample tubes labeled with coded identifiers that blinded all subsequent experimenters to the sample disease status.

### Multiplex Immunoassays

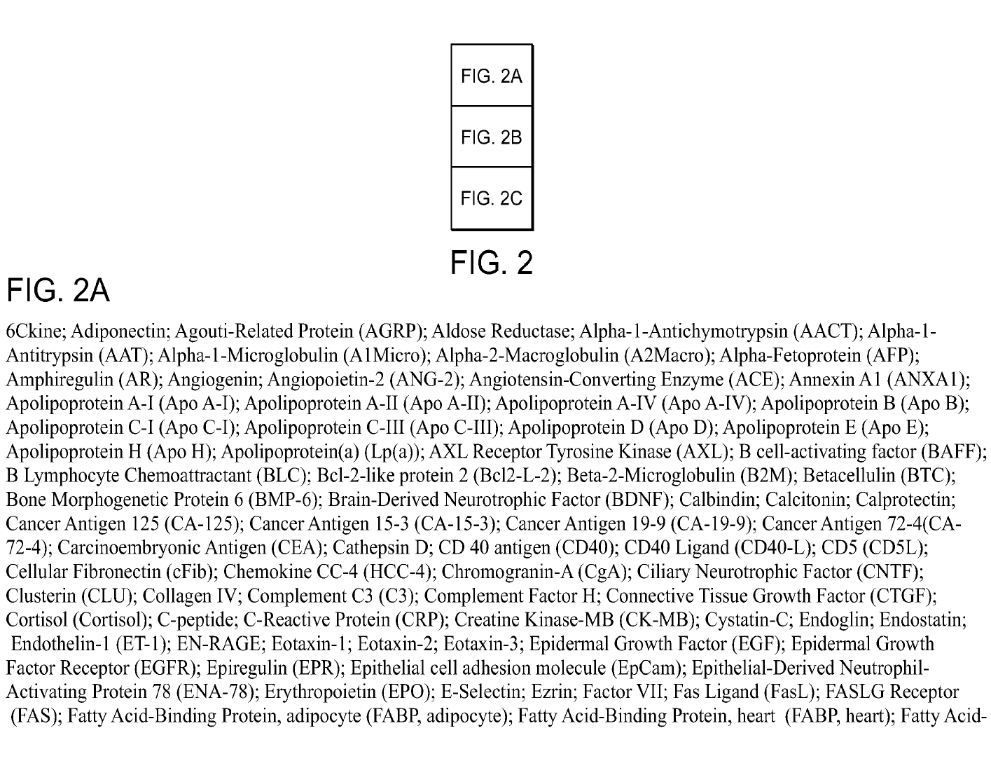

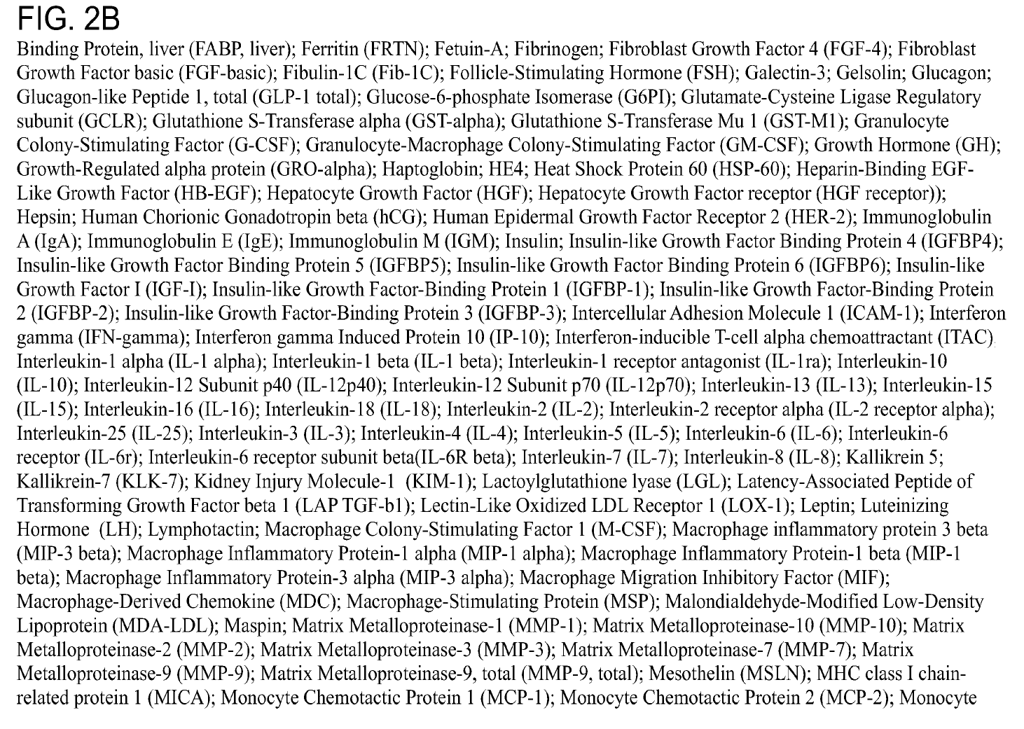

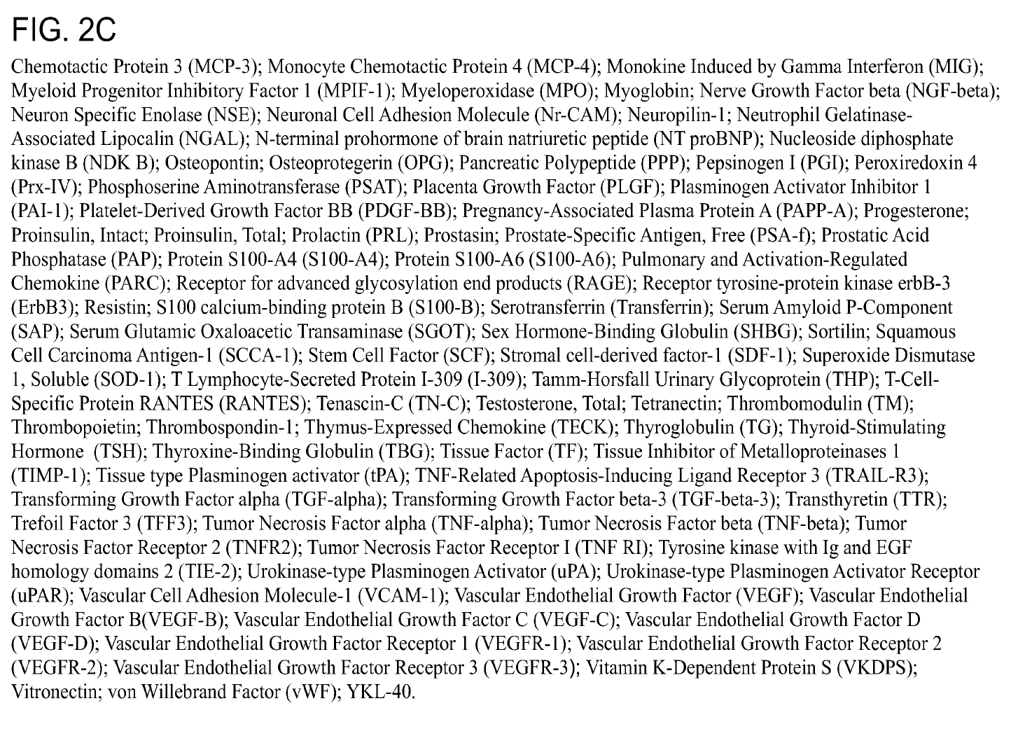

Two hundred and fifty nine serum biomarkers were measured using a set of proprietary multiplexed immunoassays (Human DiscoveryMAP® v1.0 and Human OncologyMAP® v1.0; (FIG. 2). Each assay was calibrated using an 8-point standard curve, performed in duplicate. Median Fluorescence Intensity (MFI) measurements were interpolated into final protein concentrations using curve-fitting software. Assay performance was verified using quality control (QC) samples at low, medium and high levels for each analyte in duplicate. All standard and QC samples were in a complex serum-based matrix to match the sample background matrix. Since sera were analyzed at a previously optimized dilution, any reading above the maximum concentration of the calibration curve was assigned the concentration of the highest standard, whereas any below the minimum concentration was assigned the value 0. For analysis, the sample run order was randomized to avoid any sequential bias due to presence or absence of disease, subtype or stage of disease, patient age, or age of serum sample.

### Data Analysis

Descriptive statistics, Receiver Operating Characteristic (ROC) curves and graphical displays (dot plots) for serum analyte concentrations were performed using commercially available software packages. Statistical differences were determined using the nonparametric Kruskal-Wallis test (ANOVA) followed by Dunn's multiple comparison post-test. For all statistical comparisons a P-value<0.05 was interpreted as statistically significant. A Pearson correlation matrix was created using a multi-spectral analysis application.

Using multiplexed immunoassays, the levels of 259 molecules were simultaneously measured in sera from 149 patients with pathology-confirmed epithelial ovarian cancer and 350 individuals with benign ovarian conditions (FIG. 1). To facilitate the determination of the ability of biomarkers to differentiate between symptomatically similar cancer and benign gynecological conditions, all samples were obtained from the same clinical population—women presenting for surgery primarily based on the presence of an adnexal mass. All samples were collected before any intervention and before the disease status was known. Disease status was subsequently identified by pathology exams of the excised tissue. Sera were collected using a single sample collection protocol that was monitored for compliance. The study was conducted prospectively at 11 sites that were also monitored for protocol adherence. This assured sample quality and removed the possibility of any collection, processing or biological biases in the sample set, a concern for many other studies. No normal healthy samples were used in this study, as they are typically easier to classify than benign conditions and introduce confounding factors such as lower stress levels compared to patients facing surgery. As expected, the median patient age was higher in individuals with ovarian cancer (61 years) than those with benign conditions (51 years) and increased with the stage of disease present (FIG. 1). The distribution of the ovarian cancer subtypes was similar to the distribution seen for all ovarian cancer cases in the US population as a whole, with a larger proportion of serous carcinoma (55%) than other subtypes (FIG. 1). The benign controls in the study were representative of common benign ovarian conditions including cystadenoma, cystadenofibroma and fibroma.

To ensure consistency and aid in biomarker comparisons, all 259 markers and 499 samples were measured on a single platform at a single site using a panel of rigorously qualified, high-throughput, multiplexed immunoassays. This survey built on our previous profiling of 104 serum biomarkers. The majority of the additional 155 serum biomarkers in the present study were developed as part of two NCI-funded Small Business Innovative Research (SBIR) awards specifically targeted at markers that had reasonable literature support to suggest a significant role in cancer biomarker. The selected biomarkers covered a broad range of biological functions, primarily implicated in cancer including cancer antigens, hormones, clotting factors, tissue modeling factors, lipoprotein constituents, proteases and protease inhibitors, markers of cardiovascular risk, growth factors, cytokine/chemokines, soluble forms of cell-signaling receptors, and inflammatory and acute phase reactants (FIG. 2). The present study is the broadest and most consistent single study of immunoassay profiling of molecules using fully characterized, quality-controlled samples.

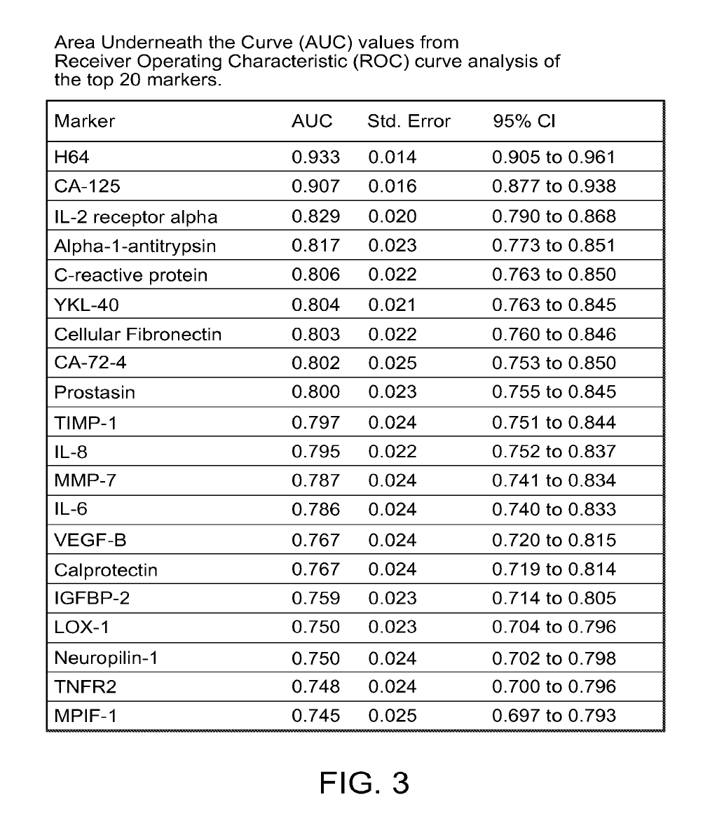

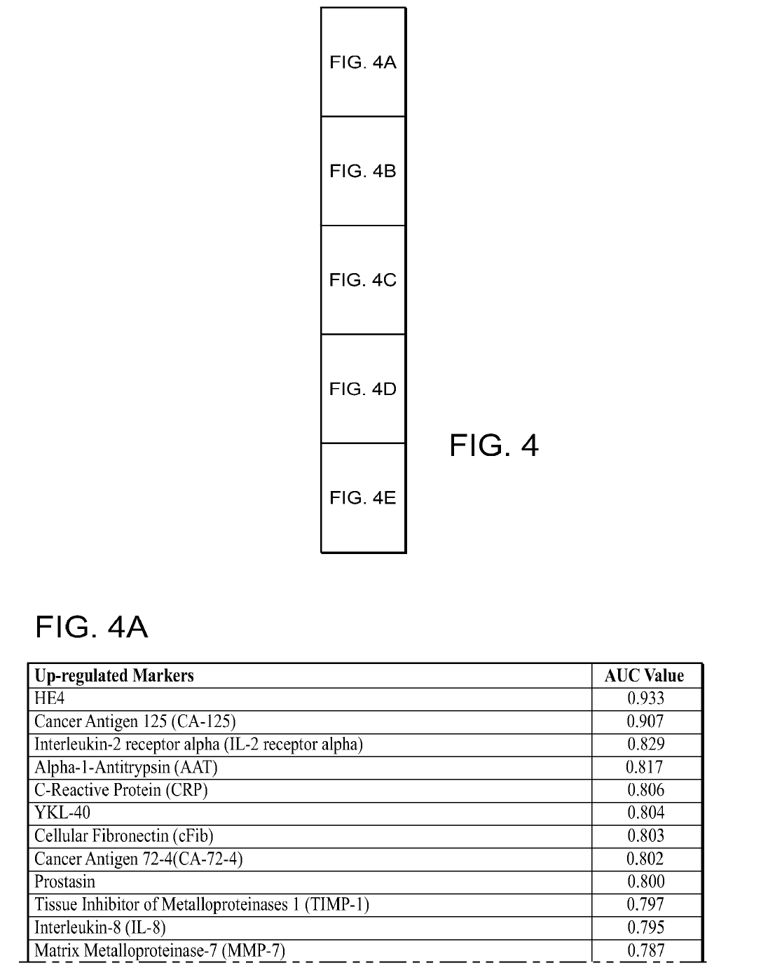

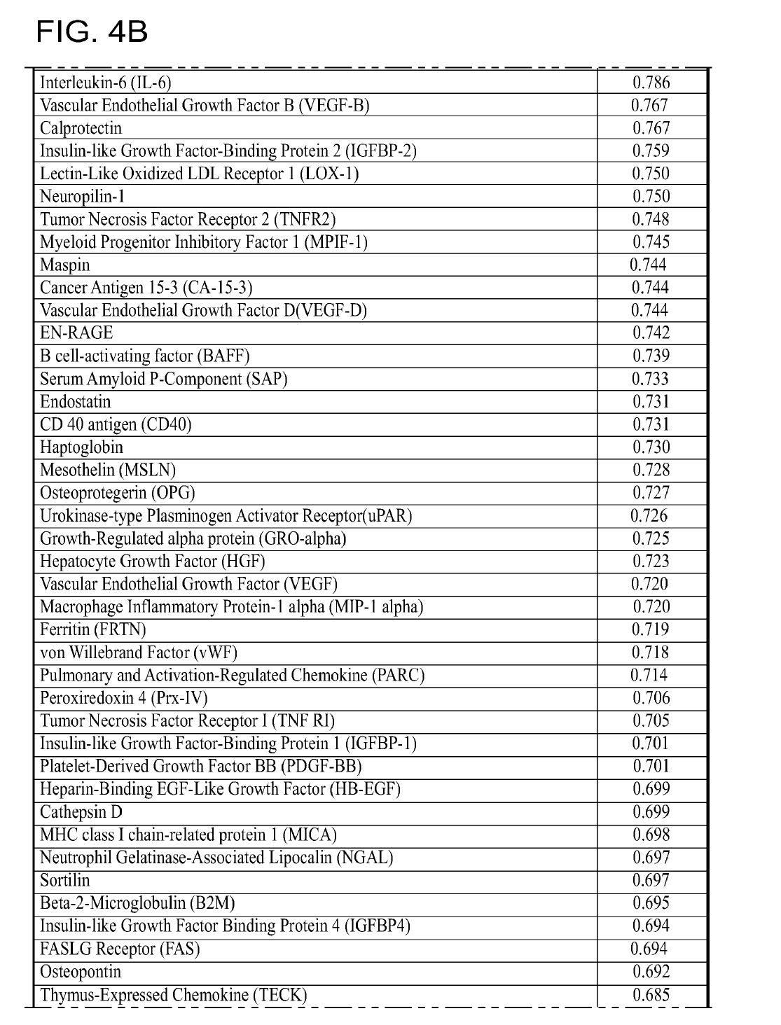

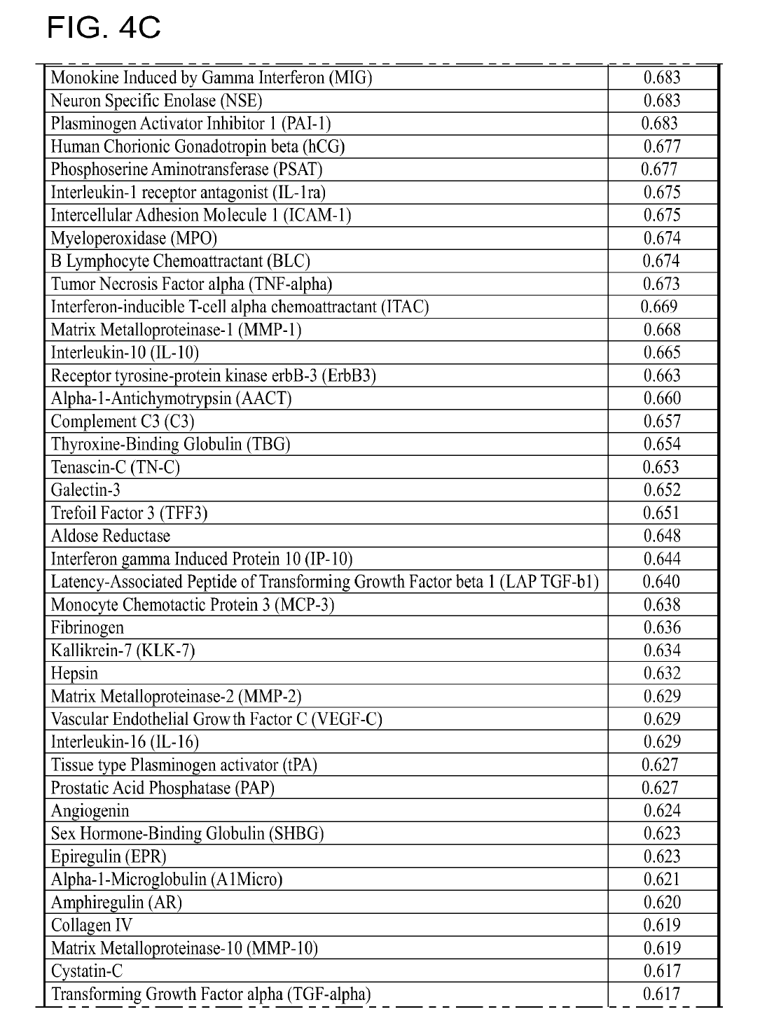

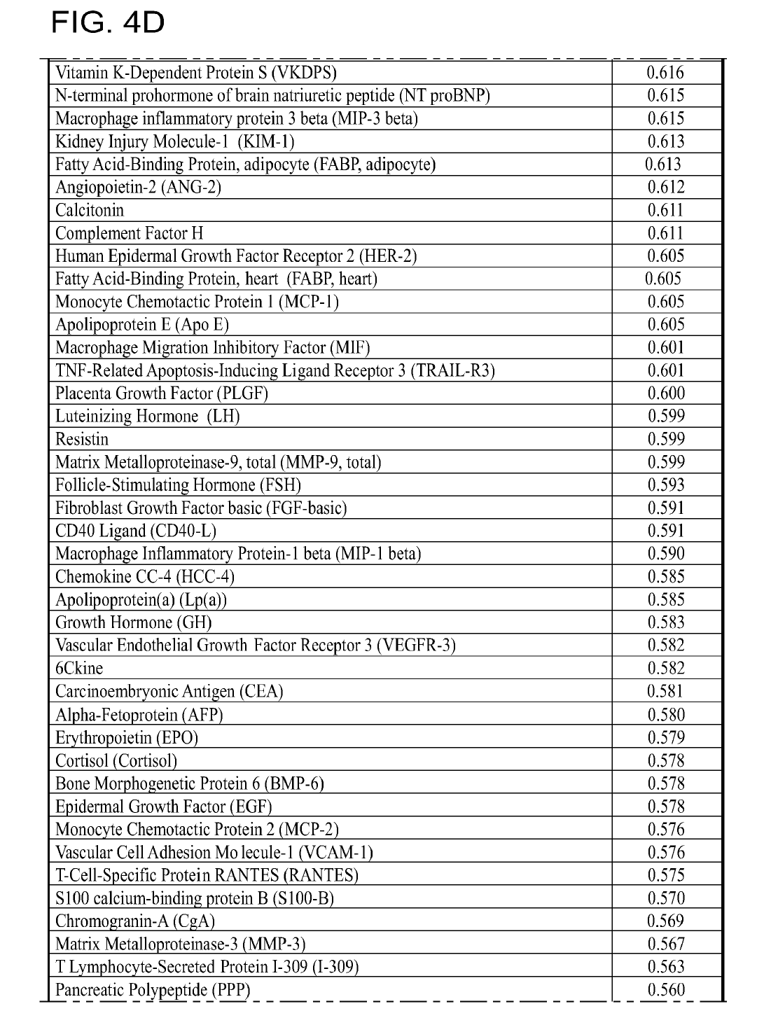

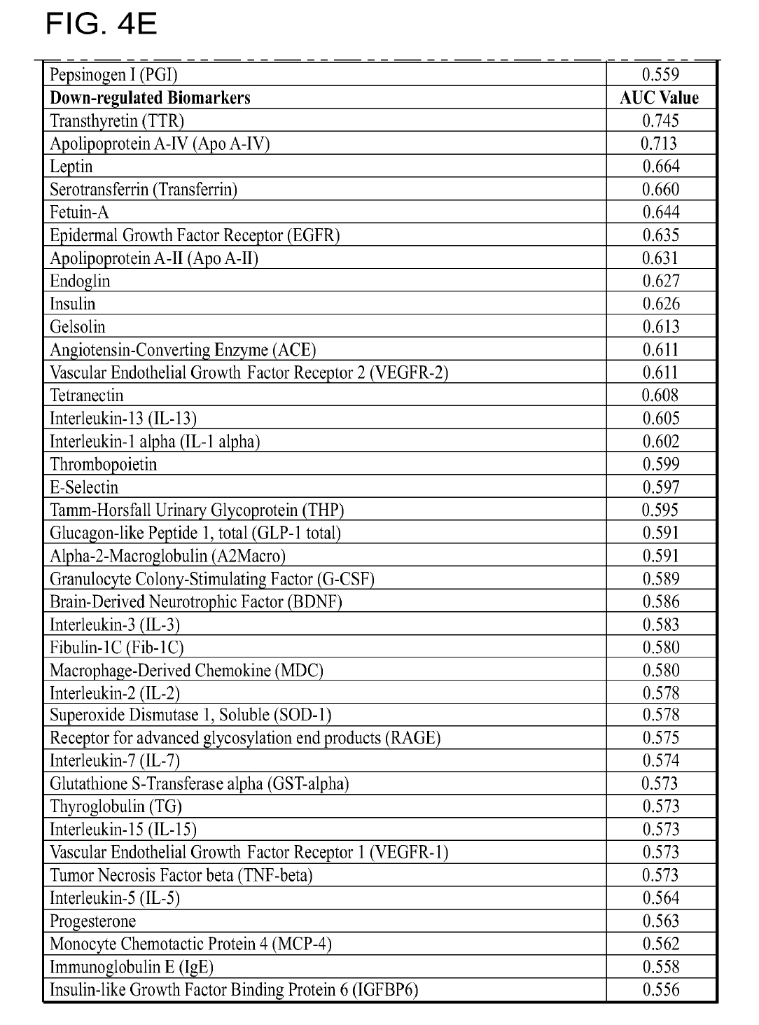

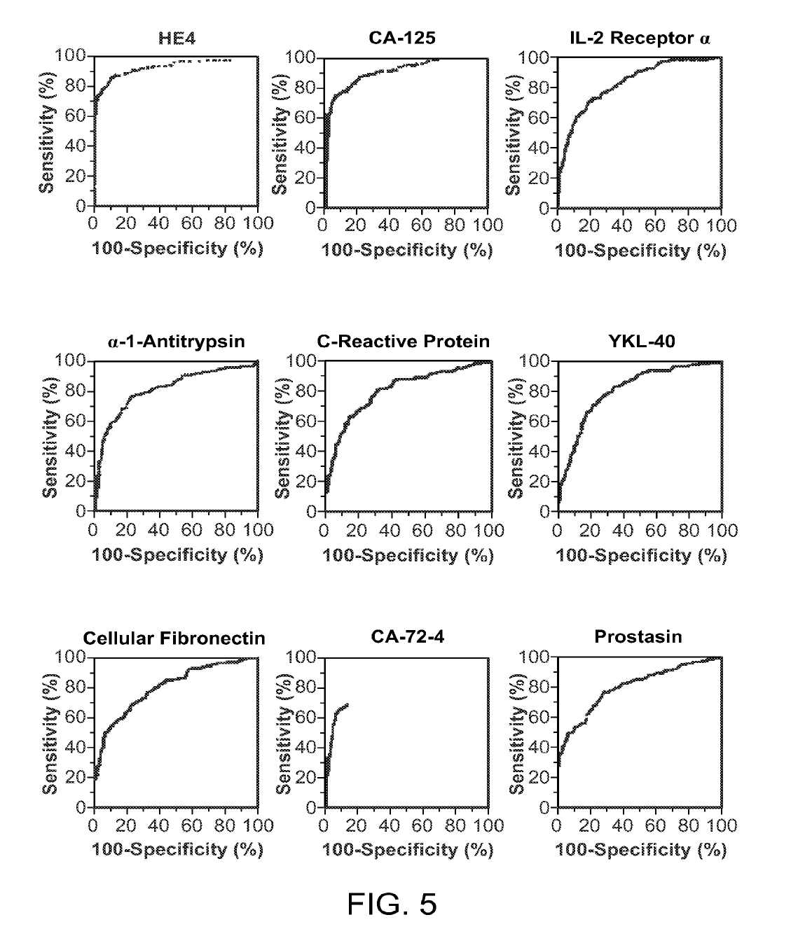

For each biomarker, an ROC curve was generated and its area under the curve (AUC) value compared to that of an uninformative marker (AUC=0.500). A total of 175 biomarkers were dysregulated (P-values>0.05) in the ovarian cancer samples relative to the benign gynecological conditions. Of these, 136 biomarkers were up-regulated and 39 down-regulated (FIGS. 3 and 4). The biomarkers with the greatest AUC values were predominantly up-regulated in ovarian cancer (FIGS. 3, 4, and 5) with values ranging from 0.599 to 0.933. The most up-regulated markers were HE4 and CA-125 with AUC values of 0.933 and 0.907, respectively, followed by interleukin-2 receptor α (IL-2 receptor α), α1-antitrypsin, C-reactive protein, YKL-40, cellular fibronectin, cancer antigen 72-4 (CA-72-4) and prostasin, with AUC values between 0.829 and 0.800 (FIG. 3). The remaining 127 up-regulated biomarkers had a continuum of AUC values from 0.797 to 0.556 (FIG. 4). Thirty-four of the remaining 127 markers had AUC values above 0.700. For down-regulated biomarkers, the AUC values ranged from 0.556 to 0.745 (FIG. 4). The two most informative of these stood out as transthyretin (0.745) and apolipoprotein A-IV (0.713), while the remaining biomarkers had AUC values below 0.700.

This is the first time that thirteen of the twenty biomarkers with the highest AUC values, namely HE4, IL-2 receptor α, YKL-40, cellular fibronectin, CA 72-4, prostasin, MMP-7, VEGF-B, Calprotectin, IGFBP-2, LOX-1, neuropilin-1 and MPIF-1 have been accurately quantified together, on a coherent set of samples, under uniformly controlled analytical conditions, to determine their discriminative power for ovarian cancer. This approach improves biomarker comparisons and should aid in the selection of biomarkers in the development of multi-biomarker panels.

As a comparison between the two most informative biomarkers in this study, the sensitivity for HE4 and CA-125 was determined over a range of specificity values. In addition, the optimal cut-off value, defined as that yielding the greatest sum of specificity and sensitivity was calculated for each biomarker. The sensitivity for HE4 alone decreased from 89.0% to 57.1% as specificity increased from 80% to 99.6%, while for CA-125 alone the sensitivity decreased from 85.2% to 30.2%. The optimal cut-off for HFA and CA-125 was 54.8 pM and 52.5 U/mL, respectively giving sensitivity values of 86.6% and 74.5%, respectively, and specificity values of 89.4% and 93.7%, respectively. As expected from ROC curves, there are trade-offs when no individual biomarker shows high specificity at a predetermined high sensitivity value. For example, at 100% sensitivity, both HE4 and CA-125 were 0% specific. At 98% sensitivity, HE4 had 30.6% specificity and CA-125 had 35.4% specificity. However, to see relatively good specificity values, the sensitivities had to be lowered to approximately 95%. At 95% sensitivity, HFA had 50.9% specificity and CA-125 had 45.4% specificity. These values, along with the AUC values, indicated that on this population, HE4 performed slightly better than CA-125. In addition, these results show that none of the biomarkers in this study are sufficiently informative as standalone ovarian cancer biomarkers for broad applications and that biomarker panels are needed to improve performance to clinically acceptable levels.

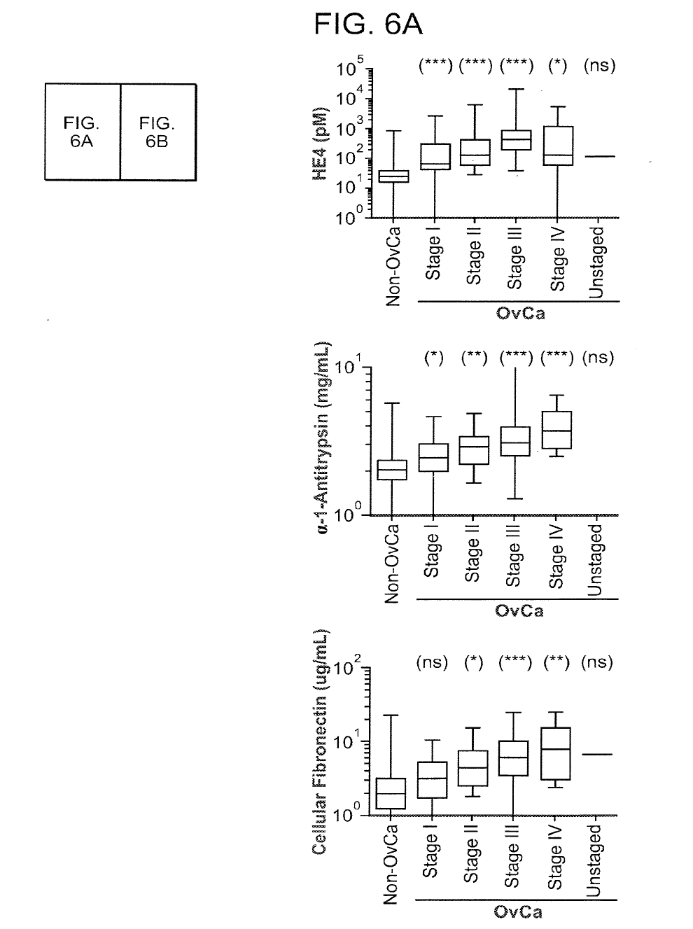

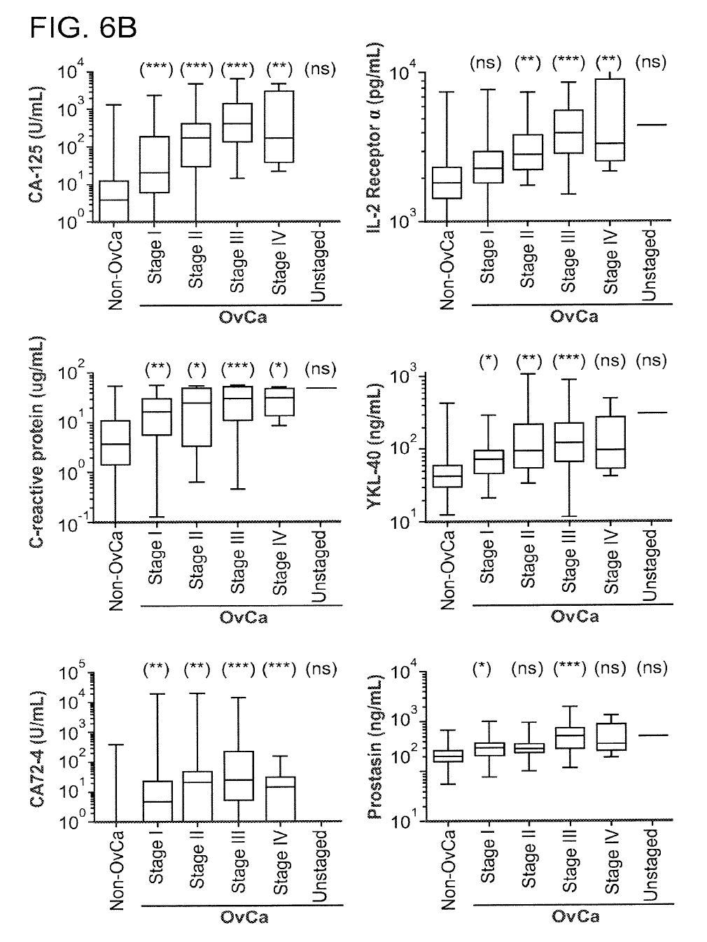

To determine if some biomarkers might have greater discrimination for different stages of cancer, especially early stage, the nine biomarkers with AUC values above 0.800 on FIGO stage I and II samples were compared where there is the greatest need for marker-based detection (FIG. 6). For FIGO stage I samples, both HE4 and CA-125 were highly discriminative (P-values<0.001), followed in descending order by C-reactive protein and CA 72-4 (P-values 0.001-0.01) then α1-antitrypsin, YKL-40 and prostasin (P-values 0.01-0.05). For IL2-receptor α and cellular fibronectin, there were no statistical differences between stage I cancer and benign conditions (P-values>0.05). For FIGO stage II samples, both HE4 and CA-125 were again highly discriminative (P-values<0.001), followed by for IL2-receptor α, α1-antitrypsin, YKL-40 and CA 72-4 (P-values 0.001-0.01) and then C-reactive protein and cellular fibronectin (P-values 0.01-0.05). For prostasin, there was no statistical difference (P-value>0.05).

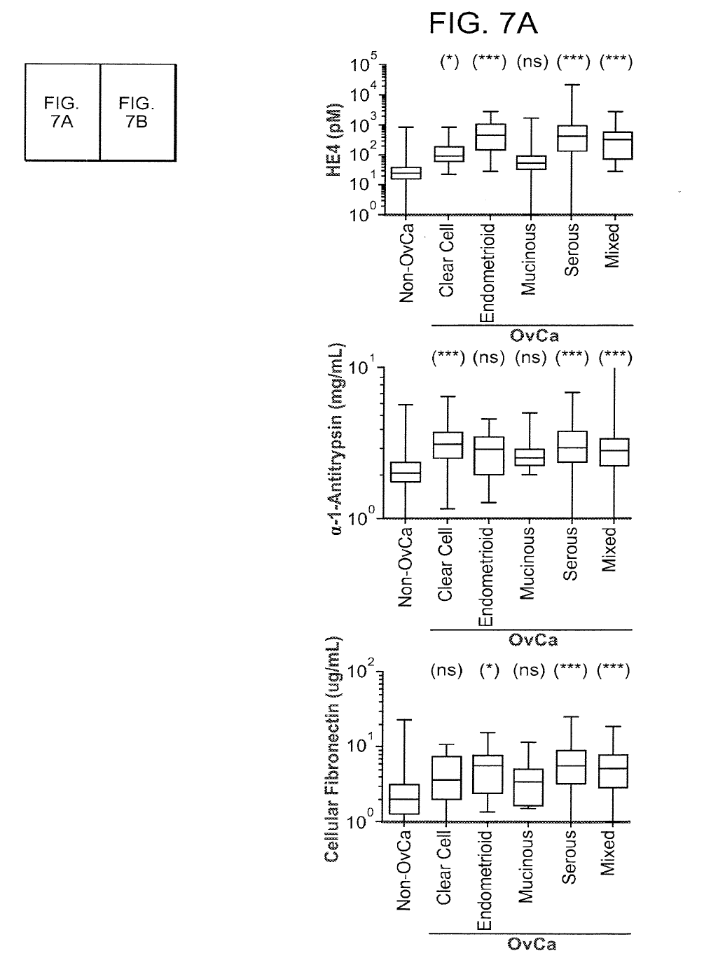

The same nine biomarkers were evaluated to determine if there were statistically significant differences between samples from women with benign conditions and women with each individual subtype of ovarian cancer (FIG. 7). For clear cell carcinomas, α1-antitrypsin and C-reactive protein were highly discriminatory (P-values<0.001), followed in descending order by HE4, CA-125 and IL2-receptor α (P-values 0.01-0.05). For YKL-40, cellular fibronectin, CA 72-4 and prostasin there were no statistical differences (P-value>0.05). For endometrioid carcinomas, there were highly significant differences for HE4 and CA-125 (P-values<0.001) and significant differences for C-reactive protein, cellular fibronectin, CA 72-4 (P-values 0.01-0.05). For α1-antitrypsin, IL2-receptor α, YKL-40 and prostasin there were no statistical differences (P-values>0.05). For mucinous carcinomas, only CA 72-4 had a significant difference (P-value 0.01-0.05). For serous and mixed carcinomas, all nine biomarkers had highly significant differences (P-value<0.001). Therefore, with the exception of mucinous carcinomas, the nine biomarkers are informative for all common ovarian cancer subtypes, however, their different discriminative powers suggests that different combinations of markers may be useful for different subtypes. While it would have been preferential to find more informative biomarkers for the mucinous subtype, it is relatively rare. Indeed, only 6.0% of the cancers in the study were of mucinous subtype (FIG. 1).

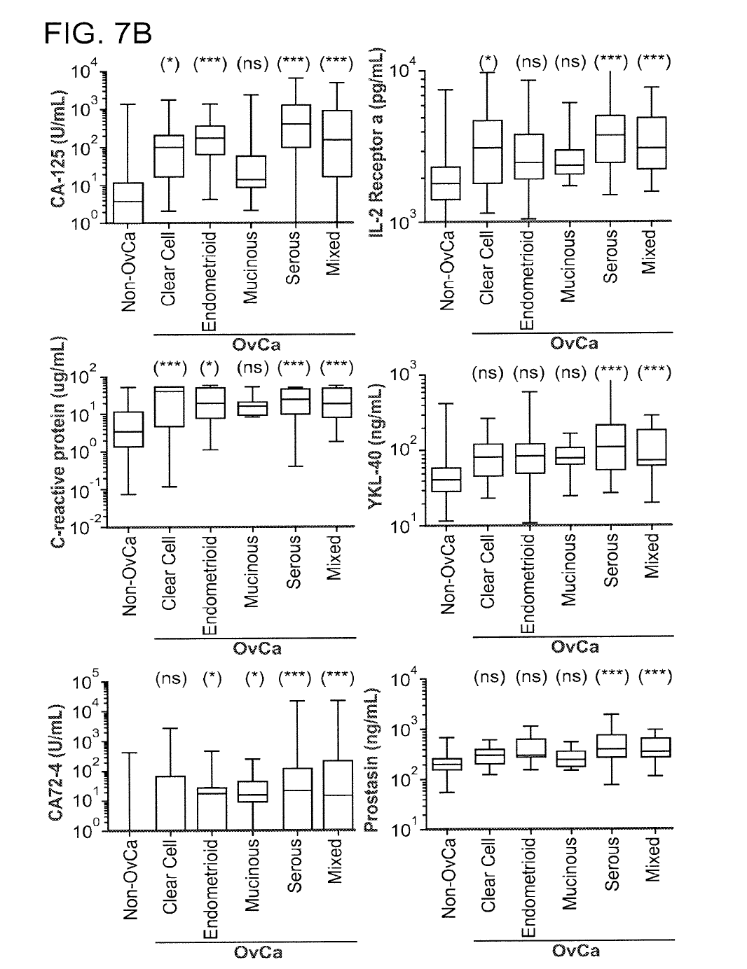

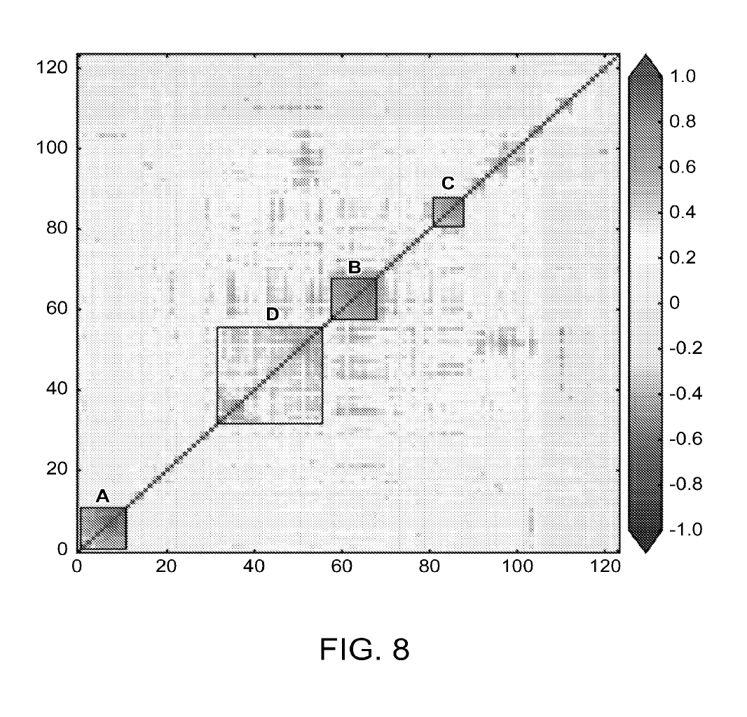

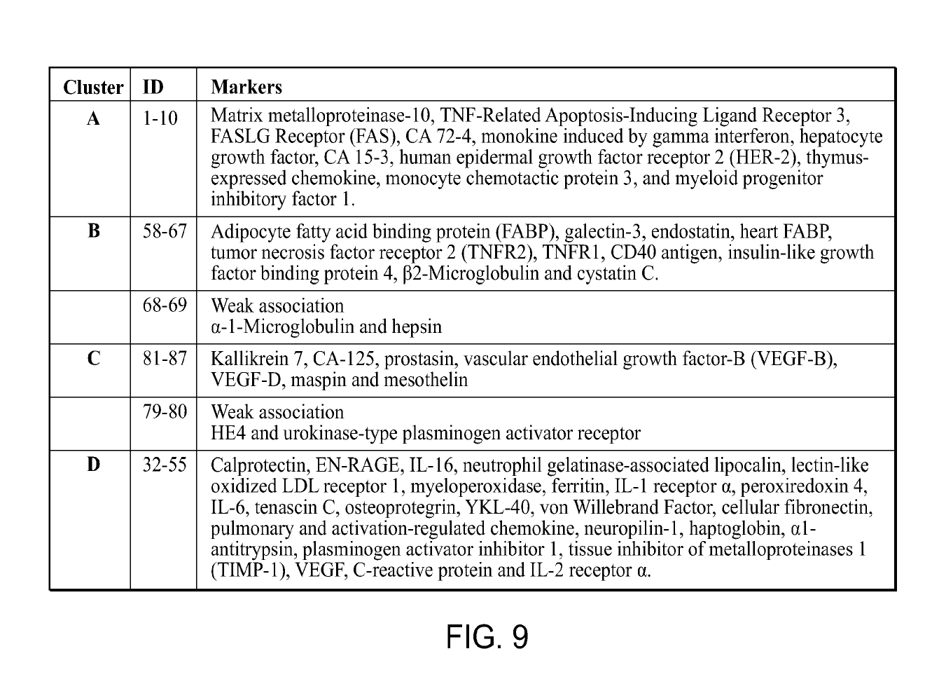

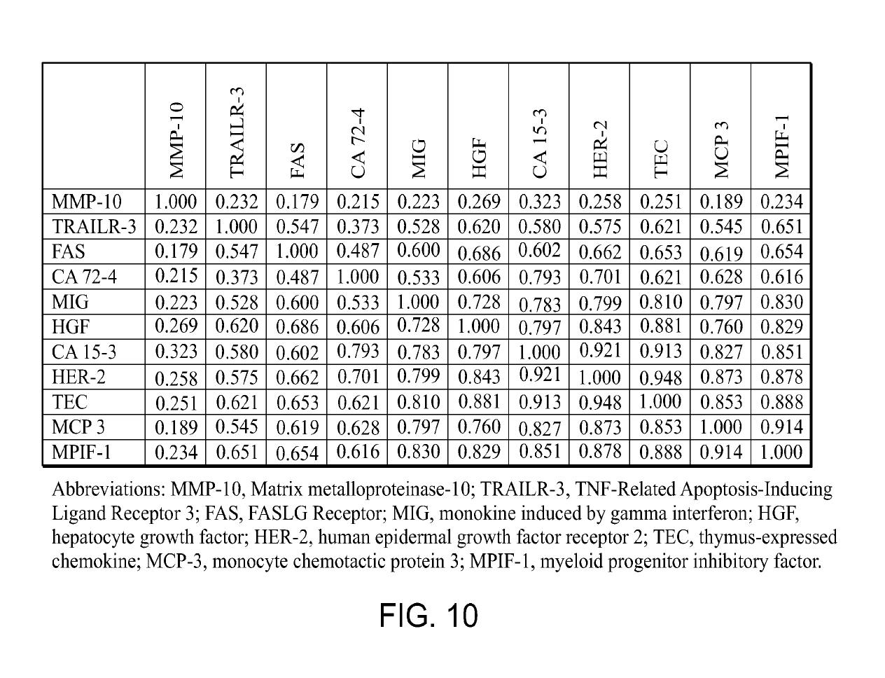

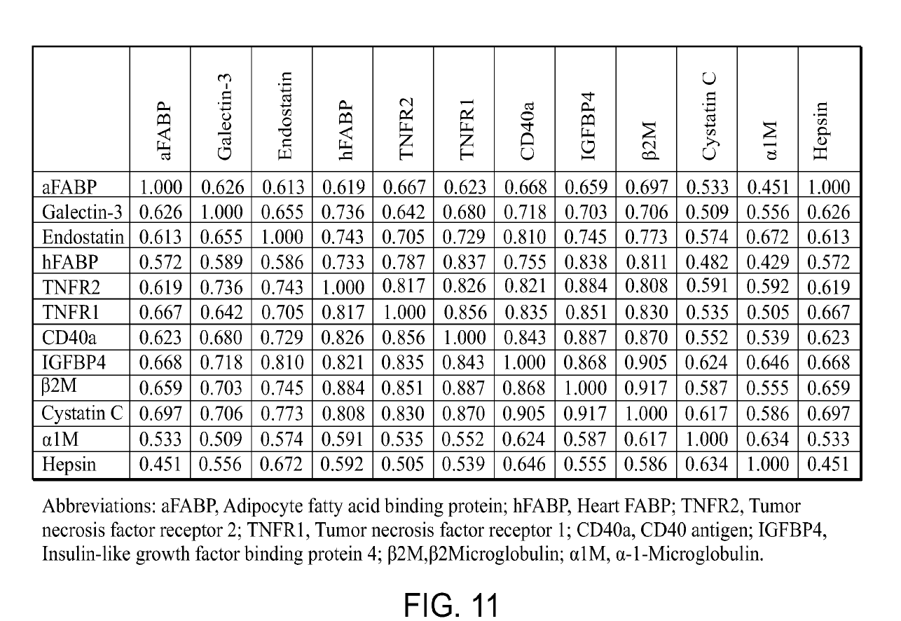

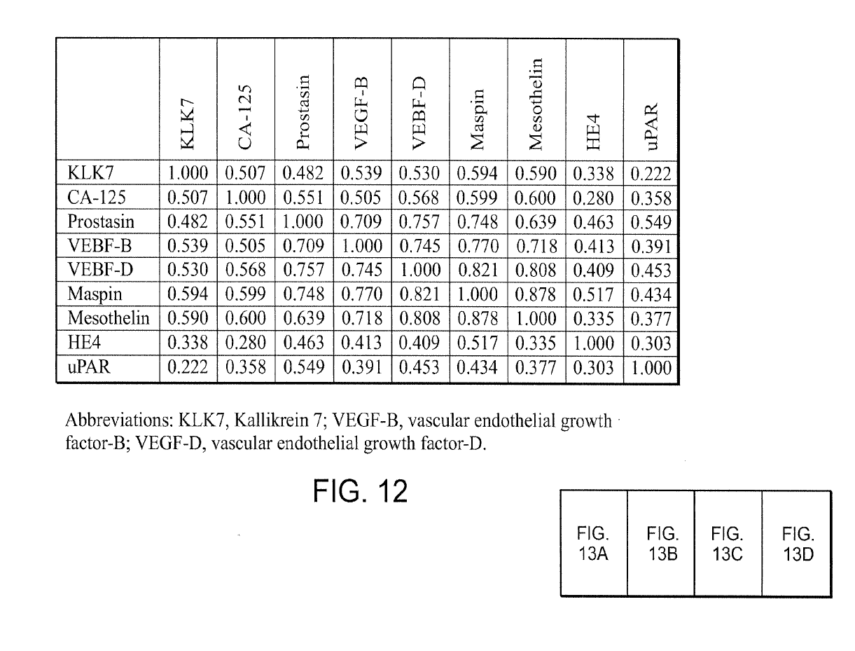

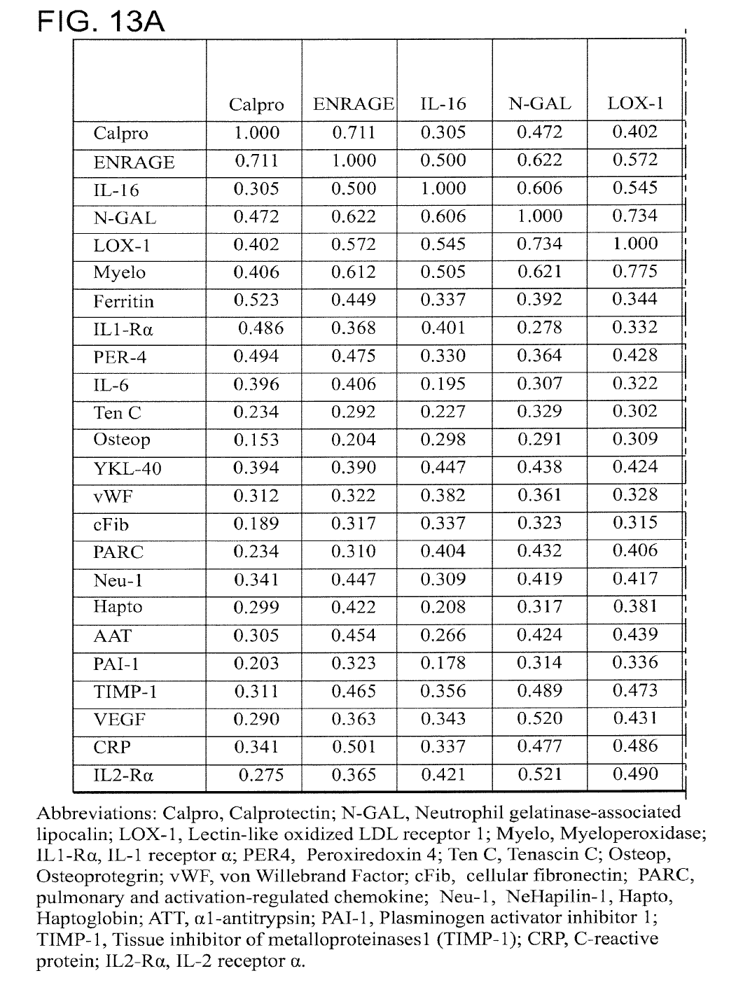

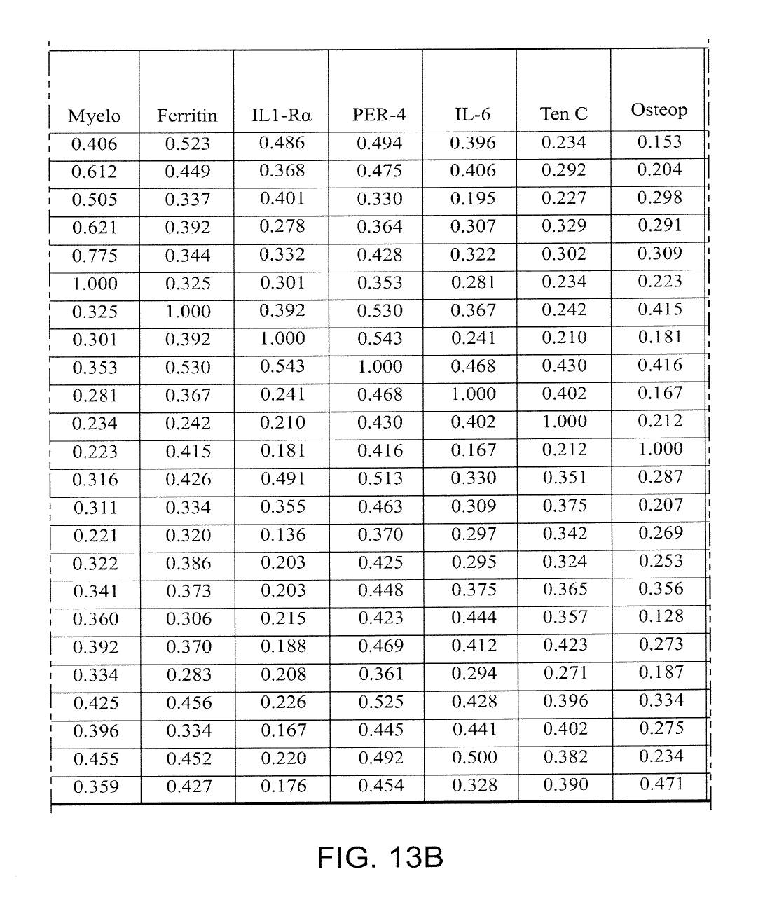

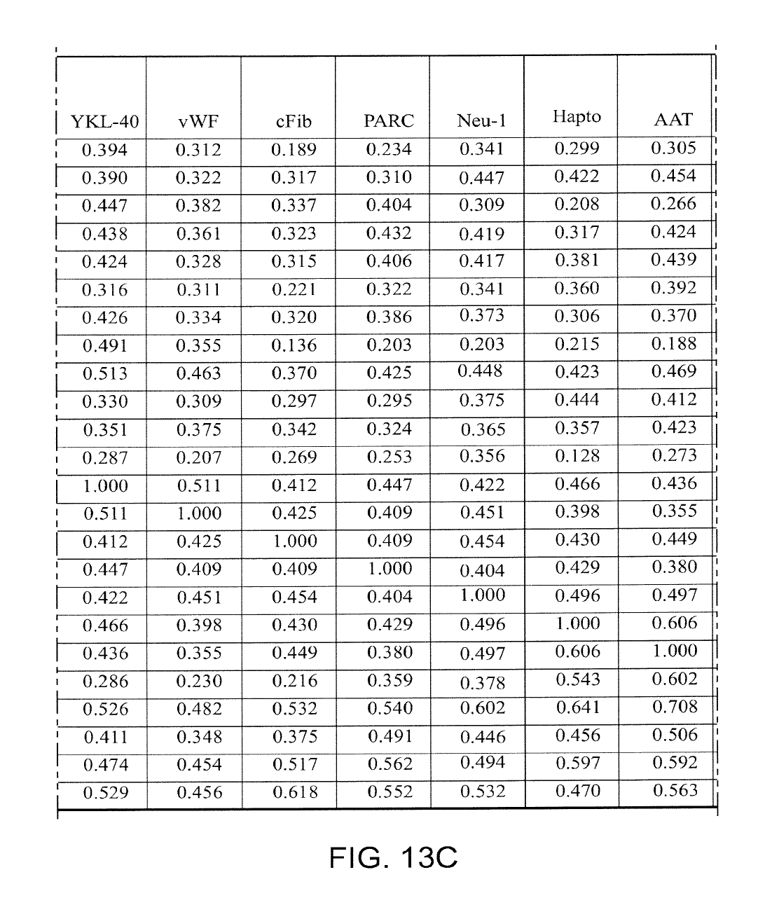

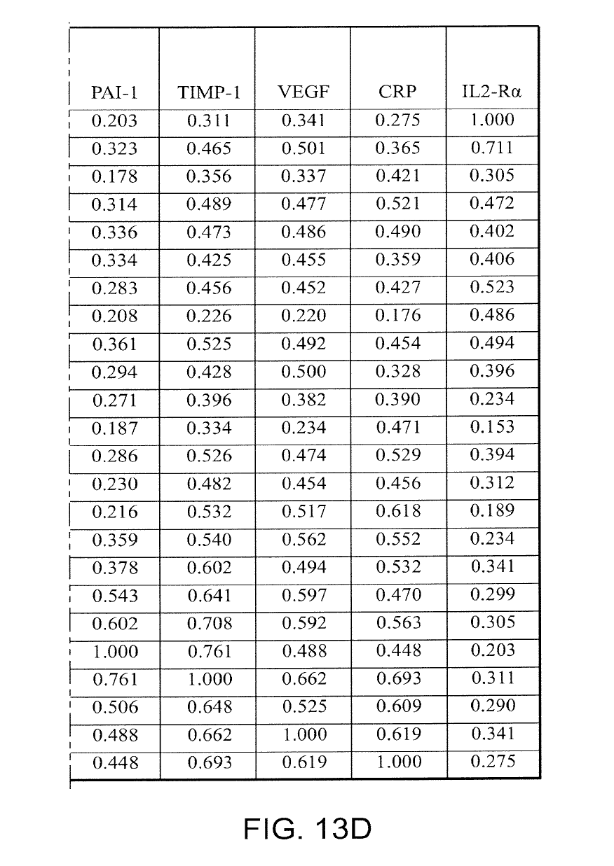

For simplicity and cost effectiveness, the use of a single biomarker is preferred over multiple biomarkers. However, it is clear that single biomarkers may not be able to capture the inherent diversity of complexes diseases such as ovarian cancer. An informative test seeks to combine multiple biomarkers in a way that each marker adds a different type of discrimination either to the entire patient population or the population subdivisions made by the other markers. Simply put, markers with poor correlation with one another have a greater chance of individually contributing to a panel than markers with strong correlation with one another. Therefore, correlation analysis was performed on the strongest ovarian cancer markers—the 124 biomarkers with AUC values greater than 0.600. The co-varying molecules were sorted agglomeratively with hierarchical clustering using Pearson correlation coefficients as the distance measure. The pair-wise results were assembled into a 124×124 matrix (numbered 0-123) and displayed using a heat map where an intense red color signifies strong positive correlation and blue signifies a negative correlation (FIG. 8). There were four major clusters (Clusters A through D; FIGS. 8-13), each cluster representing markers strongly correlating with each other. Each of these clusters contained markers that are strong ovarian cancer markers. Cluster A (markers 1-10) contained two strong ovarian cancer markers, CA 72-4 and MPIF-1 (FIGS. 9 and 10). TNFR2 was found in Cluster B (markers 58-67; Figures XXX (Tables S3 and S5). Cluster C (markers 79-87) contained the two strongest ovarian cancer markers (HFA, CA-125) as well as prostasin and VEGF-B (FIGS. 9 and 12). The strongest correlations with CA-125 were mesothelin (Pearson correlation coefficient=0.600), maspin (0.599), VEGF-D (0.568), prostasin (0.551), kallikrein-7 (0.507) and VEGF-B (0.505). Maspin (0.517) correlated with HE4 the strongest, followed by TIMP-1 (0.470), prostasin (0.463), IL-2 receptor α (0.424), VEGF-B (0.413) and VEGF-D (0.409). Finally, the largest cluster (Cluster D; biomarkers 32-55), was composed of loosely correlated markers that contained several good ovarian cancer markers including calprotectin, LOX-1, IL-6, YKL-40, cellular fibronectin, neuropilin-1, α1-antitrypsin, TIMP-1, C-reactive protein and IL-2 receptor α (FIGS. 9 and 13). These correlation data can help drive the development of biomarkers panels and may give insights into pathways that are disrupted in ovarian cancer.

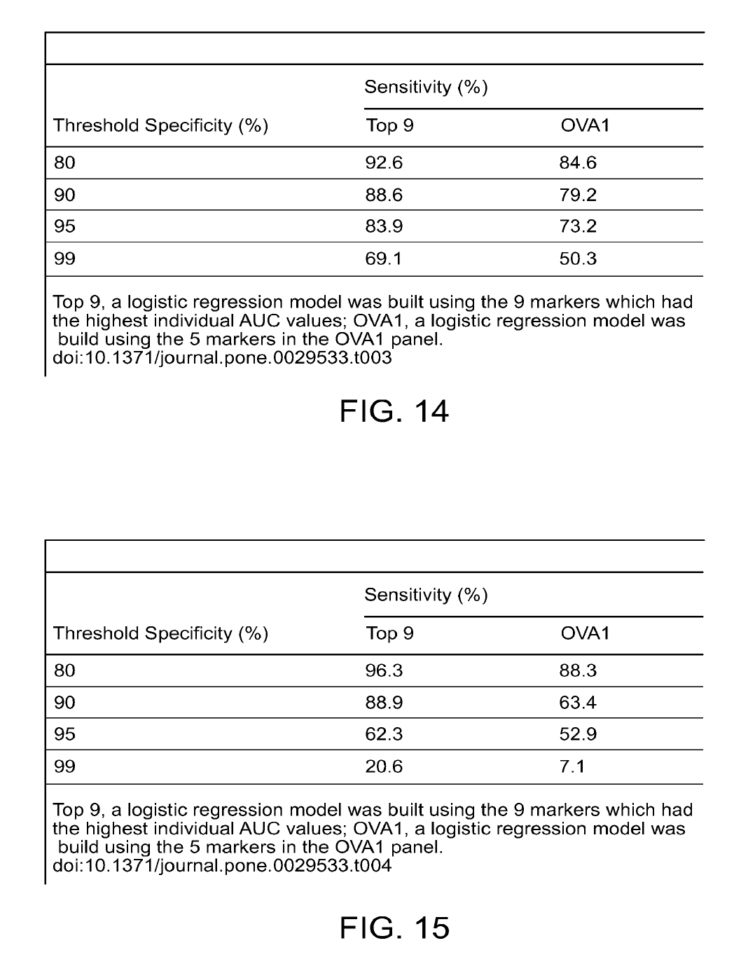

The combined performance of the nine markers with AUC values greater than 0.800 were evaluated to determine the predictive value of a simple multi-marker scenario. The nine markers were combined using logistic regression which yielded an AUC of 0.950 (Standard error: 0.01213; 95% CI: 0.926-0.974; P-value: <0.0001). Next this performance was compared against the five markers in the FDA-cleared OVA1 test. The samples in this study were collected by gynecologic oncologists. A similar study population was reported in the OVA1 510(k) summary with 100% sensitivity (invasive ovarian cancer only) and 32.9% specificity. The five markers combined and a logistic regression model was built. Consistent with the OVA1 510(k) summary, with this sample set, at 32.9% specificity, OVA1 biomarkers gave a sensitivity of 98.0%. Interestingly, with these samples, at a specificity of 32.9%, CA-125 alone had a sensitivity of 98.0%. This indicated that the additional OVA markers contributed little, if any, to the overall classification. Indeed, the AUC value for the five OVA1 biomarkers was 0.912 (Standard error 0.0157; 95% CI: 0.881-0.943; P-value: <0.0001), barely higher than CA-125 alone which had an AUC of 0.907 (Standard error 0.01571; 95% CI: 0.877-0.938; P-value: <0.0001). The two models were further compared by determining the sensitivity of models at fixed specificity values and the specificity of models at fixed sensitivity values (FIGS. 14 and 15). In general, the logistic regression model built on the top 9 markers outperformed the model built on OVA1 markers at all points of the ROC curve. At fixed specificity values between 80 and 95%, the top 9 model was 8 to 10% more sensitive that the model built on the OVA1 markers. At higher specificity (99%), the top 9 model was approximately 19% more sensitive. At fixed sensitivity between 80 and 99%, the top 9 model was between 8 and 25% more specific than the model built on the OVA1 markers.

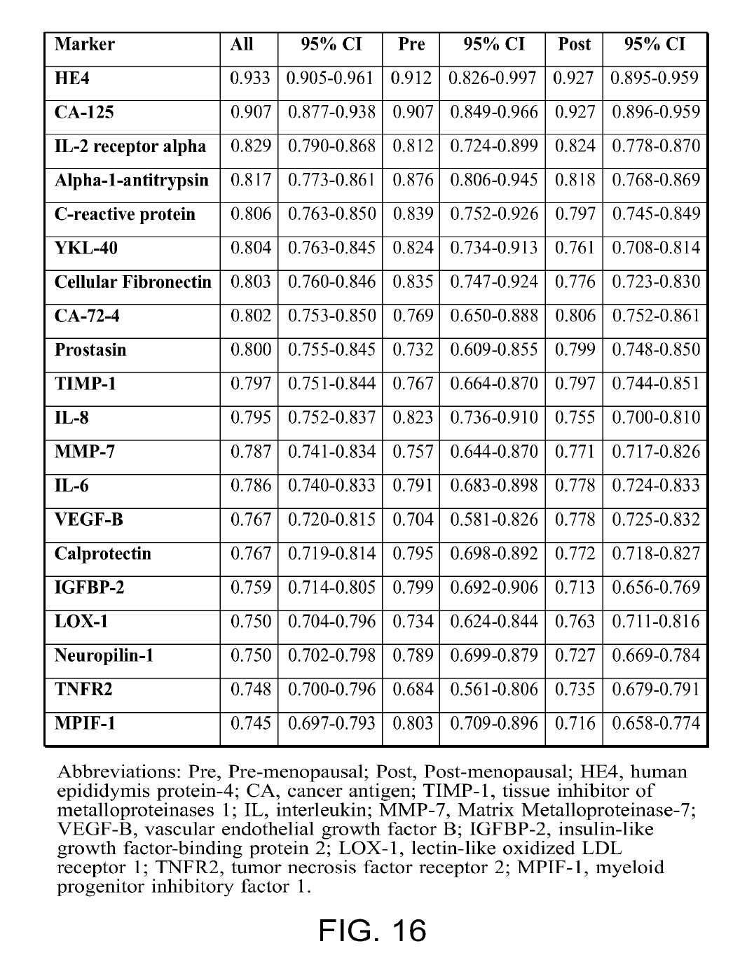

As both the top nine and OVA1 panels contained markers that may perform differently for pre- and post-menopausal women, the performance of the two panels were compared by menopausal status. For the top nine panel, the AUC value for pre-menopausal women was lower (0.937) than for post-menopausal women (0.953). This is consistent with the individual marker analysis that demonstrated that the top three individual markers (HE4, CA-125 and IL2-Rα) all performed better for the post-menopausal women (0.927, 0.927 and 0.824, respectively; (FIG. 16) than for the pre-menopausal women (0.912, 0.907 and 0.812, respectively). For the OVA1 panel, the AUC value for pre-menopausal women was slightly lower (0.920) than for post-menopausal women (0.924). Again, this is consistent with the individual marker analysis that demonstrated that CA-125, the marker that appears to drive the performance of the OVA 1 panel, performed worse for the group of pre-menopausal women (0.907) than for post-menopausal women (0.927).

New biomarkers have been identified that are capable of discriminating between samples drawn from women with benign ovarian conditions and those from women with ovarian cancer. Preliminary multivariate analysis, using a logistic regression model on the nine most informative biomarkers appeared to have significantly improved performance over the OVA1 biomarkers. This analysis indicates that our data have the potential to improve on OVA1 and other tests.

1. A method of determining the ovarian cancer status of a subject, comprising the steps of:
measuring the level of CA-125, HE4 and one or more biomarkers selected from the group consisting of IL-2 receptor alpha (IL-2Ra), Alpha-1-Antitrypsin (AAT), C-Reactive Protein (CRP), YKL-40, Cellular Fibronectin (cFib), prostasin, Tissue Inhibitor of Metalloproteinases 1 (TIMP-1), IL-8, IL-6, Vascular Endothelial Growth Factor B (VEGF-B), Matrix Metalloproteinase-7 (MMP-7), calprotectin, Insulin-like Growth Factor-Binding Protein 2 (IGFBP-2), Lectin-Like Oxidized LDL Receptor 1 (LOX-1), neuropilin-1, TNFR2, and MPIF-1 in a biological sample obtained from the subject; and
correlating the measurements with ovarian cancer status.

2. The method of claim 1, further comprising measuring the level of Cancer Antigen 72-4 (CA-72-4).

3. The method of claim 1, wherein the ovarian cancer status is the presence, absence, or risk of ovarian cancer.

4. The method of claim 1 further comprising:
managing subject treatment based on the status.

5. The method of claim 4, wherein managing subject treatment is selected from the group consisting of ordering more tests, performing surgery, and taking no further action.

6. The method of claim 4 further comprising:
measuring the level of said biomarkers after subject management;
correlating the measurements with ovarian cancer status; and
determining if subject management resulted in a change in ovarian cancer status.

7. The method of claim 1, wherein measuring is selected from detecting the presence or absence of the biomarkers, quantifying the amount of biomarkers, and qualifying the type of biomarker.

8. The method of claim 1, wherein the biomarkers are measured by an immunoassay and mass spectroscopy.

9. The method of claim 1, wherein the correlating is performed by a software classification algorithm.

10. The method of claim 1, wherein the sample is selected from blood, serum, and plasma.

11. A kit comprising:
a) a panel of affinity reagents that each selectively binds to CA-125, HE4 and one or more biomarkers selected from the group consisting of Interleukin-2 receptor alpha (IL-2 receptor alpha), Alpha-1-Antitrypsin (A AT), C-Reactive Protein (CRP), YKL-40, Cellular Fibronectin (cFib), Cancer Antigen 72-4 (CA-72-4), prostasin, Tissue Inhibitor of Metalloproteinases 1 (TIMP-1), IL-8, Matrix Metalloproteinase-7 (MMP-7), IL-6, Vascular Endothelial Growth Factor B (VEGF-B), calprotectin, Insulin-like Growth Factor-Binding Protein 2 (IGFBP-2), Lectin-Like Oxidized LDL Receptor 1 (LOX-1), neuropilin-1, TNFR2, and MPIF-1; and
b) instructions for the use of said panel in the method of claim 1.

12. The kit of claim 11, wherein the affinity reagent is an antibody.

13-14. (canceled)

15. A panel of biomarkers comprising CA-125, HE4, and one or more biomarkers selected from the group consisting of Interleukin-2 receptor alpha (IL-2 receptor alpha), Alpha-1-Antitrypsin (A AT), C-Reactive Protein (CRP), YKL-40, Cellular Fibronectin (cFib), Cancer Antigen 72-4 (CA-72-4), prostasin, Tissue Inhibitor of Metalloproteinases 1 (TIMP-1), IL-8, Matrix Metalloproteinase-7 (MMP-7), IL-6, Vascular Endothelial Growth Factor B (VEGF-B), calprotectin, Insulin-like Growth Factor-Binding Protein 2 (IGFBP-2), Lectin-Like Oxidized LDL Receptor 1 (LOX-1), neuropilin-1, TNFR2, and MPIF-1.

16. (canceled)

17. The method of claim 1, wherein the subject is a postmenopausal subject, and the biomarkers measured are CA-125, HE4 and IL2-Ralpha.

18. The method of claim 1, wherein the subject has early stage ovarian cancer, and the biomarkers measured are HE4, CA 125, and CRP or CA-72-4.
